(19) United States
(12) Patent Application Publication
(10) Pub. No.: US 2015/0337329 A1
(43) Pub. Date: Nov. 26, 2015

(54) GENE SEQUENCES AND USES THEREOF IN PLANTS

(71) Applicant: Monsanto Technology LLC, St. Louis, MO (US)

(72) Inventor: Michael D. Edgerton, St. Louis, MO (US)

(21) Appl. No.: 14/545,275

(22) Filed: Apr. 15, 2015

Related U.S. Application Data
(62) Division of application No. 13/470,467, filed on May 14, 2012.
Application No. 13/470,467 is a continuation of application No. 13/046,601, filed on Mar. 11, 2011, now abandoned.
Application No. 13/046,601 is a continuation of application No. 11/879,785, filed on Jul. 18, 2007, now abandoned.
Application No. 11/879,785 is a continuation-in-part of application No. 10/310,154, filed on Dec. 4, 2002, now abandoned.
(60) Provisional application No. 60/337,358, filed on Dec. 4, 2001.

(51) Int. Cl. C12N 15/82; C12N 9/02

(52) U.S. Cl. CPC C12N 15/8279; C12N 15/8274; C12N 15/8275; C12N 15/8261; C12N 15/8273; C12N 15/8241; C12N 9/0071; C12Y 114/12017

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced traits.

### CROSS-REFERENCE TO RELATED APPLICATIONS

This application is a divisional of U.S. application Ser. No. 13/470,467, filed May 14, 2012, which application filed is a continuation of U.S. application Ser. No. 13/046,601, filed Mar. 11, 2011 (abandoned), which application is a continuation of U.S. application Ser. No. 11/879,785, filed Jul. 18, 2007 (abandoned), which application is a continuation-in-part of U.S. application Ser. No. 10/310,154 filed Dec. 4, 2002 (abandoned), which application claims the benefit of priority of U.S. Provisional Application No. 60/337,358, filed Dec. 4, 2001, all of which applications are incorporated herein by reference in their entirety.

### INCORPORATION OF SEQUENCE LISTING

Two copies of the sequence listing (COPY 1 and COPY 2) and a computer readable form (CRF) of the sequence listing, all on CD-Rs, each containing the text file named 1436916.TXT, which is 87,273,472 bytes (measured in MS-WINDOWS), were created on Apr. 14, 2015 and are herein incorporated by reference.

### INCORPORATION OF COMPUTER PROGRAM LISTING

Two copies of the Computer Program Listing (Copy 1 and Copy 2) containing folders hmmer-2.3.2 and 164pfamDir, all on CD-Rs were filed on Jul. 18, 2007 with the application accorded Ser. No. 11/879,785 (abandoned), which were incorporated by reference into the application accorded Ser. No. 13/046,601 (filed Mar. 1, 2011; abandoned), which were incorporated by reference into the application accorded Ser. No. 13/470,467 (filed May 14, 2012) and which are incorporated herein by reference in their entirety. Folder hmmer-2.3.2 contains the source code and other associated file for implementing the HMMer software for Pfam analysis. Folder 164pfamDir contains 164 Pfam Hidden Markov Models. Both folders were created on CD-R on Jul. 17, 2007, having a total size of Ser. No. 15/204,353 bytes (measured in MS-WINDOWS).

LENGTHY TABLES
The patent application contains a lengthy table section. A copy of the table is available in electronic form from the USPTO web site (http://seqdata.uspto.gov/?pageRequest=docDetail&DocID=US20150337329A1). An electronic copy of the table will also be available from the USPTO upon request and payment of the fee set forth in 37 CFR 1.19(b)(3).

### INCORPORATION OF TABLES

Two copies of Table 7 (Copy 1 and Copy 2) and a computer readable form (CRF), all on CD-Rs, each containing the file named 1440025.txt, which is 294,912 bytes (measured in MS-WINDOWS), were created on Apr. 14, 2015, and comprise 68 pages when viewed in MS Word, are herein incorporated by reference.

### FIELD OF THE INVENTION

Disclosed herein are inventions in the field of plant genetics and developmental biology. More specifically, the present inventions provide plant cells with recombinant DNA for providing an enhanced trait in a transgenic plant, plants comprising such cells, seed and pollen derived from such plants, methods of making and using such cells, plants, seeds and pollen.

### BACKGROUND OF THE INVENTION

Transgenic plants with enhanced agronomic traits such as yield, environmental stress tolerance, pest resistance, herbicide tolerance, improved seed compositions, and the like are desired by both farmers and consumers. Although considerable efforts in plant breeding have provided significant gains in desired traits, the ability to introduce specific DNA into plant genomes provides further opportunities for generation of plants with improved and/or unique traits. The ability to develop transgenic plants with enhanced traits depends in part on the identification of useful recombinant DNA for production of transformed plants with enhanced properties, e.g. by actually selecting a transgenic plant from a screen for such enhanced property. An object of this invention is to provide transgenic plant cell nuclei, plant cells, plants and seeds by screening transgenic crop plants for one of more enhanced agronomic traits where the nucleus in cells of the plant or seed has recombinant DNA provided herein. A further object of the invention is to provide screening methods requiring routine experimentation by which such transgenic plant cell nuclei, cells, plants and seeds can be identified by making a reasonable number of transgenic events and engaging in screening identified in this specification and illustrated in the examples.

### SUMMARY OF THE INVENTION

This invention provides plant cell nuclei with recombinant DNA that imparts enhanced agronomic traits in transgenic plants having the nuclei in their cells, e.g. enhanced water use efficiency, enhanced cold tolerance, increased yield, enhanced nitrogen use efficiency, enhanced seed protein or enhanced seed oil. Such recombinant DNA in a plant cell nucleus of this invention is provided in as a construct comprising a promoter that is functional in plant cells and that is operably linked to DNA that encodes a protein. Such DNA in the construct is sometimes defined by protein domains of an encoded protein targeted for production or suppression, e.g. a “Pfam domain module” (as defined herein below) from the group of Pfam domain modules identified in Table 21 (page 72). Alternatively, e.g. where a Pfam domain module is not available, such DNA in the construct is defined a consensus amino acid sequence of an encoded protein that is targeted for production e.g. a protein having amino acid sequence with at least 90% identity to a consensus amino acid sequence in the group of SEQ ID NO: 24153 through SEQ ID NO: 24174. Alternatively, in other cases where neither a Pfam domain module nor a consensus amino acid sequence is available, such DNA in the construct is defined by the sequence of a specific encoded and/or its homologous proteins.

Other aspects of the invention are specifically directed to transgenic plant cells comprising the recombinant DNA of the invention, transgenic plants comprising a plurality of such plant cells, progeny transgenic seed, embryo and transgenic pollen from such plants. Such plant cells are selected from a population of transgenic plants regenerated from plant cells transformed with recombinant DNA and that express the protein by screening transgenic plants in the population for an enhanced trait as compared to control plants that do not have said recombinant DNA, where the enhanced trait is selected from group of enhanced traits consisting of enhanced water use efficiency, enhanced cold tolerance, increased yield, enhanced nitrogen use efficiency, enhanced seed protein and enhanced seed oil.

In yet another aspect of the invention the plant cells, plants, seeds, embryo and pollen further comprise DNA expressing a protein that provides tolerance from exposure to an herbicide applied at levels that are lethal to a wild type of said plant cell. Such tolerance is especially useful not only as an advantageous trait in such plants but is also useful in a selection step in the methods of the invention. In aspects of the invention the agent of such herbicide is a glyphosate, dicamba, or glufosinate compound.

Yet other aspects of the invention provide transgenic plants which are homozygous for the recombinant DNA and transgenic seed of the invention from corn, soybean, cotton, canola, alfalfa, wheat or rice plants.

This invention also provides methods for manufacturing non-natural, transgenic seed that can be used to produce a crop of transgenic plants with an enhanced trait resulting from expression of stably-integrated, recombinant DNA in the nucleus of the plant cells. More specifically the method comprises (a) screening a population of plants for an enhanced trait and recombinant DNA, where individual plants in the population can exhibit the trait at a level less than, essentially the same as or greater than the level that the trait is exhibited in control plants which do not express the recombinant DNA; (b) selecting from the population one or more plants that exhibit the trait at a level greater than the level that said trait is exhibited in control plants and (c) collecting seed from a selected plant. Such method further comprises steps (d) verifying that the recombinant DNA is stably integrated in said selected plants; and (e) analyzing tissue of a selected plant to determine the production of a protein having the function of a protein encoded by a recombinant DNA with a sequence of one of SEQ ID NO: 1-339; In one aspect of the invention the plants in the population further comprise DNA expressing a protein that provides tolerance to exposure to an herbicide applied at levels that are lethal to wild type plant cells and where the selecting is effected by treating the population with the herbicide, e.g. a glyphosate, dicamba, or glufosinate compound. In another aspect of the invention the transgenic plants are selected by identifying plants with the enhanced trait. The methods are especially useful for manufacturing corn, soybean, cotton, alfalfa, wheat or rice seed selected as having one of the enhanced traits described above.

Another aspect of the invention provides a method of producing hybrid corn seed comprising acquiring hybrid corn seed from a herbicide tolerant corn plant which also has stably-integrated, recombinant DNA comprising a promoter that is (a) functional in plant cells and (b) is operably linked to DNA that encodes a protein having at least one domain of amino acids in a sequence that exceeds the Pfam gathering cutoff for amino acid sequence alignment with a protein domain family identified by a Pfam name in the group of Pfam names identified in Table 12. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

### BRIEF DESCRIPTION OF THE DRAWINGS

FIG. 1 is a consensus amino acid sequence of SEQ ID NO: 358 and its homologs.

FIGS. 2-4 are plasmid maps.

### DETAILED DESCRIPTION OF THE INVENTION

In the attached sequence listing:

SEQ ID NO: 1-339 are nucleotide sequences of the coding strand of DNA for “genes” used in the recombinant DNA imparting an enhanced trait in plant cells, i.e. each represents a coding sequence for a protein;

SEQ ID NO: 340-678 are amino acid sequences of the cognate protein of the “genes” with nucleotide coding sequences 1-339;

SEQ ID NO: 679-24149 are amino acid sequences of homologous proteins;

SEQ ID NO: 24150 is a nucleotide sequence of a plasmid base vector useful for corn transformation;

SEQ ID NO: 24151 is a nucleotide sequence of a plasmid base vector useful for soybean transformation;

SEQ ID NO: 24152 is a nucleotide sequence of a plasmid base vector useful for cotton transformation; and

SEQ ID NO: 24153-24174 are consensus sequences.

Table 1 lists the protein SEQ ID Nos and their corresponding consensus SEQ ID Nos.

TABLE 1

| PEP SEQ | | Consensus |
|---|---|---|
| ID NO | Gene ID | SEQ ID NO |
| 357 | PHE0000025 | 24153 |
| 358 | PHE0000026 | 24154 |
| 369 | PHE0000033 | 24155 |
| 397 | PHE0000063 | 24156 |
| 468 | PHE0000168 | 24157 |
| 497 | PHE0000223 | 24158 |
| 508 | PHE0000235 | 24159 |
| 512 | PHE0000240 | 24160 |
| 514 | PHE0000242 | 24161 |
| 516 | PHE0000249 | 24162 |
| 518 | PHE0000251 | 24163 |
| 541 | PHE0000276 | 24164 |
| 551 | PHE0000289 | 24165 |
| 570 | PHE0000309 | 24166 |
| 578 | PHE0000317 | 24167 |
| 608 | PHE0000353 | 24168 |
| 645 | PHE0000421 | 24169 |
| 653 | PHE0000430 | 24170 |
| 658 | PHE0000435 | 24171 |
| 660 | PHE0000437 | 24172 |
| 668 | PHE0000454 | 24173 |
| 669 | PHE0000455 | 24174 |

### DETAILED DESCRIPTION OF THE INVENTION

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein “homolog” means a protein in a group of proteins that perform the same biological function, e.g. proteins that belong to the same Pfam protein family and that provide a common enhanced trait in transgenic plants of this invention. Homologs are expressed by homologous genes. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. Hence, a recombinant DNA molecule useful in the present invention may have any base sequence that has been changed from SEQ ID NO: 1 through SEQ ID NO: 339 substitution in accordance with degeneracy of the genetic code. Homologs are proteins that, when optimally aligned, have at least 60% identity, more preferably about 70% or higher, more preferably at least 80% and even more preferably at least 90% identity over the full length of a protein identified as being associated with imparting an enhanced trait when expressed in plant cells. Homologs include proteins with an amino acid sequence that has at least 90% identity to a consensus amino acid sequence of proteins and homologs disclosed herein.

Homologs are identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails search of the significant hits against a database of amino acid sequences from the base organism that are similar to the sequence of the query protein. A hit is a likely ortholog, when the reciprocal query's best hit is the query protein itself or a protein encoded by a duplicated gene after speciation. A further aspect of the invention comprises functional homolog proteins that differ in one or more amino acids from those of disclosed protein as the result of conservative amino acid substitutions, for example substitutions are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants of the invention are those proteins that differ from a disclosed protein as the result of deletion or insertion of one or more amino acids in a native sequence.

As used herein, “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100.

The “Pfam” database is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

A “Pfam domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pfam domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected.

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software, a current version of which is provided in the appended computer listing. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are also included in the appended computer listing.

Version 19.0 of the HMMER software and Pfam databases were used to identify known domains in the proteins corresponding to amino acid sequence of SEQ ID NO: 340 through SEQ ID NO: 678. All DNA encoding proteins that have scores higher than the gathering cutoff disclosed in Table 23 by Pfam analysis disclosed herein can be used in recombinant DNA of the plant cells of this invention, e.g. for selecting transgenic plants having enhanced agronomic traits. The relevant Pfams modules for use in this invention, as more specifically disclosed below, are bZIP_{—}1, AOX, DUF902::DUF906. LRRNT_{—}2::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::LRR_{—}1::Pkinase, ABC_tran::ABC2_membrane::PDR_CDR::ABC_tran::ABC2_membrane, Redoxin, RNase_PH::RNase_PH_C, AAA, GFO_IDH_MocA::GFO_IDH_MocA_C, GRAS, Metallophos, Ribosomal_L18p, Sugar_tr, CDC48_N::AAA::AAA, Pkinase, PAS_{—}3::PAS_{—}3::Pkinase, CRAL_TRIO_N::CRAL_TRIO, p450, RRM_{—}1::RRM_{—}1, SRF-TF, G-alpha, TPR_{—}1::TPR_{—}1, FAE1_CUT1_RppA::ACP_syn_III_C, Globin::FAD_binding_{—}6::NAD_binding_{—}1, TPR_{—}1::TPR_{—}2, IF4E, F-box::LRR_{—}2, FBPase, LRR_{—}2::LRR_{—}1::LRR_{—}1::LRR_{—}1, HSF_DNA-bind, Dehydrin, TP_methylase, Response_reg::Myb_DNA-binding, KNOX1::KNOX2::ELK::Homeobox, Catalase, GTP_EFTU::GTP_EFTU_D2::GTP_EFTU_D3, TPR_{—}1::TPR_::TPR_{—}1::TPR_{—}1, ADH_zinc_N, Globin, CS, GH3, HLH, Ribonuclease_T2, TPR_{—}1::TPR_{—}1::TPR_::U-box, Dicty_CAR, Cyclin_N::Cyclin_C, MFS_, Acid_phosphat_A, Methyltransf_{—}7, TPR_{—}1::TPR_{—}1::TPR_{—}2, IBN_N, polyprenyl_synt, AhpC-TSA, Oxidored_FMN, Hydrolase, DS, Response_reg::CCT, Aa_trans, peroxidase, E1-E2_ATPase, F-box::Tub, Response_reg. Rho_GD1, E2F_TDP, 14-3-3, AT_hook::AT_hook::AT_hook::AT_hook::YDG_SRA::Pre-SET::SET, Tub, KOW::eIF-5a, MtN3_slv::MtN3_slv, GTP_EFTU, UQ_con, MAT1, E2F_TDP::E2F_TDP, HEAT::HEAT::HEAT::FAT::PI3_PI4_kinase::FATC, HMG_CoA_synt_N::HMG_CoA_synt_C, TAP42, DEAD::Helicase_C::DSHCT, NDK, Clp_N::Clp_N::AAA::AAA_{—}2, Cyclin_N, OPT, Orn_Arg_deC_N::Orn_DAP_Arg_deC, PAS::Pkinase, FtsH_ext::AAA::Peptidase_M41, Wzy_C, Mlo, AP2::B3, SET, FKBP_C::FKBP_C::FKBP_C::TPR_{—}1::TPR_{—}1, TPR_{—}2::TPR_{—}1::TPR_::TPR_{—}2::TPR_{—}1::TPR_{—}1::TPR_{—}1::TPR_{—}1::TPR_{—}1, Pyridoxal_deC, RNase_PH, RB_A::RB_B, WD40::WD40::WD40::WD40::WD40::WD40, SNF2_N::Helicase_C, Aminotran_{—}1_{—}2, Gemini_AL1::Gemini_AL1_M, Hexapep::Hexapep::Hexapep::Hexapep, AP2::AP2, Abbhydrolase_{—}1, PAS_{—}2::GAF::Phytochrome::PAS::PAS::HisKA::HATPase_c, Cystatin::Cystatin, Pfam module annotation, Cystatin, F-box::FBA_{—}1, 2OG-FeII_Oxy, FA_desaturase, HSP20, FBPase_glpX, E1-E2_ATPase::Hydrolase. Mito_carr::Mito_carr::Mito_carr, Cellulose_synt, Linker_histone::AT_hook::AT_hook::AT_hook::AT_hook, UPF0016::UPF0016, GD1, Glyco_hydro_{—}32N::Glyco_hydro_{—}32C, TPR_{—}1::TPR_{—}1::TPR_{—}2::U-box, ADH_N::ADH_zinc_N, GDA1_CD39, MIP, CRAL_TRIO, TPR_{—}1::TPR_{—}1::TPR_{—}1::TPR_{—}1::TPR_{—}1::TPR_{—}1::TPR_{—}1::TPR_{—}1, LEA_{—}4::LEA_{—}4, Carb_anhydrase, PTR2, Cu_bind_like, HD-ZIP_N::Homeobox::HALZ, eIF-5a, Asp, S1::S1::S1, SAM_decarbox, WD40::WD40, Citrate_synt, SRF-TF::K-box, HSP9_HSP2, PI3_PI4_kinase, Ferritin, Xan_ur_permease, Myb_DNA-binding::Myb_DNA-binding, zf-NF-X1::zf-NF-X1::zf-NF-X1::zf-NF-X1::zf-NF-X1, AP2, and Myb_DNA-binding.

As used herein “promoter” means regulatory DNA for initializing transcription. A “plant promoter” is a promoter capable of initiating transcription in plant cells whether or not its origin is a plant cell. e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that expressed a protein that impart an enhanced trait. A control plant is to identify and select a transgenic plant that has an enhance trait. A suitable control plant can be a nontransgenic plant of the parental line used to generate a transgenic plant, i.e. devoid of recombinant DNA. A suitable control plant may in some cases be a progeny of a hemizygous transgenic plant line that is does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tonnes per acre, tons per acre, kilo per hectare. For example, maize yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as tocopherol, protein and starch, or oil particular oil components as may be manifest by alterations in the ratios of seed components.

A subset of the nucleic molecules of this invention includes fragments of the disclosed recombinant DNA consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO:1 through SEQ ID NO: 339, and find use, for example as probes and primers for detection of the polynucleotides of the present invention.

DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasnmids of Agrobacterium tumefaciens, caulimovirus promoters such as the cauliflower mosaic virus. For instance, see U.S. Pat. Nos. 5,858,742 and 5,322,938, which disclose versions of the constitutive promoter derived from cauliflower mosaic virus (CaMV35S), U.S. Pat. No. 5,641,876, which discloses a rice actin promoter, U.S. Patent Application Publication 2002/0192813A1, which discloses 5′,3′ and intron elements useful in the design of effective plant expression vectors, U.S. patent application Ser. No. 09/757,089, which discloses a maize chloroplast aldolase promoter, U.S. patent application Ser. No. 08/706,946, which discloses a rice glutelin promoter, U.S. patent application Ser. No. 09/757,089, which discloses a maize aldolase (FDA) promoter, and U.S. Patent Application Ser. No. 60/310,370, which discloses a maize nicotinamide synthase promoter, all of which are incorporated herein by reference. These and numerous other promoters that function in plant cells are known to those skilled in the art and available for use in recombinant polynucleotides of the present invention to provide for expression of desired genes in transgenic plant cells.

In other aspects of the invention, preferential expression in plant green tissues is desired. Promoters of interest for such uses include those from genes such as Arabidopsis thaliana ribulose-1,5-bisphosphate carboxylase (Rubisco) small subunit (Fischhoff et at (1992) Plant Mol Biol. 20:81-93), aldolase and pyruvate orthophosphate dikinase (PPDK) (Taniguchi et al. (2000) Plant Cell Physiol. 41(1):42-48).

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene.

In other aspects of the invention, sufficient expression in plant seed tissues is desired to affect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2): 157-166), globulin 1 (Belanger et al (1991) Genetics 129:863-872), glutelin 1 (Russell (1997) supra), and peroxiredoxin antioxidant (Per1) (Stacy et al. (1996) Plant Mot Biol. 31(6):1205-1216).

Recombinant DNA constructs prepared in accordance with the invention will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, for example disclosed in U.S. Pat. No. 6,090,627, incorporated herein by reference; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al (MGG (1987) 210:437-442).

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent Application publication 2003/0083480 A1 also for imparting glyphosate tolerance; dicamba monooxygenase disclosed in U.S. Patent Application publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260, all of said U.S. patents and patent application Publications are incorporated herein by reference. Molecules and methods for imparting insect/nematodelvirus resistance are disclosed in U.S. Pat. Nos. 5,250,515; 5,880,275; 6,506,599; 5,986,175 and U.S. Patent Application Publication 2003/0150017 A1, all of which are incorporated herein by reference.

Plant Cell Transformation Methods Numerous methods for transforming plant cells with recombinant DNA are known in the an and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,463,174 (canola); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly. i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants including cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, hypocotyls, calli, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, hypocotyls, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into a first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV) spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047, all of which are incorporated herein by reference. Selectable markers which provide an ability to visually identify transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue, and the plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seed

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 2 provides a list of protein encoding DNA (“genes”) that are useful as recombinant DNA for production of transgenic plants with enhanced agronomic trait.
Column headings in Table 2 refer to the following information:
“PEP SEQ ID NO” refers to a particular amino acid sequence in the Sequence Listing “PHE ID” refers to an arbitrary number used to identify a particular recombinant DNA corresponding to the translated protein encoded by the polynucleotide.
“NUC SEQ ID NO” refers to a particular nucleic acid sequence in the Sequence Listing which defines a polynucleotide used in a recombinant DNA of this invention.
“GENE NAME” refers to a common name for the recombinant DNA.
“CODING SEQUENCE” refers to peptide coding segments of the corresponding recombinant DNA.
“SPECIES” refers to the organism from which the recombinant DNA was derived.

TABLE 2

| PEP | | NUC | | | |
|---|---|---|---|---|---|
| SEQ ID | Phe | SEQ ID | Gene | CODING | |
| NO | ID | NO | Name | SEQUENCE | Species |
| 340 | PHE0000001 | 1 | maize cellulose synthase | 113-3061 | Zea mays |
| | | | (eskimo 2) | | |
| 341 | PHE0000006 | 2 | Arabidopsis RA V2/G9 | 81-1136 | Arabidopsis thaliana |
| 342 | PHE0000007 | 3 | rice G9-like 1 | 336-1430 | Oryza sativa |
| 343 | PHE0000008 | 4 | rice G9-like 2 | 572-1522 | Oryza sativa |
| 344 | PHE0000010 | 5 | rice G975 | 201-283.516-1161 | Oryza sativa |
| 345 | PHE0000278 | 6 | corn G975 | 41-679 | Zea mays |
| 346 | PHE0000011 | 7 | corn Glossy 15 | 385-1722 | Zea mays |
| 347 | PHE0000012 | 8 | corn aquaporin RS81 | 1-747 | Zea mays |
| 348 | PHE0000014 | 9 | rice cycD2 | 13-324, 623-709, 813- | Oryza sativa |
| | | | | 911, 1003-1204, 1314- | |
| | | | | 1438, 1529-1774 | |
| 349 | PHE0000215 | 10 | invW | 1108-1489, 1813-2684, | Oryza sativa |
| | | | | 6105-6266, 6417-6658. | |
| 350 | PHE0000015 | 11 | rice GCR1 | 312-500, 1123-1154, 1384- | Oryza sativa |
| | | | | 1553, 2048-2163, 2724- | |
| | | | | 2825, 2946-3002, 3331- | |
| | | | | 3474, 3930-4000, 4118-4223 | |
| 351 | PHE0000016 | 12 | corn Knotted1 | 181-1257 | Zea mays |
| 352 | PHE0000018 | 13 | corn AAA-ATPase 2 | 104-2533 | Zea mays |
| 353 | PHE0000019 | 14 | rice AOX1b (alternative | 4531-4851, 5011-5139, | Oryza sativa |
| | | | oxidase) | 6072-6560, 6663-6722 | |
| 354 | PHE0000020 | 15 | Emericella nidulans alxA | 2189-2442, 2492-2783, | Emericella nidulans |
| | | | | 2843-3352 | |
| 355 | PHE0000022 | 16 | corn AAP6-like | 96-1547 | Zea mays |
| 356 | PHE0000024 | 17 | corn unknown protein | 441-2390 | Zea mays |
| 357 | PHE0000025 | 18 | corn GRF1-like protein | 55-1470 | Zea mays |
| 358 | PHE0000026 | 19 | rice GRF1 | 193-1380 | Oryza sativa |
| 359 | PHE0000227 | 20 | soy omega-3 fatty acid | 138-1496 | Glycine max |
| | | | desaturase | | |
| 360 | PHE0000258 | 21 | AtFAD7 | 132-1472 | Arabidopsis thaliana |
| 361 | PHE0000259 | 22 | AtFAD8 | 61-1368 | Arabidopsis thaliana |
| 362 | PHE0000049 | 23 | rice phyA with corn phyC | 4626-6690, 6913-7729, | Oryza sativa |
| | | | intron 1 | 8011-8307, 8410-8617 | |
| 363 | PHE0000027 | 24 | sorghum phyA with corn | 238-3633 | Sorghum bicolor |
| | | | phyC intron 1 | | |
| 364 | PHE0000028 | 25 | rice phyB with corn phyC | 67-3582 | Oryza sativa |
| | | | intron 1 | | |
| 365 | PHE0000029 | 26 | sorghum phyB with corn | 429-2640, 3333-4140, | Sorghum bicolor |
| | | | phyC intron 1 | 5819-6112, 7491-7713 | |
| 366 | PHE0000030 | 27 | rice phyC with corn phyC | 1036-3100, 3205-4021, | Oryza sativa |
| | | | intron 1 | 4418-4711, 5272-5509 | |
| 367 | PHE0000031 | 28 | sorghum phyC with corn | 303-3710 | Sorghum bicolor |
| | | | phyC intron 1 | | |
| 368 | PHE0000032 | 29 | rice PF1 | 35-676 | Oryza sativa |
| 369 | PHE0000033 | 30 | rice GT2 | 58-2271 | Oryza sativa |
| 370 | PHE0000034 | 31 | Synechocystis biliverdin | 9-992 | Synechocystis sp. |
| | | | reductase | | PCC 6803 |
| 371 | PHE0000038 | 32 | corn cycD2.1 | 125-1156 | Zea mays |
| 372 | PHE0000039 | 33 | corn nph1 | 415-3150 | Zea mays |
| 373 | PHE0000040 | 34 | corn hemoglobin 1 | 172-669 | Zea mays |
| 374 | PHE0000043 | 35 | rice cyclin 2 | 148-1407 | Oryza sativa |
| 375 | PHE0000044 | 36 | rice cycC | 97-870 | Oryza sativa |
| 376 | PHE0000045 | 37 | rice cycB2 | 74-1336 | Oryza sativa |
| 377 | PHE0000046 | 38 | rice cycA1 | 97-1623 | Oryza sativa |
| 378 | PHE0000047 | 39 | rice cycB5 | 292-361, 1019-1347, | Oryza sativa |
| | | | | 1447-1572, 1657-1908, | |
| | | | | 2059-2217, 2315-2493, | |
| | | | | 3276-3432 | |
| 379 | PHE0000244 | 40 | corn SVP-like | 177-860 | Zea mays |
| 380 | PHE0000245 | 41 | corn SVP-like | 93-791 | Zea mays |
| 381 | PHE0000246 | 42 | soy SVP-like | 96-713 | Glycine max |
| 382 | PHE0000247 | 43 | soy jointless-like | 60-674 | Glycine max |
| 383 | PHE0000106 | 44 | corn cycA1 | 107-1633 | Zea mays |
| 384 | PHE0000050 | 45 | corn cycA2 | 107-1222 | Zea mays |
| 385 | PHE0000051 | 46 | corn cycB2 | 137-1408 | Zea mays |
| 386 | PHE0000052 | 47 | corn cycB5 | 82-1518 | Zea mays |
| 387 | PHE0000382 | 48 | LIB3279-180-C9_FLI- | 114-1385 | Zea mays |
| | | | maize cyclin III | | |
| 388 | PHE0000053 | 49 | corn cycB4 | 254-1579 | Zea mays |
| 389 | PHE0000054 | 50 | corn cycD3.2 | 220-1380 | Zea mays |
| 390 | PHE0000055 | 51 | corn cycDx.1 | 218-1180 | Zea mays |
| 391 | PHE0000056 | 52 | corn cycD1.1 | 288-1334 | Zea mays |
| 392 | PHE0000057 | 53 | corn mt NDK- | 60-725 | Zea mays |
| | | | L1B189022Q1E1E9 | | |
| 393 | PHE0000058 | 54 | corn cp NDK- | 103-816 | Zea mays |
| | | | 700479629 | | |
| 394 | PHE0000059 | 55 | corn NDK- | 49-495 | Zea mays |
| | | | L1B3597020Q1K6C3 | | |
| 395 | PHE0000060 | 56 | corn NDK-700241377 | 162-608 | Zea mays |
| 396 | PHE0000062 | 57 | sRAD54-with NLS | 437-3556 | Synechocystis sp. |
| | | | | | PCC 6803 |
| 397 | PHE0000063 | 58 | T4 endonuclease VII | 603-1-148 | coliphage T4 |
| | | | (gp49)-with NLS | | |
| 398 | PHE0000064 | 59 | corn NDPK-fC- | 91-624 | Zea mays |
| | | | zmemL1B3957015Q1K6 | | |
| | | | H6 | | |
| 399 | PHE0000065 | 60 | TOR1 | 302-7714 | Saccharomyces |
| | | | | | cerevisiae |
| 400 | PHE0000292 | 61 | corn eIF-5A | 85-564 | Zea mays |
| 401 | PHE0000067 | 62 | yeast eIF-5A | 569-1042 | Saccharomyces |
| | | | | | cerevisiae |
| 402 | PHE0000068 | 63 | yeast deoxyhypusine | 173-1336 | Saccharomyces |
| | | | synthase | | cerevisiae |
| 403 | PHE0000069 | 64 | yeast L5 | 987-1880 | Saccharomyces |
| | | | | | cerevisiae |
| 404 | PHE0000070 | 65 | yeast ornithine | 576-1976 | Saccharomyces |
| | | | decarboxylase | | cerevisiae |
| 405 | PHE0000071 | 66 | rice exportin 4-like | 501-750, 1257-1417, | Oryza sativa |
| | | | | 1735-1800, 3104-3218, | |
| | | | | 3318-3427, 3525-3620, | |
| | | | | 7587-7744, 7828-7915, | |
| | | | | 8565-8669, 8774-8878, | |
| | | | | 9421-9450, 9544-9656, | |
| | | | | 9732-9819, 9961-10180, | |
| | | | | 11034-11164, 12058- 12204, | |
| | | | | 12770-12898, 12975-13073, | |
| | | | | 13221-13259, 14674-14823 | |
| 406 | PHE0000072 | 67 | yeast S- | 415-1605 | Saccharomyces |
| | | | adenosylmethionine | | cerevisiae |
| | | | decarboxylase | | |
| 407 | PHE0000073 | 68 | corn S- | 268-1365 | Zea mays |
| | | | adenosylmethionine | | |
| | | | decarboxylase 1 | | |
| 408 | PHE0000074 | 69 | corn S- | 581-1780 | Zea mays |
| | | | adenosylmethionine | | |
| | | | decarboxylase 2 | | |
| 409 | PHE0000075 | 70 | retinoblastoma-related | 37-2634 | Zea mays |
| | | | protein 1 | | |
| 410 | PHE0000076 | 71 | C1 protein | 49-843 | Wheat dwarf virus |
| 411 | PHE0000077 | 72 | yeast flavohemoglobin- | 1695-2894 | Saccharomyces |
| | | | mitochondrial | | cerevisiae |
| 412 | PHE0000009 | 73 | Arabidopsis G975 | 58-654 | Arabidopsis thaliana |
| 413 | PHE0000079 | 74 | CUT1 | 372-1082, 1176-1946 | Oryza sativa |
| 414 | PHE0000082 | 75 | corn cycB3 | 88-1425 | Zea mays |
| 415 | PHE0000083 | 76 | PDR5 | 1552-6087 | Saccharomyces |
| | | | | | cerevisiae |
| 416 | PHE0000084 | 77 | rice cyclin H | 235-1227 | Oryza sativa |
| 417 | PHE0000085 | 78 | rice cdc2+/CDC28- | 173-1447 | Oryza sativa |
| | | | related protein kinase | | |
| 418 | PHE0000086 | 79 | Cdk-activating kinase 1 | 14-1240 | Glycine max |
| 419 | PHE0000089 | 80 | CHL1 | 85-1857 | Arabidopsis thaliana |
| 420 | PHE0000090 | 81 | NTR1 | 144-1898 | Oryza sativa |
| 421 | PHE0000091 | 82 | Zm SET domain 2 | 101-1009 | Zea mays |
| 422 | PHE0000092 | 83 | Zm SET domain 1 | 528-1544 | Zea mays |
| 423 | PHE0000095 | 84 | HSF1 | 1017-3518 | Saccharomyces |
| | | | | | cerevisiae |
| 424 | PHE0000096 | 85 | Zm HSP101 | 436-1773, 1878-2159, | Zea mays |
| | | | | 2281-2621, 2711-2990, | |
| | | | | 3079-3276, 3371-3670 | |
| 425 | PHE0000098 | 86 | E. coli clpB | 557-3130 | Escherichia coli |
| 426 | PHE0000099 | 87 | Synechocystis clpB | 316-2931 | Synechocystis sp. |
| | | | | | PCC 6803 |
| 427 | PHE0000100 | 88 | Xylella clpB | 187-2769 | Xylella fastidiosa |
| 428 | PHE0000101 | 89 | corn cycD3.1 | 250-1422 | Zea mays |
| 429 | PHE0000102 | 90 | AnFPPS (farnesyl- | 146-1186 | Emericella nidulans |
| | | | pyrophosphate | | |
| | | | synthetase) | | |
| 430 | PHE0000103 | 91 | OsFPPS | 42-1103 | Oryza sativa |
| 431 | PHE0000104 | 92 | 700331819_FLI-corn | 313-1377 | Zea mays |
| | | | FPPS 2 | | |
| 432 | PHE0000105 | 93 | corn cycD1.2 | 229-1275 | Zea mays |
| 433 | PHE0000107 | 94 | corn cycD1.3 | 206-1252 | Zea mays |
| 434 | PHE0000108 | 95 | ASH1 | 61-801 | Arabidopsis thaliana |
| 435 | PHE0000109 | 96 | rice ASH1-like 1 | 136-1008 | Oryza sativa |
| 436 | PHE0000110 | 97 | rice MtN2-like | 425-464, 546-582, 672- | Oryza sativa |
| | | | | 783, 812-898, 988-1149, | |
| | | | | 1556-1675, 1776-1952 | |
| 437 | PHE0000111 | 98 | PAS domain kinase | 358-2613 | Zea mays |
| 438 | PHE0000114 | 99 | Su(var) 3-9-like | 71-814 | Zea mays |
| 439 | PHE0000115 | 100 | Receiver domain (RR3- | 277-1002 | Zea mays |
| | | | like) 7 | | |
| 440 | PHE0000116 | 101 | Receiver domain | 188-2245 | Zea mays |
| | | | (ARR2-like) 1 | | |
| 441 | PHE0000117 | 102 | Receiver domain (TOC1- | 112-2238 | Zea mays |
| | | | like) 2 | | |
| 442 | PHE0000118 | 103 | Receiver domain (TOC1- | 84-1976 | Zea mays |
| | | | like) 3 | | |
| 443 | PHE0000119 | 104 | Receiver domain (ARR2- | 39-1931 | Zea mays |
| | | | like) 4 | | |
| 444 | PHE0000120 | 105 | Receiver domain (RR11- | 61-1812 | Zea mays |
| | | | like) 5 | | |
| 445 | PHE0000121 | 106 | Receiver domain (RR3- | 391-1116 | Zea mays |
| | | | like) 6 | | |
| 446 | PHE0000122 | 107 | Receiver domain (RR3- | 335-1066 | Zea mays |
| | | | like) 8 | | |
| 447 | PHE0000123 | 108 | Receiver domain 9 | 55-759 | Zea mays |
| 448 | PHE0000124 | 109 | ZmRR2 | 154-624 | Zea mays |
| 449 | PHE0000125 | 110 | Receiver domain (TOC1- | 374-722, 791-2019 | Zea mays |
| | | | like) 10 | | |
| 450 | PHE0000126 | 111 | corn HY5-like | 32-541 | Zea mays |
| 451 | PHE0000127 | 112 | scarecrow 1 (PAT1-like) | 295-1929 | Zea mays |
| 452 | PHE0000128 | 113 | scarecrow 2 | 153-1934 | Zea mays |
| 453 | PHE0000133 | 114 | G protein b subunit | 90-1229 | Zea mays |
| 454 | PHE0000152 | 115 | 14-3-3-like protein 2 | 85-861 | Glycine max |
| 455 | PHE0000153 | 116 | 14-3-3-like protein D | 42-824 | Glycine max |
| 456 | PHE0000154 | 117 | 14-3-3 protein 1 | 49-834 | Glycine max |
| 457 | PHE0000155 | 118 | Rice FAP1-like protein | 654-1862, 2310-2426, | Oryza sativa |
| | | | | 3407-3492, 3590-3752, | |
| | | | | 3845-3890, 4476-4522, | |
| | | | | 4985-5191, 5306-5392, | |
| | | | | 5473-5640 | |
| 458 | PHE0000156 | 119 | rice TAP42-like | 199-1338 | Oryza sativa |
| 459 | PHE0000158 | 120 | BMH1 | 79-882 | Saccharomyces |
| | | | | | cerevisiae |
| 460 | PHE0000159 | 121 | rice chloroplastic | 41-1261 | Oryza sativa |
| | | | fructose-1,6- | | |
| | | | bisphosphatase | | |
| 461 | PHE0000160 | 122 | E. coli fructose-1,6- | 208-1206 | Escherichia coli |
| | | | bisphosphatase | | |
| 462 | PHE0000161 | 123 | Synechocystis fructose- | 1-1164 | Synechocystis sp. |
| | | | 1,6-bisphosphatase F-1 | | PCC 6803 |
| 463 | PHE0000162 | 124 | Synechocystis fructose- | 480-1523 | Synechocystis sp. |
| | | | 1,6-bisphosphatase F-11 | | PCC 6803 |
| 464 | PHE0000164 | 125 | Yeast RPT5 | 883-2187 | Saccharomyces |
| | | | | | cerevisiae |
| 465 | PHE0000165 | 126 | Yeast RRP5 | 331-5520 | Saccharomyces |
| | | | | | cerevisiae |
| 466 | PHE0000166 | 127 | Rice CBP-like gene | 277-436, 479-1524, 1790- | Oryza sativa |
| | | | | 2065, 2150-2425, 3134- | |
| | | | | 3262, 3380-3580, 3683- | |
| | | | | 3825, 3905-4190, 4294- | |
| | | | | 4433, 4711-4789, 4874- | |
| | | | | 4929, 5754-5946 | |
| 467 | PHE0000167 | 128 | rice BAB09754 | 616-903, 1848-1940, 2046- | Oryza sativa |
| | | | | 2165, 2254-2355, 2443- | |
| | | | | 2693, 2849-2994, 3165- | |
| | | | | 3363, 3475-4141, 4438- | |
| | | | | 4770, 5028-5309 | |
| 468 | PHE0000168 | 129 | L1B3061-001-H7_FL1 | 309-1037 | Zea mays |
| 469 | PHE0000169 | 130 | maize p23 | 106-708 | Zea mays |
| 470 | PHE0000170 | 131 | maize cyclophilin | 99-1757 | Zea mays |
| 471 | PHE0000172 | 132 | yeast SIT1 | 361-2130 | Saccharomyces |
| | | | | | cerevisiae |
| 472 | PHE0000173 | 133 | yeast CNS1 | 762-1919 | Saccharomyces |
| | | | | | cerevisiae |
| 473 | PHE0000176 | 134 | RNAse S | 85-771 | Zea mays |
| 474 | PHE0000177 | 135 | maize ecto-apyrase | 210-2312 | Zea mays |
| 475 | PHE0000178 | 136 | PHO5 | 1-1404 | Saccharomyces |
| | | | | | cerevisiae |
| 476 | PHE0000179 | 137 | high affinity phosphate | 105-1703 | Glycine max |
| | | | translocator | | |
| 477 | PHE0000180 | 138 | high affinity phosphate | 128-1750 | Zea mays |
| | | | translocator | | |
| 478 | PH60000181 | 139 | Xylella citrate synthase | 256-1545 | Xylella fastidiosa |
| 479 | PHE0000182 | 140 | E. coli citrate synthase | 309-1592 | Escherichia coli |
| 480 | PHE0000183 | 141 | rice citrate synthase | 105-1523 | Oryza sativa |
| 481 | PHE0000184 | 142 | citrate synthase | 56-1564 | Zea mays |
| 482 | PHE0000185 | 143 | citrate synthase | 153-1691 | Glycine max |
| 483 | PHE0000186 | 144 | maize ferritin 2 | 3-758 | Zea mays |
| 484 | PHE0000187 | 145 | maize ferritin 1 | 34-795 | Zea mays |
| 485 | PHE0000188 | 146 | E. coli cytoplasmic | 245-742 | Escherichia coli |
| | | | ferritin | | |
| 486 | PHE0000190 | 147 | corn LEA3 | 171-755 | Zea mays |
| 487 | PHE0000192 | 148 | soy HSF | 23-1114 | Glycine max |
| 488 | PHE0000193 | 149 | soy HSF | 93-992 | Glycine max |
| 489 | PHE0000204 | 150 | deoxyhypusine synthase | 26-1129 | Glycine max |
| 490 | PHE0000219 | 151 | thylakoid carbonic | 62-994 | Chlamydomonas |
| | | | anhydrase, cah3 | | reinhardtii |
| 491 | PHE0000216 | 152 | thylakoid carbonic | 49-843 | Nostoc PCC7120 |
| | | | anhydrase, ecaA | | |
| 492 | PHE0000217 | 153 | Chlamydomonas | 156-1232 | Chlamydomonas |
| | | | reinhardtii envelope | | reinhardtii |
| | | | protein LIP-36G1 | | |
| 493 | PHE0000218 | 154 | psbO transit | 271-1674 | Synechococcus sp. |
| | | | peptide::Synechococcus | | PCC 7942 |
| | | | sp. PCC 7942 ictB | | |
| 494 | PHE0000220 | 155 | corn RNase PH | 86-805 | Zea mays |
| 495 | PHE0000221 | 156 | SKI2 | 1351-5211 | Saccharomyces |
| | | | | | cerevisiae |
| 496 | PHE0000222 | 157 | SKI3 | 793-5091 | Saccharomyces |
| | | | | | cerevisiae |
| 497 | PHE0000223 | 158 | SKI4 | 323-1201 | Saccharomyces |
| | | | | | cerevisiae |
| 498 | PHE0000224 | 159 | SKI6 | 1007-1747 | Saccharomyces |
| | | | | | cerevisiae |
| 499 | PHE0000225 | 160 | SKI7 | 279-2519 | Saccharomyces |
| | | | | | cerevisiae |
| 500 | PHE0000226 | 161 | rice SKI7-like | 464-884, 1132-1287, 2103- | Oryza sativa |
| | | | | 2252, 2353-2487, 2957- | |
| | | | | 3288, 3399-3509, 3596- | |
| | | | | 4095, 4350-4518, 4783- | |
| | | | | 5022, 5097-5228, 5315-5449 | |
| 501 | PHE0000228 | 162 | Synechocystis cobA w cp | 70-801 | Synechocystis sp. |
| | | | transit peptide | | PCC 6803 |
| 502 | PHE0000229 | 163 | Xylella tetrapyrrole | 1-774 | Xylella fastidiosa |
| | | | methylase with transit | | |
| | | | peptide | | |
| 503 | PHE0000230 | 164 | maize uroporphyrinogen | 15-1286 | Zea mays |
| | | | III methyltransferase | | |
| 504 | PHE0000231 | 165 | nucellin-like protein | 122-1594 | Zea mays |
| 505 | PHE0000232 | 166 | nucellin-like protein | 76-1605 | Zea mays |
| 506 | PHE0000233 | 167 | nucellin-like protein | 195-1628 | Zea mays |
| 507 | PHE0000234 | 168 | soy LEA protein | 6-704 | Glycine max |
| 508 | PHE0000235 | 169 | dehydrin-like protein | 33-710 | Glycine max |
| 509 | PHE0000237 | 170 | dehydrin 3 | 84-584 | Zea mays |
| 510 | PHE0000238 | 171 | probable lipase | 98-967 | Zea mays |
| 511 | PHE0000239 | 172 | yeast GRE 1 | 1024-1527 | Saccharomyces |
| | | | | | cerevisiae |
| 512 | PHE0000240 | 173 | yeast STF2 | 683-934 | Saccharomyces |
| | | | | | cerevisiae |
| 513 | PHE0000241 | 174 | yeast SIP18 | 376-855 | Saccharomyces |
| | | | | | cerevisiae |
| 514 | PHE0000242 | 175 | yeast YBM6 | 744-1130 | Saccharomyces |
| | | | | | cerevisiae |
| 515 | PHE0000243 | 176 | yeast HSP12 | 282-611 | Saccharomyces |
| | | | | | cerevisiae |
| 516 | PHE0000249 | 177 | corn allene oxide | 111-1556 | Zea mays |
| | | | synthase | | |
| 517 | PHE0000250 | 178 | corn COI1-like | 139-1911 | Zea mays |
| 518 | PHE0000251 | 179 | corn TIR1-like | 113-1906 | Zea mays |
| 519 | PHE0000252 | 180 | corn COI1-like | 130-1923 | Zea mays |
| 520 | PHE0000253 | 181 | COI1-like | 389-2368 | Zea mays |
| 521 | PHE0000254 | 182 | F-box protein | 123-1304 | Glycine max |
| 522 | PHE0000255 | 183 | F-box protein | 228-1916 | Glycine max |
| 523 | PHE0000256 | 184 | corn 1- | 61-1011 | Zea mays |
| | | | aminocyclopropane-1- | | |
| | | | carboxylate oxidase | | |
| 524 | PHE0000257 | 185 | rice 1- | 2-1465 | Oryza sativa |
| | | | aminocyc lopropane-1 | | |
| | | | carboxylate synthasc | | |
| 525 | PHE0000260 | 186 | S52650-Synechocystis | 643-1719 | Synechocystis sp. |
| | | | desB | | PCC 6803 |
| 526 | PHE0000261 | 187 | yeast glutamate | 33-1790 | Saccharomyces |
| | | | decarboxylase | | cerevisiae |
| 527 | PHE0000262 | 188 | cytochrome P450-like | 29-1495 | Zea mays |
| | | | protein | | |
| 528 | PHE0000263 | 189 | cytochrome P450 | 141-1637 | Zea mays |
| 529 | PHE0000264 | 190 | cytochrome P450-like | 104-1657 | Zea mays |
| 530 | PHE0000265 | 191 | CYP90 protein | 81-1589 | Zea mays |
| 531 | PHE0000266 | 192 | cytochrome P450 | 92-1648 | Zea mays |
| | | | DWARF3 | | |
| 532 | PHE0000267 | 193 | cytochrome P450 | 134-1543 | Zea mays |
| 533 | PHE0000268 | 194 | rice receptor protein | 183-476, 706-735, | Oryza sativa |
| | | | kinase | 2796-6734 | |
| 534 | PHE0000269 | 195 | soy E2F-like | 80-1117 | Glycine max |
| 535 | PHE0000270 | 196 | nuclear matrix constituent | 243-3371 | Zea mays |
| | | | protein | | |
| 536 | PHE0000271 | 197 | OsE2F1 | 93-1403 | Oryza sativa |
| 537 | PHE0000272 | 198 | corn GCR1 | 74-1036 | Zea mays |
| 538 | PHE0000273 | 199 | soy mlo-like | 15-1532 | Glycine max |
| 539 | PHE0000274 | 200 | soy mlo-like | 48-1841 | Glycine max |
| 540 | PHE0000275 | 201 | rice G alpha 1 | 106-1248 | Oryza sativa |
| 541 | PHE0000276 | 202 | soy G-gamma subunit | 210-536 | Glycine max |
| 542 | PHE0000277 | 203 | wheat G28-like | 65-877 | Triticurm aestivum |
| 543 | PHE0000279 | 204 | sorghum proline | 16-1341 | Sorghum bicolor |
| | | | permease | | |
| 544 | PHE0000280 | 205 | rice AA transporter | 61-1485 | Oryza sativa |
| 545 | PHE0000282 | 206 | SET-domain protein-like | 478-3045 | Zea mays |
| 546 | PHE0000283 | 207 | scarecrow 6 | 520-2145 | Zea mays |
| 547 | PHE0000284 | 208 | menage a trois-like | 164-745 | Zea mays |
| 548 | PHE0000286 | 209 | oryzacystatin | 108-527 | Oryza sativa |
| 549 | PHE0000287 | 210 | Similar to cysteine | 18-767 | Oryza sativa |
| | | | proteinase inhibitor | | |
| 550 | PHE0000288 | 211 | cysteine proteinase | 135-461 | Sorghum bicolor |
| | | | inhibitor | | |
| 551 | PHE0000289 | 212 | Zm-GRF1 (GA | 96-1202 | Zea mays |
| | | | responsive factor) | | |
| 552 | PHE0000290 | 213 | ZmSE001-like | 253-2115 | Zea mays |
| 553 | PHE0000291 | 214 | deoxyhypusine synthase | 54-1163 | Zea mays |
| 554 | PHE0000293 | 215 | gibberellin response | 131-2020 | Zea mays |
| | | | modulator | | |
| 555 | PHE0000294 | 216 | scarecrow-like protein | 266-1948 | Zea mays |
| 556 | PHE0000295 | 217 | ubiquitin-conjugating | 114-599 | Zea mays |
| | | | enzyme-like protein | | |
| 557 | PHE0000296 | 218 | unknown protein | 90-785 | Zea mays |
| | | | recognized by PF01169 | | |
| 558 | PHE0000297 | 219 | 26S protease regulatory | 57-1343 | Oryza sativa |
| | | | subunit 6A homolog | | |
| 559 | PHE0000298 | 220 | rice p23 co-chaperone | 68-706 | Oryza sativa |
| 560 | PHE0000299 | 221 | corn p23 co-chaperone | 71-565 | Zea mays |
| 561 | PHE0000300 | 222 | rice p23 co-chaperone | 124-642 | Oryza sativa |
| 562 | PHE0000301 | 223 | corn p23 co-chaperone | 90-617 | Zea mays |
| 563 | PHE0000302 | 224 | putative purple acid | 22-1038 | Oryza sativa |
| | | | phosphatase precursor | | |
| 564 | PHE0000303 | 225 | acid phosphatase type 5 | 143-1186 | Zea mays |
| 565 | PHE0000304 | 226 | aleurone ribonuclease | 47-814 | Oryza sativa |
| 566 | PHE0000305 | 227 | putative ribonuclease | 55-888 | Zea mays |
| 567 | PHE0000306 | 228 | S-like RNase | 15-770 | Zea mays |
| 568 | PHE0000307 | 229 | ribonuclease | 95-781 | Zea mays |
| 569 | PHE0000308 | 230 | helix-loop-helix protein | 202-756 | Zea mays |
| | | | (PIF3-like) | | |
| 570 | PHE0000309 | 231 | SKI4-like protein | 36-632 | Zea mays |
| 571 | PHE0000310 | 232 | putative 3 | 238-1098 | Zea mays |
| | GFI1 | | exoribonuclease | | |
| 572 | PHE0000311 | 233 | GFI4-c protein | 81-848 | Oryza sativa |
| 573 | PHE0000312 | 234 | 14-3-3-like protein | 6-785 | Oryza sativa |
| 574 | PHE0000313 | 235 | rice eIF-(iso)4F | 96-713 | Oryza sativa |
| 575 | PHE0000314 | 236 | rice eIF-4F | 46-726 | Oryza sativa |
| 576 | PHE0000315 | 237 | sorghum eIF-(iso)4F | 78-707 | Sorghum bicolor |
| 577 | PHE0000316 | 238 | sorghum eIF-4F | 9-668 | Sorghum bicolor |
| 578 | PHE0000317 | 239 | rice FIP37-like | 73-1128 | Oryza sativa |
| 579 | PHE0000318 | 240 | scarecrow 17 | 441-2102 | Zea mays |
| 580 | PHE0000322 | 241 | maize catalase-1 | 208-1683 | Zea mays |
| 581 | PHE0000323 | 242 | maize catalase-3 | 30-1511 | Zea mays |
| 582 | PHE0000324 | 243 | ascorbate peroxidase | 197-1063 | Zea mays |
| 583 | PHE0000325 | 244 | corn GD1 | 57-1397 | Zea mays |
| 584 | PHE0000326 | 245 | soy GD1 | 45-1418 | Glycine max |
| 585 | PHE0000327 | 246 | corn rho GD1 | 463-1203 | Zea mays |
| 586 | PHE0000328 | 247 | basic blue copper protein | 13-408 | Zea mays |
| 587 | PHE0000329 | 248 | plantacyanin | 109-489 | Zea mays |
| 588 | PHE0000330 | 249 | basic blue copper protein | 83-463 | Glycine max |
| 589 | PHE0000331 | 250 | Similar to blue copper | 323-868 | Zea mays |
| | | | protein precursor | | |
| 590 | PHE0000332 | 251 | lamin | 62-646 | Zea mays |
| 591 | PHE0000333 | 252 | fC-zmf1700551169a-allyl | 56-1105 | Zea mays |
| | | | alcohol dehydrogenase | | |
| 592 | PHE0000334 | 253 | allyl alcohol | 103-1128 | Glycine max |
| | | | dehydrogenase | | |
| 593 | PHE0000335 | 254 | allyl alcohol | 6-1079 | Zea mays |
| | | | dehydrogenase | | |
| 594 | PHE0000336 | 255 | quinone oxidoreductase | 47-1051 | Zea mays |
| 595 | PHE0000337 | 256 | E. nidulans cysA- | 384-1961 | Emericella nidulans |
| | | | AF029885 | | |
| 596 | PHE0000338 | 257 | BAA18167- | 801-1547 | Synechocystis sp. |
| | | | Synechocystis cysE | | PCC 6803 |
| 597 | PHE0000339 | 258 | Synechocystis thiol- | 36-638 | Synechocystis sp. |
| | | | specific antioxidant | | PCC 6803 |
| | | | protein-BAA10136 | | |
| 598 | PHE0000340 | 259 | yeast TSA2-NP_010741 | 108-698 | Saccharomyces |
| | | | | | cerevisiae |
| 599 | PHE0000341 | 260 | yeast mTPx-Z35825 | 730-1512 | Saccharomyces |
| | | | | | cerevisiae |
| 600 | PHE0000343 | 261 | yeast TPx III- | 657-1187 | Saccharomyces |
| | | | NP_013210 | | cerevisiae |
| 601 | PHE0000345 | 262 | soy putative 2-cys | 160-939 | Glycine max |
| | | | peroxiredox in | | |
| 602 | PHE0000346 | 263 | soy peroxiredoxin | 104-745 | Glycine max |
| 603 | PHE0000347 | 264 | heat shock protein 26, | 117-836 | Zea mays |
| | | | plastid-localized | | |
| 604 | PHE0000349 | 265 | heat shock protein | 112-735 | Zea mays |
| 605 | PHE0000350 | 266 | low molecular weight | 28-690 | Zea mays |
| | | | heat shock protein | | |
| 606 | PHE0000351 | 267 | 18 kDa heat shock protein | 103-597 | Zea mays |
| 607 | PHE0000352 | 268 | heat shock protein 16.9 | 229-690 | Zea mays |
| 608 | PHE0000353 | 269 | HSP21-like protein | 73-696 | Zea mays |
| 609 | PHE0000354 | 270 | Opt1p-NP_012323 | 508-2904 | Saccharomyces |
| | | | | | cerevisiae |
| 610 | PHE0000355 | 271 | SVCT2-like permease | 220-1779 | Zea mays |
| 611 | PHE0000356 | 272 | SVCT2-like permease | 34-1632 | Zea mays |
| 612 | PHE0000357 | 273 | maize tubby-like | 519-1958 | Zea mays |
| 613 | PHE0000358 | 274 | maize tubby-like | 517-1269 | Zea mays |
| 614 | PHE0000359 | 275 | soy HMG CoA synthase | 80-1441 | Glycine max |
| 615 | PHE0000360 | 276 | yeast HMGS-X96617 | 220-1695 | Saccharomyces |
| | | | | | cerevisiae |
| 616 | PHE0000361 | 277 | PAT1-like scarecrow 9 | 191-1900 | Zea mays |
| 617 | PHE0000362 | 278 | CDC28-related protein | 198-1484 | Zea mays |
| | | | kinase | | |
| 618 | PHE0000385 | 279 | H+ transporting ATPase | 176-2836 | Zea mays |
| 619 | PHE0000386 | 280 | cation-transporting | 222-2168 | Zea mays |
| | | | ATPase | | |
| 620 | PHE0000387 | 281 | yeast DRS2 (ALA1-like)- | 170-4237 | Saccharomyces |
| | | | L01795 | | cerevisiae |
| 621 | PHE0000388 | 282 | S. pombe ALA1-like- | 56-3832 | Schizosaccharomyces |
| | | | CAA21897 | | pombe |
| 622 | PHE0000389 | 283 | rice ALA1-like 1- | 47-1538, 1619-1925, 3116- | Oryza sativa |
| | | | BAA89544 | 3824, 3920-4043, 4143- | |
| | | | | 4362, 4590-5048, 5937-6153 | |
| 623 | PHE0000390 | 284 | rice chloroplastic | 136-1311 | Oryza sativa |
| | | | sedoheptulose-1,7- | | |
| | | | bisphosphatase- | | |
| 624 | PHE0000391 | 285 | rice cytosolic fructose- | 171-1187 | Oryza sativa |
| | | | 1,6-bisphosphatase | | |
| 625 | PHE0000392 | 286 | Wheat sedoheptulose-1,7- | 14-1192 | Triticum aestivum |
| | | | bisphosphatase | | |
| 626 | PHE0000394 | 287 | sedoheptulose-1,7- | 90-1238 | Chlorella |
| | | | bisphosphatase | | sorokiniana |
| 627 | PHE0000395 | 288 | soy phantastica | 275-1345 | Glycine max |
| 628 | PHE0000396 | 289 | soy phantastica 2 | 178-1260 | Glycine max |
| 629 | PHE0000397 | 290 | maize rough sheath 1 | 92-1144 | Zea mays |
| 630 | PHE0000398 | 291 | soy lg3-like 1 | 103-1026 | Glycine max |
| 631 | PHE0000399 | 292 | soy rough sheath1-like 1 | 144-1076 | Glycine max |
| 632 | PHE0000400 | 293 | soy G559-like | 301-1560 | Glycine max |
| 633 | PHE0000401 | 294 | soy G1635-like 1 | 28-888 | Glycine max |
| 634 | PHE0000402 | 295 | rice amino acid | 89-1426 | Oryza sativa |
| | | | transporter-like protein | | |
| 635 | PHE0000403 | 296 | corn amino acid permease | 116-1453 | Zea mays |
| 636 | PHE0000404 | 297 | rice proline transport | 313-1731 | Oryza sativa |
| | | | protein | | |
| 637 | PHE0000412 | 298 | corn monosaccharide | 75-1643 | Zea mays |
| | | | transporter 1 | | |
| 638 | PHE0000413 | 299 | soy monosaccharide | 132-1685 | Glycine max |
| | | | transporter 3 | | |
| 639 | PHE0000414 | 300 | corn monosaccharide | 141-1670 | Zea mays |
| | | | transporter 3 | | |
| 640 | PHE0000415 | 301 | soy monosaccharide | 160-1899 | Glycine max |
| | | | transporter 1 | | |
| 641 | PHE0000416 | 302 | corn monosaccharide | 74-1690 | Zea mays |
| | | | transporter 6 | | |
| 642 | PHE0000418 | 303 | corn monosaccharide | 146-1744 | Zea mays |
| | | | transporter 4 | | |
| 643 | PHE0000419 | 304 | soy monosaccharide | 63-1505 | Glycine max |
| | | | transporter 2 | | |
| 644 | PHE0000420 | 305 | soy sucrose transporter | 63-1595 | Glycine max |
| 645 | PHE0000421 | 306 | corn sucrose transporter 2 | 76-1599 | Zea mays |
| 646 | PHE0000422 | 307 | corn monosaccharide | 201-1763 | Zea mays |
| | | | transporter 8 | | |
| 647 | PHE0000423 | 308 | corn monosaccharide | 93-1634 | Zea mays |
| | | | transporter 7 | | |
| 648 | PHE0000425 | 309 | soy isoflavone synthase | 45-1607 | Glycine max |
| 649 | PHE0000426 | 310 | soy tgg1-like 2 | 52-1059 | Glycine max |
| 650 | PHE0000427 | 311 | GATE5-corn | 227-3139 | Zea mays |
| | | | SPA 1-like 1 | | |
| 651 | PHE0000428 | 312 | corn P1F3-like | 173-856 | Zea mays |
| 652 | PHE0000429 | 313 | soy Athb-2-like I | 78-932 | Glycine max |
| 653 | PHE0000430 | 314 | corn SUB1-like 1 | 44-1954 | Zea mays |
| 654 | PHE0000431 | 315 | soy GH3 protein | 42-1820 | Glycine max |
| 655 | PHE0000432 | 316 | corn 12- | 128-1240 | Zea mays |
| | | | oxophytodienoate | | |
| | | | reductase 1 | | |
| 656 | PHE0000433 | 317 | corn 12-oxo- | 166-1242 | Zea mays |
| | | | phytodienoate reductase- | | |
| | | | like 3 | | |
| 657 | PHE0000434 | 318 | corn 12- | 92-1210 | Zea mays |
| | | | oxophytodienoate | | |
| | | | reductase-like 4 | | |
| 658 | PH60000435 | 319 | corn hydroperoxide lyase | 83-1594 | Zea mays |
| 659 | PHE0000436 | 320 | rice cns1-like | 121-1242 | Oryza sativa |
| 660 | PHE0000437 | 321 | corn HCH1-like 1 | 42-1100 | Zea mays |
| 661 | PHE0000438 | 322 | corn HOP-like 1 | 88-1830 | Zea mays |
| 662 | PHE0000439 | 323 | corn HOP-like 2 | 65-1261 | Zea mays |
| 663 | PHE0000440 | 324 | rice CHIP-like 1 | 121-939 | Oryza sativa |
| 664 | PHE0000441 | 325 | corn CHIP-like 2 | 115-939 | Zea mays |
| 665 | PH60000451 | 326 | wheat SVP-like 1 | 149-736 | Triticum aestivum |
| 666 | PHE0000452 | 327 | corn SVP-like 3 | 75-749 | Zea mays |
| 667 | PHE0000453 | 328 | corn SVP-like 5 | 304-774, 956-1219 | Zea mays |
| 668 | PHE0000454 | 329 | fC-zmhuL1B3062-044- | 113-853 | Zea mays |
| | | | Q1-K1-B8 | | |
| 669 | PHE0000455 | 330 | corn E4/E8 binding | 253-2259 | Zea mays |
| | | | protein-like | | |
| 670 | PHE0000469 | 331 | yeast YKL091c- | 110-1042 | Saccharomyces |
| | | | Z28091 | | cerevisiae |
| 671 | PHE0000470 | 332 | corn Ssh1-like protein 1 | 57-1037 | Zea mays |
| 672 | PHE0000471 | 333 | corn Ssh1-like protein 3 | 89-841 | Zea mays |
| 673 | PHE0000472 | 334 | corn Ssh1-like protein 4 | 309-1196 | Zea mays |
| 674 | PHE0000473 | 335 | soy Ssh1-like protein 2 | 209-976 | Glycine max |
| | | | [ssh2] | | |
| 675 | PHE0000484 | 336 | soy JMT-like protein 1 | 26-1135 | Glycine max |
| 676 | PHE0000485 | 337 | corn JMT-like protein 1 | 39-1184 | Zea mays |
| 677 | PHE0000486 | 338 | corn JMT-like protein 2 | 63-1208 | Zea mays |
| 678 | PHE0000017 | 339 | corn AAA-ATPase 1 | 184-2214 | Zea mays |

Selection Methods for Transgenic Plants with Enhanced Agronomic Trait

Within a population of transgenic plants regenerated from plant cells transformed with the recombinant DNA many plants that survive to fertile transgenic plants that produce seeds and progeny plants will not exhibit an enhanced agronomic trait. Selection from the population is necessary to identify one or more transgenic plant cells that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality. Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat and rice plants.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### EXAMPLES

### Example 1

### Plant Expression Constructs

This example illustrates the construction of plasmids for transferring recombinant DNA into plant cells which can be regenerated into transgenic plants of this invention

### A. Plant Expression Constructs for Corn Transformation

A GATEWAY™ Destination (Invitrogen Life Technologies, Carlsbad, Calif.) plant expression vector, pMON65154, is constructed for use in preparation of constructs comprising recombinant polynucleotides for corn transformation. The elements of the expression vector are summarized in Table 3 below. Generally, pMON65154 comprises a selectable marker expression cassette comprising a Cauliflower Mosaic Virus 35S promoter operably linked to a gene encoding neomycin phosphotransferase II (nptII). The 3′ region of the selectable marker expression cassette comprises the 3′ region of the Agrobacterium tumefaciense nopaline synthase gene (nos) followed 3′ by the 3′ region of the potato proteinase inhibitor II (pinII) gene. The plasmid pMON 65154 further comprises a plant expression cassette into which a gene of interest may be inserted using GATEWAY™ cloning methods. The GATEWAY™ cloning cassette is flanked 5′ by a rice actin 1 promoter, exon and intron and flanked 3′ by the 3′ region of the potato pinII gene. Using GATEWAY™ methods, the cloning cassette may be replaced with a gene of interest. The vector pMON65J 54, and derivatives thereof comprising a gene of interest, are particularly useful in methods of plant transformation via direct DNA delivery, such as microprojectile bombardment.

TABLE 3
Elements of Plasmid pMON65154

| FUNCTION | ELEMENT | REFERENCE |
|---|---|---|
| Plant gene of | Rice actin 1 promoter | U.S. Pat. No. 5,641,876 |
| interest expression | Rice actin 1 exon 1, | U.S. Pat. No. 5,641,876 |
| cassette | intron 1enhancer | |
| Gene of interest | AttR1 | GATEWAY ™ Cloning |
| insertion site | | Technology Instruction Manual |
| | CmR gene | GATEWAY ™ Cloning |
| | | Technology Instruction Manual |
| | ccdA, ccdB genes | GATEWAY ™ Cloning |
| | | Technology Instruction Manual |
| | attR2 | GATEWAY ™ Cloning |
| | | Technology Instruction Manual |
| Plant gene of | Potato pinII 3′ region | An et al. (1989) Plant Cell |
| interest expression | | 1: 115-122 |
| cassette | | |
| Plant selectable | CaM V 35S promoter | U.S. Pat. No. 5,858,742 |
| marker expression | nptII selectable marker | U.S. Pat. No. 5,858,742 |
| cassette | nos 3′ region | U.S. Pat. No. 5,858,742 |
| | PinII 3′ region | An et al. (1989) Plant Cell |
| | | 1: 115-122 |
| Maintenance in | ColE1 origin of | |
| E. coli | replication F1 | |
| | origin of replication Bla | |
| | ampicillin resistance | |

A similar plasmid vector, pMON72472, is constructed for use in Agrobacterium mediated methods of plant transformation. pMON72472 comprises the gene of interest plant expression cassette, GATEWAY™ cloning, and plant selectable marker expression cassettes present in pMON65154. In addition, left and right T-DNA border sequences from Agrobacterium are added to the plasmid (Zambryski et al. (1982)). The right border sequence is located 5′ to the rice actin 1 promoter and the left border sequence is located 3′ to the pinII 3′ sequence situated 3′ to the nptII gene. Furthermore, pMON72472 comprises a plasmid backbone to facilitate replication of the plasmid in both E. coli and Agrobacterium tumefaciens. The backbone has an oriV wide host range origin of DNA replication functional in Agrobacterium, a pBR322 origin of replication functional in E. coli, and a spectinomycin/streptomycin resistance gene for selection in both E. coli and Agrobacterium.

Vectors similar to those described above may be constructed for use in Agrobacterium or microprojectile bombardment maize transformation systems where the rice actin 1 promoter in the plant expression cassette portion is replaced with other desirable promoters including, but not limited to a corn globulin 1 promoter, a maize oleosin promoter, a glutelin 1 promoter, an aldoluse promoter, a zein Z27 promoter, a pyruvate orthophosphate dikinase (PPDK) promoter, a a soybean 7S alpha promoter, a peroxiredoxin antioxidant (Per1) promoter and a CaMV 35S promoter. Protein coding segments are amplified by PCR prior to insertion into vectors such as described above. Primers for PCR amplification can be designed at or near the start and stop codons of the coding sequence, in order to eliminate most of the 5′ and 3′ untranslated regions. For GATEWAY cloning methods, PCR products are tailed with attB1 and autB2 sequences, purified then recombined into a destination vectors to produce an expression vector for use in transformation.

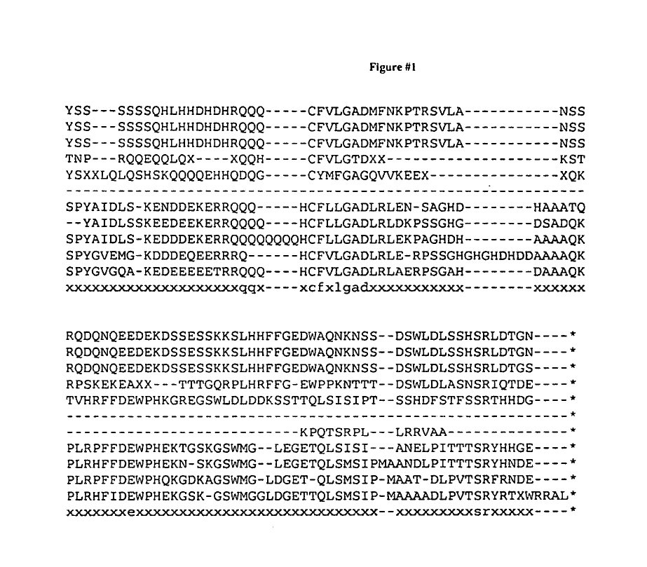

Another base corn plant transformation vector pMON93039, as set forth in SEQ ID NO: 24150, illustrated in Table 4 and FIG. 2, was fabricated for use in preparing recombinant DNA for Agrobacterium-mediated transformation into corn tissue.

TABLE 4

| | | | Coordinates of |
|---|---|---|---|
| | | | SEQ ID NO: |
| function | Name | Annotation | 24150 |
| Agrobacterium | B-AGRtu.right border | Agro right border sequence, | 11364-11720 |
| T-DNA transfer | | essential for transfer of T-DNA. | |
| Gene of interest | E-Os.Act1 | upstream promoter region of | 19-775 |
| expression | | the rice actin 1 gene | |
| cassette | E-CaMV.35S.2xA1-B3 | duplicated35S A1-B3 | 788-1120 |
| | | domain without TATA box | |
| | P-Os.Act1 | promoter region of the rice | 1125-1204 |
| | | actin 1 gene | |
| | L-Ta.Lhcb1 | 5′ untranslated leader of | 1210-1270 |
| | | wheat major chlorophyll a/b | |
| | | binding protein | |
| | I-Os.Act1 | first intron and flanking | 1287-1766 |
| | | UTR exon sequences from | |
| | | the rice actin 1 gene | |
| | T-St.Pis4 | 3′ non-translated region of | 1838-2780 |
| | | the potato proteinase | |
| | | inhibitor II gene which | |
| | | functions to direct | |
| | | polyadenylation of the | |
| | | mRNA | |
| Plant selectable | P-Os.Act1 | Promoter from the rice actin | 2830-3670 |
| marker | | 1 gene | |
| expression | L-Os.Act1 | first exon of the rice actin 1 | 3671-3750 |
| cassette | | gene | |
| | I-Os.Act 1 | first intron and flanking | 3751-4228 |
| | | UTR exon sequences from | |
| | | the rice actin 1 gene | |
| | TS-At.ShkG-CTP2 | Transit peptide region of | 4238-4465 |
| | | Arabidopsis EPSPS | |
| | CR-AGRtu.aroA- | Coding region for bacterial | 4466-5833 |
| | CP4.nat | strain CP4 native aroA gene. | |
| | T-AGRtu.nos | A 3′ non-translated region of | 5849-6101 |
| | | the nopaline synthase gene | |
| | | of Agrobacterium | |
| | | tumefaciens Ti plasmid | |
| | | which functions to direct | |
| | | polyadenylation of the | |
| | | mRNA. | |
| Agrobacterium | B-AGRtu.left border | Agro left border sequence, | 6168-6609 |
| T-DNA transfer | | essential for transfer of T- | |
| | | DNA. | |
| Maintenance in | OR-Ec.oriV-RK2 | The vegetative origin of | 6696-7092 |
| E. coli | | replication from plasmid | |
| | | RK2. | |
| | CR-Ec.rop | Coding region for repressor | 8601-8792 |
| | | of primer from the ColE1 | |
| | | plasmid. Expression of this | |
| | | gene product interferes with | |
| | | primer binding at the origin | |
| | | of replication, keeping | |
| | | plasmid copy number low. | |
| | OR-Ec.ori-ColE1 | The minimal origin of | 9220-9808 |
| | | replication from the E. coli | |
| | | plasmid ColE1. | |
| | P-Ec.aadA-SPC/STR | remoter for Tn7 | 10339-10380 |
| | | adenylyltransferase | |
| | | (AAD(3′′)) | |
| | CR-Ec.aadA-SPC/STR | Coding region for Tn7 | 10381-11169 |
| | | adenylyltransferase | |
| | | (AAD(3′′)) conferring | |
| | | spectinomycin and | |
| | | streptomycin resistance. | |
| | T-Ec.aadA-SPC/STR | 3′ UTR from the Tn7 | 11170-11227 |
| | | adenylyltransferase | |
| | | (AAD(3′′)) gene of E. coli. | |

B. Plant Expression Constructs for Soy and Canola Transformation

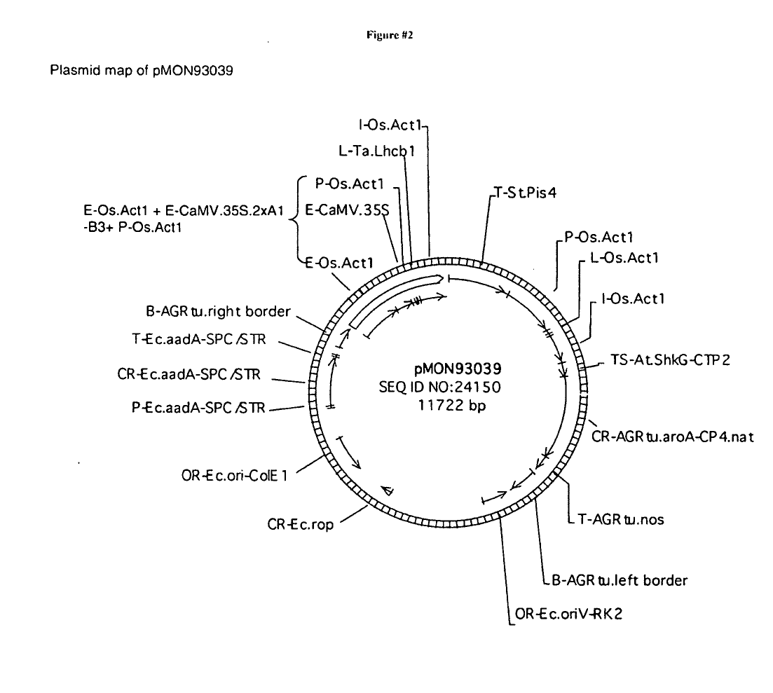

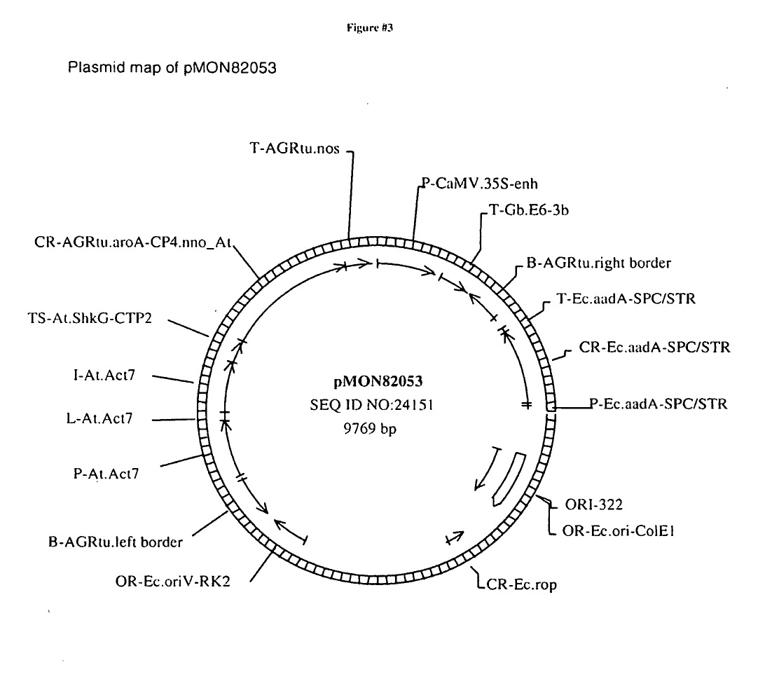

Plasmids for use in transformation of soybean and canola were also prepared. Elements of an exemplary common expression vector pMON82053 are shown in Table 5 below and FIG. 3.

TABLE 5

| | | | Coordinates of |
|---|---|---|---|
| | | | SEQ ID NO: |
| Function | Name | Annotation | 24151 |
| Agrobacterium T- | B-AGRtu.left border | Agro left border sequence, essential for | 6144-6585 |
| DNA transfer | | transfer of T-DNA. | |
| Plant selectable | P-At.Act7 | Promoter from the Arabidopsis actin 7 gene | 6624-7861 |
| marker expression | L-At.Act7 | 5′UTR of Arabidopsis Act7 gene | |
| cassette | I-At.Act7 | Intron from the Arabidopsis actin7 gene | |
| | TS-At.ShkG-CTP2 | Transit peptide region of Arabidopsis | 7864-8091 |
| | | EPSPS | |
| | CR-AGRtu.aroA- | Synthetic CP4 coding region with dicot | 8092-9459 |
| | CP4.nno_At | preferred codon usage. | |
| | T-AGRtu.nos | A 3′ non-translated region of the nopaline | 9466-9718 |
| | | synthase gene of Agrobacterium | |
| | | tumefaciens Ti plasmid which functions to | |
| | | direct polyadenylation of the mRNA. | |
| Gene of interest | P-CaMV.35S-enh | Promoter for 35S RNA from CaMV | 1-613 |
| expression cassette | | containing a duplication of the −90 to −350 | |
| | | region. | |
| | T-Gb.E6-3h | 3′ untranslated region from the fiber protein | 688-1002 |
| | | E6 gene of sea-island cotton. | |
| Agrobacterium T- | B-AGRtu.right | Agro right border sequence, essential for | 1033-1389 |
| DNA transfer | border | transfer of T-DNA. | |
| Maintenance in E. | OR-Ec.oriV-RK2 | The vegetative origin of replication from | 5661-6057 |
| coli | | plasmid RK2. | |
| | CR-Ec.rop | Coding region for repressor of primer from | 3961-4152 |
| | | the ColE1 plasmid. Expression of this gene | |
| | | product interferes with primer binding at the | |
| | | origin of replication, keeping plasmid copy | |
| | | number low. | |
| | OR-Ec.ori-ColE1 | The minimal origin of replication from the | 2945-3533 |
| | | E. coli plasmid ColE1. | |
| | P-Ec.aadA-SPC/STR | Promoter for Tn7 adenylyltransferase | 2373-2414 |
| | | (AAD(3′′)) | |
| | CR-Ec.aadA- | Coding region for Tn7 adenylyltransferase | 1584-2372 |
| | SPC/STR | (AAD(3′′)) conferring spectinomycin and | |
| | | streptomycin resistance. | |
| | T-Ec.aadA-SPC/STR | 3′ UTR from the Tn7 adenylyltransferase | 1526-1583 |
| | | (AAD(3′′)) gene of E. coli. | |

Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 2 is amplified by PCR prior to insertion into the insertion site within the gene of interest expression cassette of one of the base vectors.

Vectors similar to that described above may be constructed for use in Agrobacterium mediated soybean transformation systems where the enhanced 35S promoter in the plant expression cassette portion is replaced with other desirable promoters including, but not limited to a napin promoter and an Arabidopsis SSU promoter. Protein coding segments are amplified by PCR prior to insertion into vectors such as described above. Primers for PCR amplification can be designed at or near the start and stop codons of the coding sequence, in order to eliminate most of the 5′ and 3′ untranslated regions.

C. Cotton Transformation Vector

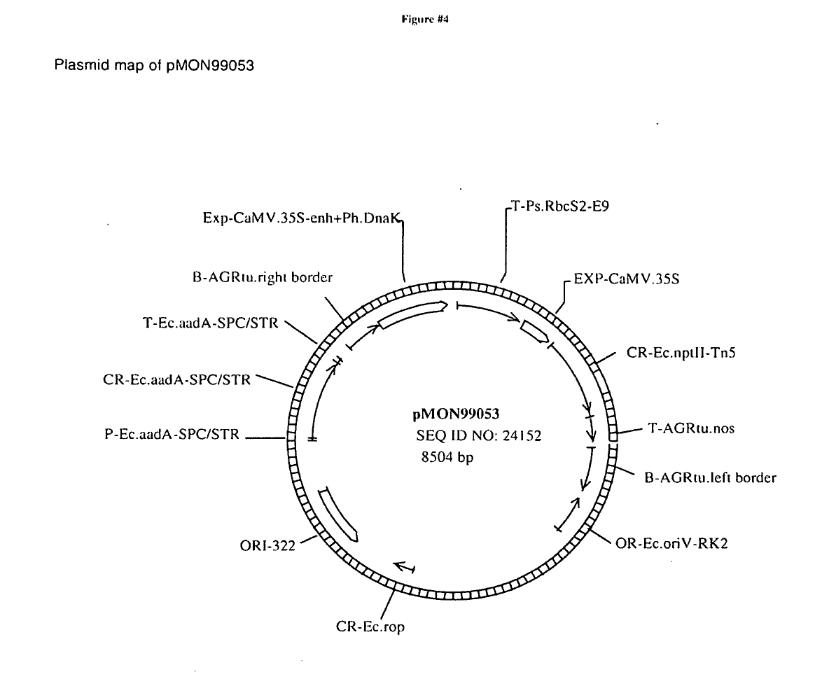

Plasmids for use in transformation of cotton are also prepared. Elements of an exemplary common expression vector plasmid pMON99053 are shown in Table 6 below and FIG. 4. Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 2 is amplified by PCR prior to insertion into the insertion site within the gene of interest expression cassette of one of the base vectors.

TABLE 6

| | | | Coordinates of |
|---|---|---|---|
| | | | SEQ ID NO: |
| function | Name | annotation | 24152 |
| Agrobacterium | B-AGRtu.right border | Agro right border sequence, | 11364-11720 |
| T-DNA transfer | | essential for transfer of T-DNA. | |
| Gene of interest | Exp-CaMV.35S- | Enhanced version of the 35S | 7794-8497 |
| expression | enh + ph.DnaK | RNA promoter from CaMV plus | |
| cassette | | the petunia hsp70 5′ untranslated | |
| | | region | |
| | T-Ps.RbcS2-E9 | The 3′ non-translated region of | 67-699 |
| | | the pea RbcS2 gene which | |
| | | functions to direct | |
| | | polyadenylation of the mRNA. | |
| Plant selectable | Exp-CaMV.35S | Promoter from the rice actin 1 | 730-1053 |
| marker | | gene | |
| expression | CR-Ec.nptII-Tn5 | first exon of the rice actin 1 gene | 1087-1881 |
| cassette | | | |
| | T-AGRtu.nos | A 3′ non-translated region of the | 1913-2165 |
| | | nopaline synthase gene of | |
| | | Agrobacterium tumefaciens Ti | |
| | | plasmid which functions to | |
| | | direct polyadenylation of the | |
| | | mRNA. | |
| Agrobacterium | B-AGRtu.left border | Agro left border sequence, | 2211-2652 |
| T-DNA transfer | | essential for transfer of T-DNA. | |
| Maintenance in | OR-Ec.oriV-RK2 | The vegetative origin of | 2739-3135 |
| E. coli | | replication from plasmid RK2. | |
| | CR-Ec.rop | Coding region for repressor of | 4644-4835 |
| | | primer from the ColE1 plasmid. | |
| | | Expression of this gene product | |
| | | interferes with primer binding at | |
| | | the origin of replication, keeping | |
| | | plasmid copy number low. | |
| | OR-Ec.ori-ColE1 | The minimal origin of | 5263-5851 |
| | | replication from the E. coli | |
| | | plasmid ColE1. | |
| | P-Ec.aadA-SPC/STR | romoter for Tn7 | 6382-6423 |
| | | adenylyltransferase (AAD(3′′)) | |
| | CR-Ec.aadA-SPC/STR | Coding region for Tn7 | 6424-7212 |
| | | adenylyltransferase (AAD(3′′)) | |
| | | conferring spectinomycin and | |
| | | streptomycin resistance. | |
| | T-Ec.aadA-SPC/STR | 3′ UTR from the Tn7 | 7213-7270 |
| | | adenylyltransferase (AAD(3′′)) | |
| | | gene of E. coli. | |

### Example 2

### Corn Transformation

This example illustrates plant cell transformation methods useful in producing transgenic corn plant cells, plants, seeds and pollen of this invention and the production and identification of transgenic corn plants and seed with an enhanced trait, i.e. enhanced water use efficiency, enhanced cold tolerance, increased yield, enhanced nitrogen use efficiency, enhanced seed protein and enhanced seed oil. Plasmid vectors were prepared by cloning DNA identified in Table 1 in the identified base vectors for use in corn transformation of corn plant cells to produce transgenic corn plants and progeny plants, seed and pollen.

For Agrobacterium-mediated transformation of corn embryo cells corn plants of a readily transformable line (designated LH59) is grown in the greenhouse and ears harvested when the embryos are 1.5 to 2.0 mm in length. Ears are surface sterilized by spraying or soaking the ears in 80% ethanol, followed by air drying. Immature embryos are isolated from individual kernels on surface sterilized ears. Prior to inoculation of maize cells, Agrobacterium cells are grown overnight at room temperature. Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media, and paromomycin resistant callus identified as containing the recombinant DNA in an expression cassette.

For transformation by microprojectile bombardment immature maize embryos are isolated and cultured 3-4 days prior to bombardment. Prior to microprojectile bombardment, a suspension of gold particles is prepared onto which the desired recombinant DNA expression cassettes are precipitated. DNA is introduced into maize cells as described in U.S. Pat. Nos. 5,550,318 and 6,399,861 using the electric discharge particle acceleration gene delivery device. Following microprojectile bombardment, tissue is cultured in the dark at 27 degrees C. Additional transformation methods and materials for making transgenic plants of this invention, for example, various media and recipient target cells, transformation of immature embryos and subsequence regeneration of fertile transgenic plants are disclosed in U.S. Pat. Nos. 6,194,636 and 6,232,526 and U.S. patent application Ser. No. 09/757,089, which are incorporated herein by reference.

To regenerate transgenic corn plants a callus of transgenic plant cells resulting from transformation is placed on media to initiate shoot development in plantlets which are transferred to potting soil for initial growth in a growth chamber at 26 degrees C. followed by a mist bench before transplanting to 5 inch pots where plants are grown to maturity. The regenerated plants are self fertilized and seed is harvested for use in one or more methods to select seed, seedlings or progeny second generation transgenic plants (R2 plants) or hybrids, e.g. by selecting transgenic plants exhibiting an enhanced trait as compared to a control plant.

Transgenic corn plant cells are transformed with recombinant DNA from each of the genes identified in Table 2. Progeny transgenic plants and seed of the transformed plant cells are screened for enhanced water use efficiency, enhanced cold tolerance, increased yield, enhanced nitrogen use efficiency, enhanced seed protein and enhanced seed oil as reported in Example 5.

### Example 3

### Soybean Transformation

This example illustrates plant transformation useful in producing the transgenic soybean plants of this invention and the production and identification of transgenic seed for transgenic soybean having enhanced water use efficiency, enhanced cold tolerance, increased yield, enhanced nitrogen use efficiency, enhanced seed protein and enhanced seed oil.

For Agrobacterium mediated transformation, soybean seeds are germinated overnight and the meristem explants excised. The meristems and the explants are placed in a wounding vessel. Soybean explants and induced Agrobacterium cells from a strain containing plasmid DNA with the gene of interest cassette and a plant selectable marker cassette are mixed no later than 14 hours from the time of initiation of seed germination and wounded using sonication. Following wounding, explants are placed in co-culture for 2-5 days at which point they are transferred to selection media for 6-8 weeks to allow selection and growth of transgenic shoots. Trait positive shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil. Additionally, a DNA construct can be transferred into the genome of a soybean cell by particle bombardment and the cell regenerated into a fertile soybean plant as described in U.S. Pat. No. 5,015,580, herein incorporated by reference.

Transgenic soybean plant cells are transformed with recombinant DNA from each of the genes identified in Table 2. Progeny transgenic plants and seed of the transformed plant cells are screened for enhanced water use efficiency, enhanced cold tolerance, increased yield, enhanced nitrogen use efficiency, enhanced seed protein and enhanced seed oil as reported in Example 5.

### Example 4

### Cotton Transgenic Plants with Enhanced Agronomic Traits

Cotton transformation is performed as generally described in WO0036911 and in U.S. Pat. No. 5,846,797. Transgenic cotton plants containing each of the recombinant DNA having a sequence of SEQ ID NO: 1 through SEQ ID NO: 339 are obtained by transforming with recombinant DNA from each of the genes identified in Table 2. Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA. The specified culture conditions are growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

The transgenic cotton plants of this invention are identified from among the transgenic cotton plants by agronomic trait screening as having increased yield and enhanced water use efficiency.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterization are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant

Transgenic canola plant cells are transformed with recombinant DNA from each of the genes identified in Table 2. Transgenic progeny plants and seed of the transformed plant cells are screened for enhanced water use efficiency, enhanced cold tolerance, increased yield, enhanced nitrogen use efficiency, enhanced seed protein and enhanced seed oil as reported in Example 7.

### Example 6

### Homolog Identification

This example illustrates the identification of homologs of proteins encoded by the DNA identified in Table 2 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 340 through SEQ ID NO: 678 using NCBI “blastp” program with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 340 through SEQ ID NO: 678 using NCBI “blastp” program with E-value cutoff of 1e-4. Up to 1000 top hits were kept. A BLAST searchable database was constructed based on these hits, and is referred to as “SubDB”. SubDB was queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs from a large number of distinct organisms were identified and are reported by amino acid sequences of SEQ ID NO: 679 through SEQ ID NO: 24149. These relationship of proteins of SEQ ID NO: 340 through 678 and homologs of SEQ ID NO: 679 through 24149 is identified in Table 7. The source organism for each homolog is found in the Sequence Listing.

### Example 7

### Selection of Transgenic Plants with Enhanced Agronomic Trait(s)

This example illustrates identification of plant cells of the invention by screening derived plants and seeds for enhanced trait. Transgenic corn seed and plants with recombinant DNA identified in Table 2 are prepared by plant cells transformed with DNA that is stably integrated into the genome of the corn cell. Transgenic corn plant cells are transformed with recombinant DNA from each of the genes identified in Table 2. Progeny transgenic plants and seed of the transformed plant cells are screened for enhanced water use efficiency, enhanced cold tolerance, increased yield, enhanced nitrogen use efficiency, enhanced seed protein and enhanced seed oil as compared to control plants.

### A. Selection for Enhanced Nitrogen Use Efficiency

The physiological efficacy of transgenic corn plants (tested as hybrids) can be tested for nitrogen use efficiency (NUE) traits in a high-throughput nitrogen (N) selection method. The collected data are compared to the measurements from wildtype controls using a statistical model to determine if the changes are due to the transgene. Raw data were analyzed by SAS software. Results shown herein are the comparison of transgenic plants relative to the wildtype controls.

### (1) Media Preparation for Planting a NUE Protocol

Planting materials used: Metro Mix 200 (vendor: Hummert) Cat. #10-0325, Scotts Micro Max Nutrients (vendor: Hummert) Cat. #07-6330, OS 4⅓″×3⅞″ pots (vendor: Hummert) Cat. #16-1415. OS trays (vendor: Hummert) Cat. #16-1515, Hoagland's macronutrients solution, Plastic 5″ stakes (vendor Hummert) yellow Cat. #49-1569, white Cat. #49-1505, Labels with numbers indicating material contained in pots. Fill 500 pots to rim with Metro Mix 200 to a weight of ˜140 g/pot. Pots are filled uniformly by using a balancer. Add 0.4 g of Micro Max nutrients to each pot. Stir ingredients with spatula to a depth of 3 inches while preventing material loss.

### (2) Planting a NUE Selection in the Greenhouse

(a) Seed Germination—Each pot is lightly altered twice using reverse osmosis purified water. The first watering is scheduled to occur just before planting; and the second watering, after the seed has been planted in the pot. Ten Seeds of each entry (1 seed per pot) are planted to select eight healthy uniform seedlings. Additional wild type controls are planted for use as border rows. Alternatively, 15 seeds of each entry (I seed per pot) are planted to select 12 healthy uniform seedlings (this larger number of plantings is used for the second, or confirmation, planting). Place pots on each of the 12 shelves in the Conviron growth chamber for seven days. This is done to allow more uniform germination and early seedling growth. The following growth chamber settings are 25° C./day and 22° C./night, 14 hours light and ten hours dark, humidity ˜80%. and light intensity ˜350 μmol/m²/s (at pot level). Watering is done via capillary matting similar to greenhouse benches with duration of ten minutes three times a day.

(b) Seedling transfer—After seven days, the best eight or 12 seedlings for the first or confirmation pass runs, respectively, are chosen and transferred to greenhouse benches. The pots are spaced eight inches apart (center to center) and are positioned on the benches using the spacing patterns printed on the capillary matting. The Vattex matting creates a 384-position grid, randomizing all range, row combinations. Additional pots of controls are placed along the outside of the experimental block to reduce border effects.

Plants are allowed to grow for 28 days under the low N run or for 23 days under the high N run. The macronutrients are dispensed in the form of a macronutrient solution (see composition below) containing precise amounts of N added (2 mM NH₄NO₃ for limiting N selection and 20 mM NH₄NO₃ for high N selection runs). Each pot is manually dispensed 100 ml of nutrient solution three times a week on alternate days starting at eight and ten days after planting for high N and low N runs, respectively. On the day of nutrient application, two 20 min waterings at 05:00 and 13:00 are skipped. The vattex matting should be changed every third run to avoid N accumulation and buildup of root matter. Table 8 shows the amount of nutrients in the nutrient solution for either the low or high nitrogen selection.

TABLE 8

| | 2 mM NH₄NO₃ | 20 mM NH₄NO₃ |
|---|---|---|
| | (Low Nitrogen | (high Nitrogen |
| | Growth Condition, | Growth Condition, |
| Nutrient Stock | Low N) mL/L | High N) mL/L |
| 1M NH₄NO₃ | 2 | 20 |
| 1M KH₂PO₄ | 0.5 | 0.5 |
| 1M MgSO₄•7H₂O | 2 | 2 |
| 1M CaCl₂ | 2.5 | 2.5 |
| 1M K₂SO₄ | 1 | 1 |

Note:
Adjust pH to 5.6 with HCl or KOH

(c) Harvest Measurements and Data Collection—After 28 days of plant growth for low N runs and 23 days of plant growth for high N runs, the following measurements are taken (phenocodes in parentheses): total shoot fresh mass (g) (SFM) measured by Sartorius electronic balance, V6 leaf chlorophyll measured by Minolta SPAD meter (relative units) (LC), V6 leaf area (cm²) (LA) measured by a Li-Cor leaf area meter, V6 leaf fresh mass (g) (LFM) measured by Sartorius electronic balance, and V6 leaf dry mass (g) (LDM) measured by Sartorius electronic balance. Raw data were analyzed by SAS software. Results shown are the comparison of transgenic plants relative to the wildtype controls.

To take a leaf reading, samples were excised from the V6 leaf. Since chlorophyll meter readings of corn leaves are affected by the part of the leaf and the position of the leaf on the plant that is sampled, SPAD meter readings were done on leaf six of the plants. Three measurements per leaf were taken, of which the first reading was taken from a point one-half the distance between the leaf tip and the collar and halfway from the leaf margin to the midrib while two were taken toward the leaf tip. The measurements were restricted in the area from ½ to ¾ of the total length of the leaf (from the base) with approximately equal spacing between them. The average of the three measurements was taken from the SPAD machine.

Leaf fresh mass is recorded for an excised V6 leaf, the leaf is placed into a paper bag. The paper bags containing the leaves are then placed into a forced air oven at 80° C. for 3 days. After 3 days, the paper bags are removed from the oven and the leaf dry mass measurements are taken.

From the collected data, two derived measurements are made: (1)Leaf chlorophyll area (LCA), which is a product of V6 relative chlorophyll content and its leaf area (relative units).

Leaf chlorophyll area=leaf chlorophyll X leaf area. This parameter gives an indication of the spread of chlorophyll over the entire leaf area: (2)specific leaf area (LSA) is calculated as the ratio of V6 leaf area to its dry mass (cm²/g dry mass), a parameter also recognized as a measure of NUE.

A list of recombinant DNA constructs which improved growth in high nitrogen in transgenic plants is illustrated in Table 9.

TABLE 9

| | | | | Positive | Confirmed |
|---|---|---|---|---|---|
| | | | | events/ | events/ |
| NUC | PEP | | | Total | Actual events |
| SEQ | SEQ | | | events | with confirmation |
| ID | ID | PHE ID | Construct | screened | attempted |
| 8 | 347 | PHE0000012 | PMON67808 | 1/5 | 0/0 |
| 12 | 351 | PHE0000016 | PMON67750 | 1/3 | 0/0 |
| 16 | 355 | PHE0000022 | PMON67826 | 1/1 | 0/0 |
| 16 | 355 | PHE0000022 | PMON67826 | 1/3 | 0/0 |
| 33 | 372 | PHE0000039 | PMON67807 | 1/2 | 0/0 |
| 34 | 373 | PHE0000040 | PMON77889 | 1/4 | 0/0 |
| 46 | 385 | PHE0000051 | PMON68859 | 1/2 | 0/0 |
| 47 | 386 | PHE0000052 | PMON67813 | 2/2 | 0/0 |
| 54 | 393 | PHE0000058 | PMON68351 | 1/2 | 0/0 |
| 62 | 401 | PHE0000067 | PMON67816 | 4/4 | 3/4 |
| 64 | 403 | PHE0000069 | PMON67821 | 1/1 | 0/0 |
| 68 | 407 | PHE0000073 | PMON68357 | 3/3 | 0/0 |
| 72 | 411 | PHE0000077 | PMON67827 | 3/4 | 1/4 |
| 101 | 440 | PHE0000116 | PMON68367 | 2/2 | 0/0 |
| 105 | 444 | PHE0000120 | PMON68853 | 2/2 | 0/0 |
| 108 | 447 | PHE0000123 | PMON68855 | 2/3 | 0/2 |
| 112 | 451 | PHE0000127 | PMON68887 | 1/1 | 0/0 |
| 116 | 455 | PHE0000153 | PMON67817 | 4/5 | 4/5 |
| 117 | 456 | PHE0000154 | PMON67818 | 1/2 | 0/2 |
| 120 | 459 | PHE0000158 | PMON73169 | 2/2 | 0/2 |
| 135 | 474 | PHE0000177 | PMON68881 | 1/2 | 1/2 |
| 136 | 475 | PHE0000178 | PMON73166 | 1/2 | 0/0 |
| 143 | 482 | PHE0000185 | PMON69468 | 1/3 | 0/0 |
| 146 | 485 | PHE0000188 | PMON73167 | 2/2 | 0/0 |
| 169 | 508 | PHE0000235 | PMON73161 | 1/2 | 0/0 |
| 176 | 515 | PHE0000243 | PMON72467 | 2/2 | 0/2 |
| 190 | 529 | PHE0000264 | PMON68866 | 3/3 | 0/0 |
| 193 | 532 | PHE0000267 | PMON68867 | 2/2 | 1/2 |
| 204 | 543 | PHE0000279 | PMON68896 | 3/3 | 2/2 |
| 214 | 553 | PHE0000291 | PMON72455 | 3/3 | 1/2 |
| 234 | 573 | PHE0000312 | PMON72456 | 1/3 | 0/2 |
| 235 | 574 | PHE0000313 | PMON68378 | 1/2 | 1/2 |
| 236 | 575 | PHE0000314 | PMON68379 | 4/4 | 1/4 |
| 237 | 576 | PHE0000315 | PMON68381 | 2/4 | 0/2 |
| 239 | 578 | PHE0000317 | PMON68380 | 2/2 | 0/0 |
| 249 | 588 | PHE0000330 | PMON73164 | 2/3 | 0/0 |
| 264 | 603 | PHE0000347 | PMON68386 | 1/2 | 0/0 |
| 265 | 604 | PHE0000349 | PMON68389 | 1/1 | 0/0 |
| 266 | 605 | PHE0000350 | PMON74410 | 1/2 | 1/2 |
| 268 | 607 | PHE0000352 | PMON74409 | 1/5 | 0/5 |
| 269 | 608 | PHE0000353 | PMON73160 | 2/2 | 0/0 |
| 284 | 623 | PHE0000390 | PMON67836 | 1/2 | 0/0 |
| 296 | 635 | PHE0000403 | PMON67831 | 1/2 | 0/0 |
| 301 | 640 | PHE0000415 | PMON67846 | 4/5 | 0/5 |
| 303 | 642 | PHE0000418 | PMON69497 | 2/4 | 1/4 |
| 304 | 643 | PHE0000419 | PMON67848 | 1/2 | 0/2 |
| 324 | 663 | PHE0000440 | PMON72473 | 3/5 | 0/0 |
| 331 | 670 | PHE0000469 | PMON68636 | 1/3 | 0/0 |

A list of recombinant DNA constructs which improved growth in limited nitrogen in transgenic plants is illustrated in Table 10.

TABLE 10

| | | | | | Confirmed |
|---|---|---|---|---|---|
| | | | | Positive | events/ |
| | | | | events/ | Actual |
| NUC | PEP | | | Total | events with |
| SEQ | SEG | PHE | | events | confirmation |
| ID | ID | ID | Construct | screened | attempted |
| 2 | 341 | PHE0000006 | PMON68861 | 1/5 | 0/1 |
| 5 | 344 | PHE0000010 | PMON67800 | 4/5 | 2/4 |
| 8 | 347 | PHE0000012 | PMON67806 | 1/3 | 1/1 |
| 16 | 355 | PHE0000022 | PMON67826 | 3/3 | 1/3 |
| 17 | 356 | PHE0000024 | PMON68354 | 1/4 | 0/4 |
| 20 | 359 | PHE0000227 | PMON68376 | 2/4 | 0/0 |
| 24 | 363 | PHE0000027 | PMON85009 | 2/6 | 0/0 |
| 31 | 370 | PHE0000034 | PMON67805 | 2/6 | 0/2 |
| 32 | 371 | PHE0000038 | PMON68383 | 1/6 | 0/2 |
| 33 | 372 | PHE0000039 | PMON67807 | 1/3 | 0/2 |
| 34 | 373 | PHE0000040 | PMON67801 | 1/5 | 0/0 |
| 34 | 373 | PHE0000040 | PMON77889 | 4/4 | 4/4 |
| 34 | 373 | PHE0000040 | PMON92405 | 1/6 | 0/0 |
| 37 | 376 | PHE0000045 | PMON81293 | 2/8 | 0/0 |
| 40 | 379 | PHE0000244 | PMON68372 | 2/2 | 1/2 |
| 41 | 380 | PHE0000245 | PMON68373 | 3/4 | 1/4 |
| 41 | 380 | PHE0000245 | PMON84737 | 1/7 | 0/6 |
| 42 | 381 | PHE0000246 | PMON68374 | 2/3 | 0/0 |
| 43 | 382 | PHE0000247 | PMON68375 | 1/3 | 0/0 |
| 44 | 383 | PHE0000106 | PMON69457 | 1/1 | 0/0 |
| 44 | 383 | PHE0000106 | PMON92483 | 3/6 | 0/1 |
| 46 | 385 | PHE0000051 | PMON68859 | 2/2 | 1/2 |
| 47 | 386 | PHE0000052 | PMON67813 | 1/4 | 0/2 |
| 51 | 390 | PHE0000055 | PMON68355 | 1/3 | 0/2 |
| 53 | 392 | PHE0000057 | PMON68350 | 1/4 | 1/4 |
| 54 | 393 | PHE0000058 | PMON68351 | 1/4 | 0/3 |
| 56 | 395 | PHE0000060 | PMON68356 | 1/3 | 0/2 |
| 59 | 398 | PHE0000064 | PMON67804 | 1/6 | 0/0 |
| 61 | 400 | PHE0000292 | PMON68888 | 1/2 | 0/0 |
| 62 | 401 | PHE0000067 | PMON67816 | 4/4 | 2/4 |
| 62 | 401 | PHE0000067 | PMON92814 | 1/6 | 0/0 |
| 63 | 402 | PHE0000068 | PMON67824 | 1/2 | 0/0 |
| 64 | 403 | PHE0000069 | PMON67821 | 4/5 | 2/3 |
| 65 | 404 | PHE0000070 | PMON67825 | 1/3 | 0/0 |
| 67 | 406 | PHE0000072 | PMON67828 | 1/2 | 0/0 |
| 72 | 411 | PHE0000077 | PMON67827 | 2/6 | 0/2 |
| 72 | 411 | PHE0000077 | PMON77890 | 1/2 | 0/0 |
| 74 | 413 | PHE0000079 | PMON67752 | 2/5 | 0/0 |
| 79 | 418 | PHE0000086 | PMON67812 | 1/4 | 0/0 |
| 80 | 419 | PHE0000089 | PMON84111 | 2/4 | 0/0 |
| 99 | 438 | PHE0000114 | PMON68361 | 1/2 | 0/0 |
| 100 | 439 | PHE0000115 | PMON68362 | 1/1 | 0/0 |
| 101 | 440 | PHE0000116 | PMON68367 | 1/7 | 0/2 |
| 102 | 441 | PHE0000117 | PMON68368 | 1/2 | 0/2 |
| 103 | 442 | PHE0000118 | PMON67811 | 6/7 | 2/6 |
| 104 | 443 | PHE0000119 | PMON68363 | 1/4 | 0/1 |
| 105 | 444 | PHE0000120 | PMON68853 | 2/6 | 0/5 |
| 108 | 447 | PHE0000123 | PMON68855 | 3/4 | 0/3 |
| 110 | 449 | PHE0000125 | PMON68369 | 3/7 | 0/4 |
| 111 | 450 | PHE0000126 | PMON69458 | 4/7 | 1/4 |
| 112 | 451 | PHE0000127 | PMON68887 | 2/5 | 0/0 |
| 114 | 453 | PHE0000133 | PMON68860 | 1/4 | 0/0 |
| 116 | 455 | PHE0000153 | PMON67817 | 1/6 | 0/5 |
| 117 | 456 | PHE0000154 | PMON67818 | 2/2 | 1/2 |
| 120 | 459 | PHE0000158 | PMON73169 | 2/2 | 2/2 |
| 129 | 468 | PHE0000168 | PMON68857 | 1/5 | 0/5 |
| 135 | 474 | PHE0000177 | PMON68881 | 2/3 | 2/3 |
| 135 | 474 | PHE0000177 | PMON92800 | 4/6 | 0/0 |
| 138 | 477 | PHE0000180 | PMON83753 | 1/7 | 0/0 |
| 140 | 479 | PHE0000182 | PMON74420 | 3/3 | 1/2 |
| 141 | 480 | PHE0000183 | PMON80258 | 2/5 | 0/5 |
| 142 | 481 | PHE0000184 | PMON84985 | 2/5 | 0/0 |
| 143 | 482 | PHE0000185 | PMON69468 | 3/4 | 1/4 |
| 146 | 485 | PHE0000188 | PMON73167 | 1/4 | 0/2 |
| 151 | 490 | PHE0000219 | PMON68865 | 1/2 | 0/0 |
| 169 | 508 | PHE0000235 | PMON73161 | 1/2 | 1/2 |
| 176 | 515 | PHE0000243 | PMON72467 | 1/2 | 0/2 |
| 182 | 521 | PHE0000254 | PMON73172 | 1/4 | 0/0 |
| 183 | 522 | PHE0000255 | PMON72459 | 1/1 | 1/1 |
| 190 | 529 | PHE0000264 | PMON68866 | 1/4 | 0/3 |
| 191 | 531 | PHE0000266 | PMON69470 | 3/3 | 1/3 |
| 193 | 532 | PHE0000267 | PMON68867 | 2/5 | 2/2 |
| 196 | 535 | PHE0000270 | PMON84751 | 2/4 | 0/0 |
| 197 | 536 | PHE0000271 | PMON84981 | 3/9 | 0/0 |
| 204 | 543 | PHE0000279 | PMON68896 | 2/3 | 2/3 |
| 205 | 544 | PHE0000280 | PMON72451 | 2/2 | 0/2 |
| 210 | 549 | PHE0000287 | PMON68898 | 1/2 | 0/0 |
| 214 | 553 | PHE0000291 | PMON72455 | 3/3 | 3/3 |
| 216 | 555 | PHE0000294 | PMON68897 | 2/3 | 0/0 |
| 217 | 556 | PHE0000295 | PMON68894 | 2/4 | 0/4 |
| 221 | 560 | PHE0000299 | PMON68875 | 1/2 | 0/2 |
| 223 | 562 | PHE0000301 | PMON68877 | 1/6 | 0/0 |
| 224 | 563 | PHE0000302 | PMON68878 | 1/1 | 0/0 |
| 227 | 566 | PHE0000305 | PMON68880 | 1/1 | 0/0 |
| 228 | 567 | PHE0000306 | PMON68882 | 1/1 | 0/0 |
| 234 | 573 | PHE0000312 | PMON72456 | 2/4 | 2/3 |
| 234 | 573 | PHE0000312 | PMON92811 | 11/11 | 0/0 |
| 235 | 574 | PHE0000313 | PMON68378 | 2/2 | 0/2 |
| 236 | 575 | PHE0000314 | PMON68379 | 4/4 | 4/4 |
| 237 | 576 | PHE0000315 | PMON68381 | 2/4 | 1/2 |
| 238 | 577 | PHE0000316 | PMON68382 | 1/3 | 1/2 |
| 239 | 578 | PHE0000317 | PMON68380 | 1/7 | 1/2 |
| 241 | 580 | PHE0000322 | PMON74403 | 1/1 | 0/0 |
| 243 | 582 | PHE0000324 | PMON73162 | 1/5 | 0/0 |
| 245 | 584 | PHE0000326 | PMON72463 | 1/1 | 0/0 |
| 246 | 585 | PHE0000327 | PMON69481 | 1/5 | 0/3 |
| 247 | 586 | PHE0000328 | PMON74416 | 1/4 | 0/4 |
| 249 | 588 | PHE0000330 | PMON73164 | 1/5 | 0/3 |
| 255 | 594 | PHE0000336 | PMON74414 | 2/4 | 0/0 |
| 262 | 601 | PHE0000345 | PMON74411 | 1/3 | 0/0 |
| 264 | 603 | PHE0000347 | PMON68386 | 2/2 | 1/2 |
| 266 | 605 | PHE0000350 | PMON74410 | 2/6 | 2/2 |
| 268 | 607 | PHE0000352 | PMON74409 | 3/5 | 1/5 |
| 269 | 608 | PHE0000353 | PMON73160 | 2/4 | 2/2 |
| 269 | 608 | PHE0000353 | PMON92582 | 3/8 | 0/0 |
| 270 | 609 | PHE0000354 | PMON81879 | 2/7 | 1/6 |
| 272 | 611 | PHE0000356 | PMON72464 | 1/4 | 0/0 |
| 284 | 623 | PHE0000390 | PMON67836 | 1/2 | 1/2 |
| 286 | 625 | PHE0000392 | PMON76335 | 2/2 | 1/2 |
| 295 | 634 | PHE0000402 | PMON67833 | 1/3 | 0/1 |
| 298 | 637 | PHE0000412 | PMON67843 | 2/3 | 2/3 |
| 301 | 640 | PHE0000415 | PMON67846 | 2/5 | 2/5 |
| 302 | 641 | PHE0000416 | PMON67847 | 2/2 | 1/2 |
| 303 | 642 | PHE0000418 | PMON69497 | 3/4 | 2/4 |
| 304 | 643 | PHE0000419 | PMON67848 | 3/3 | 2/3 |
| 306 | 645 | PHE0000421 | PMON83760 | 1/8 | 0/0 |
| 312 | 651 | PHE0000428 | PMON74417 | 1/1 | 0/0 |
| 313 | 652 | PHE0000429 | PMON74418 | 1/2 | 0/2 |
| 321 | 660 | PHE0000437 | PMON68630 | 1/2 | 0/1 |
| 324 | 663 | PHE0000440 | PMON72473 | 3/6 | 2/5 |
| 325 | 664 | PHE0000441 | PMON72474 | 1/5 | 0/1 |
| 326 | 665 | PHE0000451 | PMON72475 | 1/3 | 0/0 |
| 327 | 666 | PHE0000452 | PMON72476 | 1/1 | 0/0 |
| 338 | 677 | PHE0000486 | PMON69496 | 3/5 | 0/0 |
| 339 | 678 | PHE0000017 | PMON68850 | 4/4 | 0/3 |

### Nitrogen Use Field Efficacy Assay

Level I. Transgenic plants provided by the present invention are planted in field without any nitrogen source being applied. Transgenic plants and control plants are grouped by genotype and construct with controls arranged randomly within genotype blocks. Each type of transgenic plants are tested by 3 replications and across 5 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus(P), Potassium(K), organic matter and pH to provide baseline values. P, K and micronutrients are applied based upon soil test recommendations. A list of recombinant DNA constructs which improved growth without any nitrogen source in transgenic plants is illustrated in Table 11.

TABLE 11

| | | | | | Confirmed |
|---|---|---|---|---|---|
| | | | | Positive | events/Actual |
| NUC | PEP | | | events/Total | events with |
| SEQ | SEQ | | | events | confirmation |
| ID | ID | PHE | Construct | screened | attempted |
| 34 | 373 | PHE0000040 | PMON92405 | 1/3 | 0/0 |
| 62 | 401 | PHE0000067 | PMON92814 | 1/3 | 0/0 |
| 61 | 400 | PHE0000292 | PMON93851 | 1/3 | 0/0 |
| 236 | 575 | PHE0000314 | PMON94123 | 2/3 | 0/0 |

Level II. Transgenic plants provided by the present invention are planted in field with three levels of nitrogen (N) fertilizer being applied, i.e. low level (0 N), medium level (80 lb/ac) and high level (180 lb/ac). Liquid 28% or 32% UAN (Urea, Ammonium Nitrogen) are used as the N source and apply by broadcast boom and incorporate with a field cultivator with rear rolling basket in the same direction as intended crop rows. Although there is no N applied to the 0 N treatment the soil should still be disturbed in the same fashion as the treated area. Transgenic plants and control plants are grouped by genotype and construct with controls arranged randomly within genotype blocks. Each type of transgenic plants is tested by 3 replications and across 4 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus(P), Potassium(K), organic matter and pH to provide baseline values. P, K and micronutrients are applied based upon soil test recommendations.

### B. Selection for Increased Yield

Many transgenic plants of this invention exhibit improved yield as compared to a control plant. Improved yield can result from enhanced seed sink potential, i.e. the number and size of endosperm cells or kernels and/or enhanced sink strength, i.e. the rate of starch biosynthesis. Sink potential can be established very early during kernel development, as endosperm cell number and size are determined within the first few days after pollination.

Much of the increase in corn yield of the past several decades has resulted from an increase in planting density. During that period, corn yield has been increasing at a rate of 2.1 bushels/acre/year, but the planting density has increased at a rate of 250 plants/acre/year. A characteristic of modern hybrid corn is the ability of these varieties to be planted at high density. Many studies have shown that a higher than current planting density should result in more biomass production, but current germplasm does not perform. well at these higher densities. One approach to increasing yield is to increase harvest index (HI), the proportion of biomass that is allocated to the kernel compared to total biomass, in high density plantings.

Effective yield selection of enhanced yielding transgenic corn events uses hybrid progeny of the transgenic event over multiple locations with plants grown under optimal production management practices, and maximum pest control. A useful target for improved yield is a 5% to 10% increase in yield as compared to yield produced by plants grown from seed for a control plant. Selection methods may be applied in multiple and diverse geographic locations, for example up to 16 or more locations, over one or more plating seasons, for example at least two planting seasons to statistically distinguish yield improvement from natural environmental effects. It is to plant multiple transgenic plants, positive and negative control plants, and pollinator plants in standard plots, for example 2 row plots, 20 feet long by 5 feet wide with 30 inches distance between rows and a 3 foot alley between ranges. Transgenic events can be grouped by recombinant DNA constructs with groups randomly placed in the field. A pollinator plot of a high quality corn line is planted for every two plots to allow open pollination when using male sterile transgenic events. A useful planting density is about 30,000 plants/acre. High planting density is greater than 30,000 plants/acre, preferably about 40,000 plants/acre, more preferably about 42,000 plants/acre, most preferably about 45,000 plants/acre. Surrogate indicators for yield improvement include source capacity (biomass), source output (sucrose and photosynthesis), sink components (kernel size, ear size, starch in the seed), development (light response, height, density tolerance), maturity, early flowering trait and physiological responses to high density planting, for example at 45,000 plants per acre, for example as illustrated in Table 12 and 13.

TABLE 12

| Timing | Evaluation | Description | comments |
|---|---|---|---|
| V2-3 | Early stand | Can be taken any time after | |
| | | germination and prior to | |
| | | removal of any plants. | |
| Pollen | GDU to | GDU to 50% plants shedding | |
| shed | 50% shed | 50% tassel. | |
| Silking | GDU to | GDU to 50% plants showing | |
| | 50% silk | silks. | |
| Maturity | Plant height | Height from soil surface to | 10 plants per plot- |
| | | flag leaf attachment (inches). | Yield team assistance |
| Maturity | Ear height | Height from soil surface to | 10 plants per plot- |
| | | primary ear attachment node. | Yield team assistance |
| Maturity | Leaves | visual scores: erect, size, | |
| | above ear | rolling | |
| Maturity | Tassel size | Visual scores +/− vs. WT | |
| Pre- | Final Stand | Final stand count prior to | |
| Harvest | | harvest, exclude tillers | |
| Pre- | Stalk | No. of stalks broken below | |
| Harvest | lodging | the primary ear attachment. | |
| | | Exclude leaning tillers | |
| Pre- | Root | No. of stalks leaning >45° | |
| Harvest | lodging | angle from perpendicular. | |
| Pre- | Stay green | After physiological maturity | |
| Harvest | | and when differences among | |
| | | genotypes are evident: Scale | |
| | | 1 (90-100% tissue green)- | |
| | | 9 (0-19% tissue green). | |
| Harvest | Grain Yield | Grain yield/plot (Shell | |
| | | weight) | |

TABLE 13

| Timing | Evaluation | Description |
|---|---|---|
| V8-V12 | Chlorophyll | |
| V12-VT | Ear leaf area | |
| V15-15DAP | Chl fluorescence | |
| V15-15DAP | CER | |
| 15-25 DAP | Carbohydrates | sucrose, starch |
| Pre-Harvest | 1st internode diameter | |
| Pre-Harvest | Base 3 internode diameter | |
| Pre-Harvest | Ear internode diameter | |
| Maturity | Ear traits | diameter, length, kernel |
| | | number, kernel weight |

Electron transport rates (ETR) and CO2 exchange rates (CER): ETR and CER are measured with Li6400LCF (Licor, Lincoln, Nebr.) around V9-R1 stages. Leaf chlorophyll fluorescence is a quick way to monitor the source activity and is reported to be highly correlated with CO₂ assimilation under varies conditions (Photosyn Research, 37: 89-102). The youngest fully expanded leaf or 2 leaves above the car leaf is measured with actinic light 1500 (with 10% blue light) micromol m⁻² s⁻¹, 28° C. CO2 levels 450 ppm. Ten plants are measured in each event. There are 2 readings for each plant.

A hand-held chlorophyll meter SPAD-502 (Minolta—Japan) is used to measure the total chlorophyll level on live transgenic plants and the wild type counterparts a. Three trifoliates from each plant are analyzed, and each trifoliate were analyzed three times. Then 9 data points are averaged to obtain the chlorophyll level. The number of analyzed plants of each genotype ranges from 5 to 8.

When selecting for yield improvement a useful statistical measurement approach comprises three components, i.e. modeling spatial autocorrelation of the test field separately for each location, adjusting traits of recombinant DNA events for spatial dependence for each location, and conducting an across location analysis. The first step in modeling spatial autocorrelation is estimating the covariance parameters of the semivariogram. A spherical covariance model is assumed to model the spatial autocorrelation. Because of the size and nature of the trial, it is likely that the spatial autocorrelation may change. Therefore, anisotropy is also assumed along with spherical covariance structure. The following set of equations describes the statistical form of the anisotropic spherical covariance model.

$$C ( h ; θ ) = vI ( h = 0 ) + {σ}^{2} ( 1 - \frac{3}{2} h + \frac{1}{2} {h}^{3} ) I ( h < 1 ) ,$$

where I(•) is the indicator function, h=\sqrt{\dot{x}²+\dot{y}²}, and

\dot{x}=[cos(ρπ/180)(x₁−x₂)−sin(ρπ/180)(y₁−y₂)]/ωₓ

\dot{y}=[sin(ρπ/180)(x₁−x₂)+cos(ρπ/180)(y₁−y₂)]/ω_{y}

where s₁=(x₁, y₁) are the spatial coordinates of one location and s₂=(x₂, y₂) are the spatial coordinates of the second location. There are 5 covariance parameters, θ=(v,σ²,ρ,ωₙ,ωⱼ), where v is the nugget effect, σ² is the partial sill, ρ is a rotation in degrees clockwise from north, ωₙ is a scaling parameter for the minor axis and ωⱼ is a scaling parameter for the major axis of an anisotropical ellipse of equal covariance. The five covariance parameters that defines the spatial trend will then be estimated by using data from heavily replicated pollinator plots via restricted maximum likelihood approach. In a multi-location field trial, spatial trend are modeled separately for each location.

After obtaining the variance parameters of the model, a variance-covariance structure is generated for the data set to be analyzed. This variance-covariance structure contains spatial information required to adjust yield data for spatial dependence. In this case, a nested model that best represents the treatment and experimental design of the study is used along with the variance-covariance structure to adjust the yield data. During this process the nursery or the seed batch effects can also be modeled and estimated to adjust the yields for any yield parity caused by seed batch differences. After spatially adjusted data from different locations are generated, all adjusted data is combined and analyzed assuming locations as replications. In this analysis, intra and inter-location variances are combined to estimate the standard error of yield from transgenic plants and control plants. Relative mean comparisons are used to indicate statistically significant yield improvements. A list of recombinant DNA constructs which show improved yield in transgenic plants is illustrated in Table 14.

TABLE 14

| | | | | | Confirmed |
|---|---|---|---|---|---|
| | | | | Positive | events/ |
| | | | | events/ | Actual events |
| NUC | PEP | | | Total | with |
| SEQ | SEQ | | | events | confirmation |
| ID | ID | PHE ID | Construct | screened | attempted |
| 12 | 351 | PHE0000016 | PMON67750 | 1/4 | 0/2 |
| 14 | 353 | PHE0000019 | PMON80879 | 1/3 | 0/0 |
| 15 | 354 | PHE0000020 | PMON81241 | 1/8 | 0/0 |
| 31 | 370 | PHE0000034 | PMON67805 | 1/6 | 0/4 |
| 32 | 371 | PHE0000038 | PMON68383 | 1/7 | 0/0 |
| 33 | 372 | PHE0000039 | PMON67807 | 1/3 | 0/2 |
| 41 | 380 | PHE0000245 | PMON68373 | 1/4 | 0/1 |
| 42 | 381 | PHE0000246 | PMON68374 | 1/3 | 0/2 |
| 43 | 382 | PHE0000247 | PMON68375 | 1/4 | 0/2 |
| 68 | 407 | PHE0000073 | PMON68357 | 1/6 | 0/5 |
| 72 | 411 | PHE0000077 | PMON67827 | 2/8 | 1/4 |
| 95 | 434 | PHE0000108 | PMON67849 | 1/4 | 0/3 |
| 101 | 440 | PHE0000116 | PMON68367 | 1/7 | 0/6 |
| 102 | 441 | PHE0000117 | PMON68368 | 1/2 | 0/1 |
| 103 | 442 | PHE0000118 | PMON67811 | 1/7 | 0/4 |
| 105 | 444 | PHE0000120 | PMON68853 | 1/6 | 0/2 |
| 112 | 451 | PHE0000127 | PMON68887 | 2/5 | 0/3 |
| 116 | 455 | PHE0000153 | PMON67817 | 1/6 | 0/5 |
| 117 | 456 | PHE0000154 | PMON67818 | 1/3 | 1/2 |
| 123 | 462 | PHE0000161 | PMON82231 | 1/4 | 0/0 |
| 135 | 474 | PHE0000177 | PMON68881 | 1/3 | 0/2 |
| 136 | 475 | PHE0000178 | PMON73166 | 1/2 | 0/1 |
| 143 | 482 | PHE0000185 | PMON69468 | 1/4 | 1/2 |
| 146 | 485 | PHE0000188 | PMON73167 | 1/4 | 0/4 |
| 148 | 487 | PHE0000192 | PMON68394 | 1/7 | 0/5 |
| 214 | 553 | PHE0000291 | pMON72455 | 1/3 | 0/3 |
| 230 | 569 | PHE0000308 | PMON68884 | 2/3 | 0/1 |
| 257 | 596 | PHE0000338 | PMON68628 | 1/2 | 0/2 |
| 263 | 602 | PHE0000346 | PMON73165 | 1/3 | 0/2 |
| 264 | 603 | PHE0000347 | PMON68386 | 1/2 | 0/2 |
| 265 | 604 | PHE0000349 | PMON68389 | 1/4 | 1/1 |
| 280 | 619 | PHE0000386 | PMON67834 | 1/3 | 0/3 |
| 303 | 642 | PHE0000418 | PMON69497 | 1/4 | 0/2 |
| 326 | 665 | PHE0000451 | PMON72475 | 1/3 | 0/0 |

### C. Selection for Enhanced Water Use Efficiency (WUE)

Described in this example is a high-throughput method for greenhouse selection of transgenic corn plants to wild type corn plants (tested as inbreds or hybrids) for water use efficiency. This selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment. The hydration status of the shoot tissues following the drought is also measured. The plant height are measured at three time points. The first is taken just prior to the onset drought when the plant is 11 days old, which is the shoot initial height (SIH). The plant height is also measured halfway throughout the drought/re-water regimen, on day 18 after planting, to give rise to the shoot mid-drought height (SMH). Upon the completion of the final drought cycle on day 26 after planting, the shoot portion of the plant is harvested and measured for a final height, which is the shoot wilt height (SWH) and also measured for shoot wilted biomass (SWM). The shoot is placed in water at 40 degree Celsius in the dark. Three days later, the shoot is weighted to give rise to the shoot turgid weight (STM). After drying in an oven for four days, the shoots are weighted for shoot dry biomass (SDM). The shoot average height (SAH) is the mean plant height across the 3 height measurements. The procedure described above may be adjusted for +/−˜one day for each step given the situation.

To correct for slight differences between plants, a size corrected growth value is derived from SIH and SWH. This is the Relative Growth Rate (RGR). Relative Growth Rate (RGR) is calculated for each shoot using the formula [RGR %=(SWH−SIH)/((SWH+SIH)/2)*100]. Relative water content (RWC) is a measurement of how much (%) of the plant was water at harvest. Water Content (RWC) is calculated for each shoot using the formula [RWC %=(SWM−SDM)/(STM−SDM)*100]. Fully watered corn plants of this age run around 98% RWC. A list of recombinant DNA constructs which improved water use efficiency in transgenic plants is illustrated in Table 15.

TABLE 15

| | | | | | Confirmed |
|---|---|---|---|---|---|
| | | | | Positive | events/Actual |
| NUC | PEP | | | events/Total | events with |
| SEQ | SEQ | | | events | confirmation |
| ID | ID | PHE | Construct | screened | attempted |
| 2 | 341 | PHE0000006 | PMON68861 | 3/5 | 0/4 |
| 5 | 344 | PHE0000010 | PMON67800 | 2/5 | 0/4 |
| 8 | 347 | PHE0000012 | PMON67806 | 4/9 | 1/8 |
| 12 | 351 | PHE0000016 | PMON67750 | 3/4 | 1/4 |
| 15 | 354 | PHE0000020 | PMON81241 | 2/8 | 0/0 |
| 16 | 355 | PHE0000022 | PMON67826 | 2/3 | 1/2 |
| 17 | 356 | PHE0000024 | PMON68354 | 5/7 | 1/5 |
| 20 | 359 | PHE0000227 | PMON68376 | 3/5 | 0/4 |
| 23 | 362 | PHE0000049 | PMON80912 | 1/5 | 0/0 |
| 31 | 370 | PHE0000034 | PMON67805 | 4/7 | 0/7 |
| 32 | 371 | PHE0000038 | PMON68383 | 1/8 | 0/1 |
| 33 | 372 | PHE0000039 | PMON67807 | 2/3 | 0/2 |
| 34 | 373 | PHE0000040 | PMON67801 | 3/5 | 0/5 |
| 34 | 373 | PHE0000040 | PMON77889 | 1/4 | 0/0 |
| 37 | 376 | PHE0000045 | PMON81293 | 1/8 | 0/4 |
| 41 | 380 | PHE0000245 | PMON68373 | 2/5 | 1/3 |
| 42 | 381 | PHE0000246 | PMON68374 | 2/3 | 1/2 |
| 43 | 382 | PHE0000247 | PMON68375 | 3/4 | 1/2 |
| 46 | 385 | PHE0000051 | PMON68859 | 2/4 | 1/2 |
| 47 | 386 | PHE0000052 | PMON67813 | 3/5 | 0/5 |
| 48 | 387 | PHE0000382 | PMON74401 | 1/3 | 0/3 |
| 51 | 390 | PHE0000055 | PMON68355 | 1/3 | 1/3 |
| 53 | 392 | PHE0000057 | PMON68350 | 4/4 | 1/4 |
| 54 | 393 | PHE0000058 | PMON68351 | 2/3 | 1/2 |
| 56 | 395 | PHE0000060 | PMON68356 | 3/4 | 2/3 |
| 61 | 400 | PHE0000292 | PMON68888 | 2/2 | 0/2 |
| 62 | 401 | PHE0000067 | PMON67816 | 2/4 | 0/3 |
| 64 | 403 | PHE0000069 | PMON67821 | 4/5 | 0/5 |
| 65 | 404 | PHE0000070 | PMON67825 | 3/3 | 1/3 |
| 67 | 406 | PHE0000072 | PMON67828 | 2/2 | 2/2 |
| 68 | 407 | PHE0000073 | PMON68357 | 6/9 | N/A |
| 72 | 411 | PHE0000077 | PMON67827 | 1/6 | 1/5 |
| 74 | 413 | PHE0000079 | PMON67752 | 5/5 | 1/5 |
| 79 | 418 | PHE0000086 | PMON67812 | 3/5 | 0/0 |
| 83 | 422 | PHE0000092 | PMON68359 | 6/7 | 0/4 |
| 95 | 434 | PHE0000108 | PMON67849 | 3/4 | 1/4 |
| 99 | 438 | PHE0000114 | PMON68361 | 1/2 | 0/1 |
| 101 | 440 | PHE0000116 | PMON68367 | 3/7 | 0/7 |
| 102 | 441 | PHE0000117 | PMON38368 | 1/2 | 1/2 |
| 103 | 442 | PHE0000118 | PMON67811 | 5/7 | 3/6 |
| 104 | 443 | PHE0000119 | PMON68363 | 2/4 | 1/2 |
| 105 | 444 | PHE0000120 | PMON68853 | 2/6 | 0/2 |
| 108 | 447 | PHE0000123 | PMON68855 | 2/4 | 0/3 |
| 110 | 449 | PHE0000125 | PMON68369 | 2/7 | 0/3 |
| 111 | 450 | PHE0000126 | PMON69458 | 1/6 | 0/6 |
| 112 | 451 | PHE0000127 | PMON68887 | 1/5 | 0/4 |
| 114 | 453 | PHE0000133 | PMON68860 | 3/4 | 0/4 |
| 115 | 454 | PHE0000152 | PMON77899 | 1/7 | 0/4 |
| 116 | 455 | PHE0000153 | PMON67817 | 3/6 | 1/6 |
| 117 | 456 | PHE0000154 | PMON67818 | 2/3 | 2/2 |
| 123 | 462 | PHE0000161 | PMON82231 | 2/4 | 0/0 |
| 124 | 463 | PHE0000162 | PMON75488 | 2/6 | 0/0 |
| 129 | 468 | PHE0000168 | PMON68857 | 1/5 | 0/2 |
| 134 | 473 | PHE0000176 | PMON68388 | 1/4 | 0/2 |
| 135 | 474 | PHE0000177 | PMON68881 | 1/3 | 0/2 |
| 136 | 475 | PHE0000178 | PMON73166 | 2/2 | 0/2 |
| 143 | 482 | PHE0000185 | PMON69468 | 3/4 | 0/3 |
| 144 | 483 | PHE0000186 | PMON69460 | 2/2 | 1/1 |
| 146 | 485 | PHE0000188 | PMON73167 | 1/4 | 0/4 |
| 148 | 487 | PHE0000192 | PMON68394 | 6/7 | 0/1 |
| 169 | 508 | PHE0000235 | PMON73161 | 2/2 | 0/2 |
| 170 | 509 | PHE0000237 | PMON68891 | 2/2 | 0/2 |
| 171 | 510 | PHE0000238 | PMON69466 | 3/3 | 0/3 |
| 172 | 511 | PHE0000239 | PMON72466 | 1/5 | 1/4 |
| 177 | 516 | PHE0000249 | PMON74422 | 1/2 | 0/0 |
| 180 | 519 | PHE0000252 | PMON74407 | 1/4 | 0/0 |
| 186 | 525 | PHE0000260 | PMON75487 | 2/6 | 0/0 |
| 190 | 529 | PHE0000264 | PMON68866 | 2/3 | 1/3 |
| 193 | 532 | PHE0000267 | PMON68867 | 1/5 | 1/3 |
| 203 | 542 | PHE0000277 | PMON68890 | 1/2 | 0/1 |
| 204 | 543 | PHE0000279 | PMON68896 | 2/3 | 0/2 |
| 210 | 549 | PHE0000287 | PMON68898 | 2/3 | 0/2 |
| 214 | 553 | PHE0000291 | PMON72455 | 1/3 | 0/3 |
| 216 | 555 | PHE0000294 | PMON68897 | 1/3 | 0/0 |
| 217 | 556 | PHE0000295 | PMON68894 | 2/2 | 0/2 |
| 219 | 558 | PHE0000297 | PMON68899 | 2/4 | 0/4 |
| 221 | 560 | PHE0000299 | PMON68875 | 1/2 | 1/2 |
| 223 | 562 | PHE0000301 | PMON68877 | 2/6 | 0/5 |
| 228 | 567 | PHE0000306 | PMON68882 | 1/1 | 0/1 |
| 233 | 572 | PHE0000311 | PMON72458 | 1/1 | 0/0 |
| 234 | 573 | PHE0000312 | PMON72456 | 2/4 | 0/4 |
| 235 | 574 | PHE0000313 | PMON68378 | 1/3 | 1/2 |
| 236 | 575 | PHE0000314 | PMON68379 | 2/4 | 2/4 |
| 237 | 576 | PHE0000315 | PMON68381 | 1/4 | 0/4 |
| 238 | 577 | PHE0000316 | PMON68382 | 1/4 | 0/3 |
| 239 | 578 | PHE0000317 | PMON68380 | 5/5 | 1/5 |
| 241 | 580 | PHE0000322 | PMON74403 | 1/1 | 1/1 |
| 242 | 581 | PHE0000323 | PMON68400 | 1/7 | 0/0 |
| 243 | 582 | PHE0000324 | PMON73162 | 4/5 | 1/5 |
| 245 | 584 | PHE0000326 | PMON72463 | 2/5 | 1/5 |
| 246 | 585 | PHE0000327 | PMON69481 | 1/5 | 0/5 |
| 247 | 586 | PHE0000328 | PMON74416 | 2/4 | 0/4 |
| 249 | 588 | PHE0000330 | PMON73164 | 1/5 | 0/5 |
| 251 | 590 | PHE0000332 | PMON68385 | 1/3 | 0/1 |
| 252 | 591 | PHE0000333 | PMON75470 | 1/6 | 0/0 |
| 253 | 592 | PHE0000334 | PMON68395 | 2/9 | 0/2 |
| 262 | 601 | PHE0000345 | PMON74411 | 6/8 | 2/8 |
| 263 | 602 | PHE0000346 | PMON73165 | 1/3 | 0/3 |
| 264 | 603 | PHE0000347 | PMON68386 | 1/2 | 0/1 |
| 265 | 604 | PHE0000349 | PMON68389 | 1/2 | 0/2 |
| 266 | 605 | PHE0000350 | PMON74410 | 1/6 | 0/6 |
| 268 | 607 | PHE0000352 | PMON74409 | 1/5 | 0/5 |
| 269 | 608 | PHE0000353 | PMON73160 | 4/4 | 3/4 |
| 272 | 611 | PHE0000356 | PMON72464 | 2/4 | 0/3 |
| 280 | 619 | PHE0000386 | PMON67834 | 1/3 | 0/0 |
| 294 | 633 | PHE0000401 | PMON67837 | 4/5 | 0/0 |
| 301 | 640 | PHE0000415 | PMON67846 | 1/5 | 0/0 |
| 303 | 642 | PHE0000418 | PMON69497 | 2/4 | 0/0 |
| 304 | 643 | PHE0000419 | PMON67848 | 2/3 | 0/0 |
| 310 | 649 | PHE0000426 | PMON74408 | 1/5 | 0/0 |
| 313 | 652 | PHE0000429 | PMON74418 | 2/3 | 0/2 |
| 339 | 678 | PHE0000017 | PMON68850 | 3/4 | 1/4 |

### D. Selection for Growth Under Cold Stress

(1) Cold germination assay—Three sets of seeds are used for the assay. The first set consists of positive transgenic events (F1 hybrid) where the genes of the present invention are expressed in the seed. The second seed set is nontransgenic, wild-type negative control made from the same genotype as the transgenic events. The third set consisted of two cold tolerant and one cold sensitive commercial check lines of corn. All seeds are treated with a fungicide “Captan” (MAESTRO® 80DF Fungicide, Arvesta Corporation, San Francisco, Calif., USA). 0.43 mL Captan is applied per 45 g of corn seeds by mixing it well and drying the fungicide prior to the experiment.

Corn kernels are placed embryo side down on blotter paper within an individual cell (8.9×8.9 cm) of a germination tray (54×36 cm). Ten seeds from an event are placed into one cell of the germination tray. Each tray can hold 21 transgenic events and 3 replicates of wildtype (LH244SDms+LH59), which is randomized in a complete block design. For every event there are five replications (five trays). The trays are placed at 9.7 C for 24 days (no light) in a Convrion growth chamber (Conviron Model PGV36. Controlled Environments, Winnipeg. Canada). Two hundred and fifty milliliters of deionized water are added to each germination tray. Germination counts are taken 10th, 11th, 12th, 13th, 14th, 17th, 19th, 21st, and 24th day after start date of the experiment. Seeds are considered germinated if the emerged radicle size is 1 cm. From the germination counts germination index is calculated.

The germination index is calculated as per:

Germination index=(Σ([T+1−nᵢ]*[Pᵢ−Pᵢ₋₁]))/T

Where T is the total number of days for which the germination assay is performed. The number of days after planting is defined by n. “i” indicated the number of times the germination had been counted, including the current day. P is the percentage of seeds germinated during any given rating. Statistical differences are calculated between transgenic events and wild type control. After statistical analysis, the events that show a statistical significance at the p level of less than 0.1 relative to wild-type controls will advance to a secondary cold selection. The secondary cold screen is conducted in the same manner of the primary selection only increasing the number of repetitions to ten. Statistical analysis of the data from the secondary selection is conducted to identify the events that show a statistical significance at the p level of less than 0.05 relative to wild-type controls. A list of recombinant DNA constructs which improve growth in seed under cold stress in transgenic plants is illustrated in Table 16.

TABLE 16

| | | | | | Confirmed |
|---|---|---|---|---|---|
| | | | | Positive | events/ |
| | | | | events/ | Actual events |
| NUC | PEP | | | Total | with |
| SEQ | SEQ | | | events | confirmation |
| ID | ID | PHE | Construct | screened | attempted |
| 2 | 341 | PHE0000006 | PMON68861 | 1/4 | 0/1 |
| 5 | 344 | PHE0000010 | PMON67800 | 1/5 | 0/5 |
| 8 | 347 | PHE0000012 | PMON67808 | 3/7 | 0/3 |
| 12 | 351 | PHE0000016 | PMON67750 | 0/4 | 0/1 |
| 14 | 353 | PHE0000019 | PMON80879 | 1/8 | 0/0 |
| 16 | 355 | PHE0000022 | PMON67826 | 1/4 | 0/2 |
| 17 | 356 | PHE0000024 | PMON68354 | 1/7 | 0/5 |
| 29 | 368 | PHE0000032 | PMON83627 | 3/7 | 1/7 |
| 31 | 370 | PHE0000034 | PMON67805 | 5/7 | 4/6 |
| 33 | 372 | PHE0000039 | PMON67807 | 1/3 | 0/2 |
| 34 | 373 | PHE0000040 | PMON67801 | 2/5 | 1/4 |
| 34 | 373 | PHE0000040 | PMON92405 | 1/7 | 0/0 |
| 41 | 380 | PHE0000245 | PMON68373 | 1/3 | 0/2 |
| 42 | 381 | PHE0000246 | PMON68374 | 2/3 | 1/2 |
| 43 | 382 | PHE0000247 | PMON68375 | 2/4 | 0/2 |
| 44 | 383 | PHE0000106 | PMON92483 | 1/7 | 0/0 |
| 53 | 392 | PHE0000057 | PMON68350 | 3/4 | 1/3 |
| 56 | 395 | PHE0000060 | PMON68356 | 3/3 | 2/3 |
| 61 | 400 | PHE0000292 | PMON68888 | 1/2 | 0/2 |
| 62 | 401 | PHE0000067 | PMON67816 | 2/4 | 2/4 |
| 64 | 403 | PHE0000069 | PMON67821 | 1/5 | 0/3 |
| 68 | 407 | PHE0000073 | PMON68357 | 5/9 | 4/9 |
| 72 | 411 | PHE0000077 | PMON67827 | 1/6 | 0/5 |
| 74 | 413 | PHE0000079 | PMON67752 | 0/5 | 0/0 |
| 86 | 425 | PHE0000098 | PMON73168 | 1/2 | 0/0 |
| 92 | 431 | PHE0000104 | PMON68608 | 4/6 | 3/4 |
| 95 | 434 | PHE0000108 | PMON67849 | 1/4 | 0/2 |
| 101 | 440 | PHE0000116 | PMON68367 | 4/7 | 2/7 |
| 103 | 442 | PHE0000118 | PMON67811 | 5/7 | 2/6 |
| 105 | 444 | PHE0000120 | PMON68853 | 5/6 | 2/5 |
| 108 | 447 | PHE0000123 | PMON68855 | 1/5 | 0/3 |
| 109 | 448 | PHE0000124 | PMON68856 | 1/5 | 0/3 |
| 111 | 450 | PHE0000126 | PMON69458 | 2/7 | 1/7 |
| 112 | 451 | PHE0000127 | PMON68887 | 4/5 | 3/4 |
| 114 | 453 | PHE0000133 | PMON68860 | 3/4 | 0/4 |
| 115 | 454 | PHE0000152 | PMON77899 | 4/7 | 3/7 |
| 116 | 455 | PHE0000153 | PMON67817 | 6/6 | 5/6 |
| 117 | 456 | PHE0000154 | PMON67818 | 1/2 | 1/1 |
| 117 | 456 | PHE0000154 | PMON85035 | 1/7 | 0/0 |
| 120 | 459 | PHE0000158 | PMON73169 | 1/2 | 0/1 |
| 123 | 462 | PHE0000161 | PMON82231 | 1/4 | 0/0 |
| 124 | 463 | PHE0000162 | PMON75488 | 1/5 | 0/0 |
| 129 | 468 | PHE0000168 | PMON68857 | 3/5 | 2/3 |
| 133 | 472 | PHE0000173 | PMON73171 | 1/3 | 0/0 |
| 135 | 474 | PHE0000177 | PMON68881 | 1/3 | 0/2 |
| 136 | 475 | PHE0000178 | PMON73166 | 1/2 | 0/1 |
| 141 | 480 | PHE0000183 | PMON80258 | 3/5 | 0/5 |
| 143 | 482 | PHE0000185 | PMON69468 | 3/4 | 1/3 |
| 146 | 485 | PHE0000188 | PMON73167 | 1/4 | 1/2 |
| 148 | 487 | PHE0000192 | PMON68394 | 1/1 | 0/0 |
| 165 | 504 | PHE0000231 | PMON72498 | 3/7 | 2/7 |
| 168 | 507 | PHE0000234 | PMON73159 | 1/1 | 0/0 |
| 169 | 508 | PHE0000235 | PMON73161 | 2/2 | 0/2 |
| 170 | 509 | PHE0000237 | PMON68891 | 2/2 | 0/2 |
| 171 | 510 | PHE0000238 | PMON69466 | 3/3 | 0/3 |
| 172 | 511 | PHE0000239 | PMON72466 | 2/5 | 1/4 |
| 173 | 512 | PHE0000240 | PMON72468 | 3/5 | 1/5 |
| 182 | 521 | PHE0000254 | PMON73172 | 1/6 | 0/0 |
| 190 | 529 | PHE0000264 | PMON68866 | 4/4 | 3/4 |
| 191 | 530 | PHE0000265 | PMON69469 | 1/1 | 0/0 |
| 192 | 531 | PHE0000266 | PMON69470 | 3/4 | 2/3 |
| 193 | 532 | PHE0000267 | PMON68867 | 2/6 | 1/4 |
| 196 | 535 | PHE0000270 | PMON84751 | 1/5 | 0/1 |
| 199 | 538 | PHE0000273 | PMON74423 | 1/2 | 0/0 |
| 204 | 543 | PHE0000279 | PMON68896 | 1/3 | 0/2 |
| 210 | 549 | PHE0000287 | PMON68898 | 3/4 | 1/2 |
| 214 | 553 | PHE0000291 | PMON72455 | 3/3 | 2/3 |
| 217 | 556 | PHE0000295 | PMON68894 | 3/4 | 0/2 |
| 219 | 558 | PHE0000297 | PMON68899 | 1/4 | 1/3 |
| 220 | 559 | PHE0000298 | PMON68874 | 2/5 | 1/3 |
| 230 | 569 | PHE0000308 | PMON68884 | 3/3 | 2/2 |
| 234 | 573 | PHE0000312 | PMON72456 | 1/4 | 1/3 |
| 234 | 573 | PHE0000312 | PMON92811 | 2/7 | 0/7 |
| 236 | 575 | PHE0000314 | PMON68379 | 1/4 | 0/3 |
| 237 | 576 | PHE0000315 | PMON68381 | 2/4 | 0/2 |
| 239 | 578 | PHE0000317 | PMON68380 | 3/7 | 1/7 |
| 242 | 581 | PHE0000323 | PMON68400 | 4/5 | 2/5 |
| 246 | 585 | PHE0000327 | PMON69481 | 1/5 | 1/3 |
| 247 | 586 | PHE0000328 | PMON74416 | 2/6 | 1/2 |
| 249 | 588 | PHE0000330 | PMON73164 | 3/5 | 1/5 |
| 252 | 591 | PHE0000333 | PMON75470 | 2/3 | 0/0 |
| 253 | 592 | PHE0000334 | PMON68395 | 4/9 | 1/5 |
| 254 | 593 | PHE0000335 | PMON74413 | 1/6 | 0/2 |
| 260 | 599 | PHE0000341 | PMON68397 | 2/2 | 0/0 |
| 262 | 601 | PHE0000345 | PMON74411 | 7/8 | 3/6 |
| 266 | 605 | PHE0000350 | PMON74410 | 1/6 | 0/3 |
| 268 | 607 | PHE0000352 | PMON74409 | 1/5 | 0/3 |
| 269 | 608 | PHE0000353 | PMON73160 | 4/4 | 3/4 |
| 272 | 611 | PHE0000356 | PMON72464 | 4/4 | 0/4 |
| 280 | 619 | PHE0000386 | PMON67834 | 1/3 | 0/0 |
| 295 | 634 | PHE0000402 | PMON67833 | 2/3 | 0/1 |
| 300 | 639 | PHE0000414 | PMON67845 | 1 | 0/0 |
| 306 | 645 | PHE0000421 | PMON83760 | 1/8 | 0/0 |
| 317 | 656 | PHE0000433 | PMON74424 | 1/2 | 0/0 |
| 324 | 663 | PHE0000440 | PMON72473 | 5/6 | 1/6 |
| 325 | 664 | PHE0000441 | PMON72474 | 2/5 | 1/5 |
| 328 | 667 | PHE0000453 | PMON92409 | 1/4 | 0/0 |
| 337 | 676 | PHE0000485 | PMON69498 | 4/7 | 2/7 |
| 338 | 677 | PHE0000486 | PMON69496 | 2/5 | 1/5 |

(2) Cold Shock assay—The experimental set-up for the cold shock assay is the same as described in the above cold germination assay except seeds were grown in potted media for the cold shock assay.

The desired numbers of 2.5″ square plastic pots are placed on flats (n=32, 4×8). Pots were filled with Metro Mix 200 soil-less media containing 19:6:12 fertilizer (6 lbs/cubic yard) (Metro Mix, Pots and Flat are obtained from Hummert International, Earth City, Mo.). After planting seeds, pots are placed in a growth chamber set at 23° C., relative humidity of 65% with 12 hour day and night photoperiod (300 uE/m2-min). Planted seeds are watered for 20 minute every other day by sub-irrigation and fiats were rotated every third day in a growth chamber for growing corn seedlings.

On the 10^{th} day after planting the transgenic positive and wild-type negative (WT) plants are positioned in flats in an alternating pattern. Chlorophyll fluorescence of plants is measured on the 10^{th} day during the dark period of growth by using a PAM-2000 portable fluorometer as per the manufacturer's instructions (Walz, Germany). After chlorophyll measurements, leaf samples from each event are collected for confirming the expression of genes of the present invention. For expression analysis six V1 leaf tips from each selection are randomly harvested. The flats are moved to a growth chamber set at 5° C. All other conditions such as humidity, day/night cycle and light intensity are held constant in the growth chamber. The flats are sub-irrigated every day after transfer to the cold temperature. On the 4^{th} day chlorophyll fluorescence is measured. Plants are transferred to normal growth conditions after six days of cold shock treatment and allowed to recover for the next three days. During this recovery period the length of the V3 leaf is measured on the 1^{st} and 3^{rd} days. After two days of recovery V2 leaf damage is determined visually by estimating percent of green V2 leaf.

Statistical differences in V3 leaf growth, V2 leaf necrosis and fluorescence during pre-shock and cold shock can be used for estimation of cold shock damage on corn plants. (3) Early seedling growth assay—Three sets of seeds are used for the experiment. The first set consists of positive transgenic events (F1 hybrid) where the genes of the present invention are expressed in the seed. The second seed set is nontransgenic, wild-type negative control made from the same genotype as the transgenic events. The third seed set consists of two cold tolerant and two cold sensitive commercial check lines of corn. All seeds are treated with a fungicide “Captan”, (3a,4,7,a-tetrahydro-2-[(trichloromethly)thio]-1H-isoindole-1,3(2H)-dione, Drex Chemical Co. Memphis, Tenn.).

Seeds are grown in germination paper for the early seedling growth assay. Three 12″×18″ pieces of germination paper (Anchor Paper #SD7606) are used for each entry in the test (three repetitions per transgenic event). The papers are wetted in a solution of 0.5% KNO₃ and 0.1% Thyram.

For each paper fifteen seeds are placed on the line evenly spaced down the length of the paper. The fifteen seeds are positioned on the paper such that the radical would grow downward, for example longer distance to the paper's edge. The wet paper is rolled up starting from one of the short ends. The paper is rolled evenly and tight enough to hold the seeds in place. The roll is secured into place with two large paper clips, one at the top and one at the bottom. The rolls are incubated in a growth chamber at 23° C. for three days in a randomized complete block design within an appropriate container. The chamber is set for 65% humidity with no light cycle. For the cold stress treatment the rolls are then incubated in a growth chamber at 12° C. for twelve days. The chamber is set for 65% humidity with no light cycle.

After the cold treatment the germination papers are unrolled and the seeds that did not germinate are discarded. The lengths of the radicle and coleoptile for each seed are measured through an automated imaging program that automatically collects and processes the images. The imaging program automatically measures the shoot length, root length, and whole seedling length of every individual seedling and then calculates the average of each roll.

After statistical analysis, the events that show a statistical significance at the p level of less than 0.1 relative to wild-type controls will advance to a secondary cold selection. The secondary cold selection is conducted in the same manner of the primary selection only increasing the number of repetitions to five. Statistical analysis of the data from the secondary selection is conducted to identify the events that show a statistical significance at the p level of less than 0.05 relative to wild-type controls.

### 4. Cold Field Efficacy Trial

This example sets forth a cold field efficacy trial to identify gene constructs that confer enhanced cold vigor at germination and early seedling growth under early spring planting field conditions in conventional-till and simulated no-till environments. Seeds are planted into the ground around two weeks before local farmers are beginning to plant corn so that a significant cold stress is exerted onto the crop, named as cold treatment. Seeds also are planted under local optimal planting conditions such that the crop has little or no exposure to cold condition, named as normal treatment. The cold field efficacy trials are carried out in five locations, including Glyndon Minn., Mason Mich., Monmouth Ill., Dayton Iowa, Mystic Conn. At each location, seeds are planted under both cold and normal conditions with 3 repetitions per treatment, 20 kernels per row and single row per plot. Seeds are planted 1.5 to 2 inch deep into soil to avoid muddy conditions. Two temperature monitors are set up at each location to monitor both air and soil temperature daily.

Seed emergence is defined as the point when the growing shoot breaks the soil surface. The number of emerged seedling in each plot is counted everyday from the day the earliest plot begins to emerge until no significant changes in emergence occur. In addition, for each planting date, the latest date when emergence is 0 in all plots is also recorded. Seedling vigor is also rated at V3-V4 stage before the average of corn plant height reaches 10 inches, with 1=excellent early growth, 5=Average growth and 9=poor growth. Days to 50% emergence, maximum percent emergence and seedling vigor are calculated using SAS software for the data within each location or across all locations.

A list of recombinant DNA constructs which enhanced cold vigor at germination and early seedling growth under early spring planting field conditions in table 17.

TABLE 17

| | | | | | Confirmed |
|---|---|---|---|---|---|
| | | | | Positive | events/ |
| | | | | events/ | Actual |
| NUC | PEP | | | Total | events with |
| SEQ | SEQ | | | events | confirmation |
| ID | ID | PHE | Construct | screened | attempted |
| 31 | 370 | PHE0000034 | PMON67805 | | 0/0 |
| 34 | 373 | PHE0000040 | PMON67801 | 1/5 | 0/0 |
| 92 | 431 | PHE0000104 | PMON68608 | 3/4 | 0/0 |
| 124 | 463 | PHE0000162 | PMON75488 | 1/4 | 0/0 |
| 129 | 468 | PHE0000168 | PMON68857 | 2/3 | 0/0 |
| 143 | 482 | PHE0000185 | PMON69468 | 2/3 | 0/0 |
| 165 | 504 | PHE0000231 | PMON72498 | 2/3 | 0/0 |
| 192 | 531 | PHE0000266 | PMON69470 | 2/2 | 0/0 |
| 242 | 581 | PHE0000323 | PMON68400 | 1/3 | 0/0 |
| 262 | 601 | PHE0000345 | PMON74411 | 4/4 | 0/0 |
| 269 | 608 | PHE0000353 | PMON73160 | 1/4 | 0/0 |
| 294 | 633 | PHE0000401 | PMON67837 | 1/3 | 0/0 |
| 310 | 649 | PHE0000426 | PMON74408 | 1/4 | 0/0 |
| 337 | 676 | PHE0000485 | PMON69498 | 2/3 | 0/0 |

E. Screens for Transgenic Plant Seeds with Increased Protein and/or Oil Levels

This example sets forth a high-throughput selection for identifying plant seeds with improvement in seed composition using the Infratec 1200 series Grain Analyzer, which is a near-infrared transmittance spectrometer used to determine the composition of a bulk seed sample. Near infrared analysis is a non-destructive, high-throughput method that can analyze multiple traits in a single sample scan. An NIR calibration for the analytes of interest is used to predict the values of an unknown sample. The NIR spectrum is obtained for the sample and compared to the calibration using a complex chemometric software package that provides a predicted values as well as information on how well the sample fits in the calibration.

Infratec Model 1221, 1225, or 1227 with transport module by Foss North America is used with cuvette, item #1000-4033, Foss North America or for small samples with small cell cuvette. Foss standard cuvette modified by Leon Girard Co. Corn and soy check samples of varying composition maintained in check cell cuvettes are supplied by Leon Girard Co. NIT collection software is provided by Maximum Consulting Inc. Software. Calculations are performed automatically by the software. Seed samples are received in packets or containers with barcode labels from the customer. The seed is poured into the cuvettes and analyzed as received. The detail information has been provided in Table 18.

TABLE 18

| | |
|---|---|
| Typical sample(s): | Whole grain corn and soybean seeds |
| Analytical time to run method: | Less than 0.75 min per sample |
| Total elapsed time per run: | 1.5 minute per sample |
| Typical and minimum sample size: | Corn typical: 50 cc; minimum 30 cc |
| | Soybean typical: 50 cc; minimum 5 cc |
| Typical analytical range: | Determined in part by the specific |
| | calibration. |
| | Corn-moisture 5-15%, oil 5-20%, |
| | protein 5-30%, starch 50-75%, and |
| | density 1.0-1.3%. |
| | Soybean-moisture 5-15%, oil 15-25%, |
| | and protein 35-50%. |

A list of recombinant DNA constructs which improve seed compositions in terms of protein content in transgenic plants is illustrated in Table 19.

TABLE 19

| | | | | | Confirmed |
|---|---|---|---|---|---|
| | | | | | events/ |
| | | | | Positive | Actual |
| | | | | events/ | events |
| NUC | PEP | | | Total | with |
| SEQ | SEQ | | | events | confirmation |
| ID | ID | PHE | Construct | screened | attempted |
| 2 | 341 | PHE0000006 | PMON68861 | 1/1 | 0/0 |
| 6 | 345 | PHE0000278 | PMON68886 | 1/1 | 0/0 |
| 8 | 347 | PHE0000012 | PMON57626 | 1/8 | 0/1 |
| 8 | 347 | PHE0000012 | PMON67806 | 2/3 | 0/4 |
| 8 | 347 | PHE0000012 | PMON67808 | 1/6 | 2/2 |
| 12 | 351 | PHE0000016 | PMON67750 | 1/3 | 2/2 |
| 20 | 359 | PHE0000227 | PMON68376 | 1/5 | 0/0 |
| 22 | 361 | PHE0000259 | PMON74404 | 2/5 | 1/1 |
| 29 | 368 | PHE0000032 | PMON83627 | 8/8 | 3/3 |
| 31 | 370 | PHE0000034 | PMON67805 | 1/6 | 0/0 |
| 33 | 372 | PHE0000039 | PMON67807 | 1/2 | 0/3 |
| 34 | 373 | PHE0000040 | PMON67801 | 1/5 | 0/2 |
| 37 | 376 | PHE0000045 | PMON81293 | 1/2 | 0/0 |
| 41 | 380 | PHE0000245 | PMON68373 | 1/2 | 1/2 |
| 42 | 381 | PHE0000246 | PMON68374 | 2/2 | 1/4 |
| 43 | 382 | PHE0000247 | PMON68375 | 2/3 | 1/2 |
| 44 | 383 | PHE0000106 | PMON69457 | 1/1 | 0/0 |
| 47 | 386 | PHE0000052 | PMON67813 | 1/5 | 0/0 |
| 53 | 392 | PHE0000057 | PMON68350 | 1/3 | 0/0 |
| 54 | 393 | PHE0000058 | PMON68351 | 2/4 | 0/4 |
| 56 | 395 | PHE0000060 | PMON68356 | 3/4 | 6/6 |
| 59 | 398 | PHE0000064 | PMON67804 | 1/6 | 0/0 |
| 61 | 400 | PHE0000292 | PMON68888 | 1/3 | 0/1 |
| 62 | 401 | PHE0000067 | PMON67816 | 3/4 | 0/0 |
| 64 | 403 | PHE0000069 | PMON67821 | 3/5 | 0/1 |
| 67 | 406 | PHE0000072 | PMON67828 | 1/2 | 0/0 |
| 68 | 407 | PHE0000073 | PMON68357 | 3/6 | 2/6 |
| 71 | 410 | PHE0000076 | PMON68851 | 2/2 | 1/2 |
| 72 | 411 | PHE0000077 | PMON67827 | 1/5 | 2/2 |
| 72 | 411 | PHE0000077 | PMON77890 | 1/2 | 0/0 |
| 74 | 413 | PHE0000079 | PMON67752 | 1/5 | 0/0 |
| 79 | 418 | PHE0000086 | PMON67812 | 3/5 | 2/3 |
| 82 | 421 | PHE0000091 | PMON68358 | 1/1 | 0/0 |
| 83 | 422 | PHE0000092 | PMON68359 | 2/6 | 0/0 |
| 86 | 425 | PHE0000098 | PMON73168 | 1/4 | 0/0 |
| 90 | 429 | PHE0000102 | PMON67815 | 1/2 | 0/0 |
| 92 | 431 | PHE0000104 | PMON68608 | 2/6 | 0/1 |
| 99 | 438 | PHE0000114 | PMON68361 | 2/2 | 0/2 |
| 101 | 440 | PHE0000116 | PMON68367 | 3/7 | 0/4 |
| 102 | 441 | PHE0000117 | PMON68368 | 2/2 | 0/2 |
| 103 | 442 | PHE0000118 | PMON67811 | 6/6 | 6/16 |
| 104 | 443 | PHE0000119 | PMON68363 | 3/4 | 3/6 |
| 105 | 444 | PHE0000120 | PMON68853 | 1/2 | 2/2 |
| 108 | 447 | PHE0000123 | PMON68855 | 4/4 | 2/2 |
| 110 | 449 | PHE0000125 | PMON68369 | 2/7 | 2/2 |
| 111 | 450 | PHE0000126 | PMON69458 | 2/8 | 1/1 |
| 112 | 451 | PHE0000127 | PMON68887 | 2/4 | 1/4 |
| 114 | 453 | PHE0000133 | PMON68860 | 1/4 | 0/0 |
| 115 | 454 | PHE0000152 | PMON77899 | 2/7 | 2/2 |
| 116 | 455 | PHE0000153 | PMON67817 | 4/6 | 0/0 |
| 117 | 456 | PHE0000154 | PMON67818 | 1/3 | 0/0 |
| 122 | 461 | PHE0000160 | PMON75485 | 1/1 | 0/0 |
| 124 | 463 | PHE0000162 | PMON75488 | 2/5 | 0/0 |
| 125 | 464 | PHE0000164 | PMON73170 | 2/2 | 0/0 |
| 129 | 468 | PHE0000168 | PMON68857 | 1/5 | 1/1 |
| 133 | 472 | PHE0000173 | PMON73171 | 2/4 | 0/0 |
| 134 | 473 | PHE0000176 | PMON68388 | 1/3 | 0/0 |
| 136 | 475 | PHE0000178 | PMON73166 | 1/2 | 0/0 |
| 138 | 477 | PHE0000180 | PMON83753 | 5/8 | 1/5 |
| 140 | 479 | PHE0000182 | PMON74420 | 1/3 | 1/1 |
| 143 | 482 | PHE0000185 | PMON69468 | 2/3 | 0/2 |
| 144 | 483 | PHE0000186 | PMON69460 | 1/2 | 0/0 |
| 146 | 485 | PHE0000188 | PMON73167 | 1/4 | 0/1 |
| 148 | 487 | PHE0000192 | PMON68394 | 1/7 | 0/1 |
| 149 | 488 | PHE0000193 | PMON68889 | 2/3 | 0/0 |
| 151 | 490 | PHE0000219 | PMON68865 | 1/3 | 0/0 |
| 155 | 494 | PHE0000220 | PMON74434 | 4/8 | 2/3 |
| 158 | 497 | PHE0000223 | PMON69478 | 1/1 | 1/1 |
| 165 | 504 | PHE0000231 | PMON72498 | 1/5 | 0/0 |
| 168 | 507 | PHE0000234 | PMON73159 | 1/1 | 0/0 |
| 170 | 509 | PHE0000237 | PMON68891 | 1/2 | 0/0 |
| 171 | 510 | PHE0000238 | PMON69466 | 1/3 | 0/0 |
| 172 | 511 | PHE0000239 | PMON72466 | 3/5 | 0/0 |
| 175 | 514 | PHE0000242 | PMON72470 | 1/3 | 1/1 |
| 180 | 519 | PHE0000252 | PMON74407 | 2/4 | 0/1 |
| 182 | 521 | PHE0000254 | PMON73172 | 1/4 | 0/1 |
| 186 | 525 | PHE0000260 | PMON75487 | 2/6 | 0/0 |
| 192 | 531 | PHE0000266 | PMON69470 | 1/3 | 0/3 |
| 193 | 532 | PHE0000267 | PMON68867 | 3/5 | 2/2 |
| 202 | 541 | PHE0000276 | PMON68868 | 1/1 | 0/0 |
| 203 | 542 | PHE0000277 | PMON68890 | 1/2 | 0/0 |
| 204 | 543 | PHE0000279 | PMON68896 | 1/1 | 0/0 |
| 204 | 543 | PHE0000279 | PMON68896 | 1/1 | 0/0 |
| 205 | 544 | PHE0000280 | PMON72451 | 1/3 | 0/0 |
| 214 | 553 | PHE0000291 | PMON85037 | 2/15 | 1/2 |
| 216 | 555 | PHE0000294 | PMON68897 | 2/3 | 1/1 |
| 217 | 556 | PHE0000295 | PMON68894 | 3/4 | 0/4 |
| 219 | 558 | PHE0000297 | PMON68899 | 1/3 | 0/0 |
| 220 | 559 | PHE0000298 | PMON68874 | 2/4 | 0/1 |
| 222 | 561 | PHE0000300 | PMON68876 | 1/3 | 0/1 |
| 223 | 562 | PHE0000301 | PMON68877 | 3/6 | 0/0 |
| 228 | 567 | PHE0000306 | PMON68882 | 1/1 | 0/0 |
| 230 | 569 | PHE0000308 | PMON68884 | 1/2 | 0/2 |
| 232 | 571 | PHE0000310 | PMON68377 | 2/2 | 0/0 |
| 233 | 572 | PHE0000311 | PMON72458 | 1/1 | 0/0 |
| 234 | 573 | PHE0000312 | PMON72456 | 4/4 | 2/3 |
| 236 | 575 | PHE0000314 | PMON68379 | 2/4 | 0/0 |
| 237 | 576 | PHE0000315 | PMON68381 | 1/4 | 0/0 |
| 238 | 577 | PHE0000316 | PMON68382 | 2/3 | 1/1 |
| 239 | 578 | PHE0000317 | PMON68380 | 2/7 | 0/0 |
| 243 | 582 | PHE0000324 | PMON73162 | 2/5 | 0/0 |
| 245 | 584 | PHE0000326 | PMON72463 | 1/5 | 0/0 |
| 247 | 586 | PHE0000328 | PMON74416 | 3/4 | 0/0 |
| 249 | 588 | PHE0000330 | PMON73164 | 2/5 | 0/0 |
| 252 | 591 | PHE0000333 | PMON75470 | 1/4 | 0/0 |
| 253 | 592 | PHE0000334 | PMON68395 | 117 | 0/0 |
| 255 | 594 | PHE0000336 | PMON74414 | 2/4 | 0/1 |
| 258 | 597 | PHE0000339 | PMON68627 | 1/1 | 0/0 |
| 262 | 601 | PHE0000345 | PMON74411 | 3/8 | 0/0 |
| 264 | 603 | PHE0000347 | PMON68386 | 2/2 | 0/2 |
| 266 | 605 | PHE0000350 | PMON74410 | 3/6 | 1/3 |
| 268 | 607 | PHE0000352 | PMON74409 | 1/5 | 0/0 |
| 269 | 608 | PHE0000353 | PMON73160 | 1/4 | 2/2 |
| 272 | 611 | PHE0000356 | PMON72464 | 2/4 | 0/0 |
| 280 | 619 | PHE0000386 | PMON67834 | 1/3 | 0/1 |
| 291 | 630 | PHE0000398 | PMON72488 | 1/2 | 0/0 |
| 296 | 635 | PHE0000403 | PMON67831 | 1/3 | 0/3 |
| 298 | 637 | PHE0000412 | PMON67843 | 2/4 | 0/0 |
| 300 | 639 | PHE0000414 | PMON67845 | 1/1 | 0/0 |
| 301 | 640 | PHE0000415 | PMON67846 | 1/5 | 0/1 |
| 303 | 642 | PHE0000418 | PMON69497 | 2/4 | 2/2 |
| 306 | 645 | PHE0000421 | PMON83760 | 6/8 | 1/1 |
| 309 | 648 | PHE0000425 | PMON72495 | 1/1 | 0/0 |
| 310 | 649 | PHE0000426 | PMON74408 | 2/5 | 0/0 |
| 312 | 651 | PHE0000428 | PMON74417 | 1/1 | 0/0 |
| 317 | 656 | PHE0000433 | PMON74424 | 2/2 | 0/1 |
| 321 | 660 | PHE0000437 | PMON68630 | 3/4 | 2/3 |
| 324 | 663 | PHE0000440 | PMON72473 | 4/6 | 0/0 |
| 325 | 664 | PHE0000441 | PMON72474 | 3/5 | 0/0 |
| 326 | 665 | PHE0000451 | PMON72475 | 1/2 | 0/1 |
| 329 | 668 | PHE0000454 | PMON72477 | 1/3 | 0/0 |
| 331 | 670 | PHE0000469 | PMON68636 | 1/3 | 0/1 |
| 338 | 677 | PHE0000486 | PMON69496 | 1/5 | 0/0 |
| 339 | 678 | PHE0000017 | PMON68850 | 1/4 | 0/0 |

A list of recombinant DNA constructs which improve seed compositions in terms of oil content in transgenic plants is illustrated in Table 20.

TABLE 20

| | | | | Positive | Confirmed |
|---|---|---|---|---|---|
| | | | | events/ | events/Actual |
| NUC | PEP | | | Total | events with |
| SEQ | SEQ | | | events | confirmation |
| ID | ID | PHE | Construct | screened | attempted |
| 2 | 341 | PHE0000006 | PMON68861 | 1/3 | 0/0 |
| 8 | 347 | PHE0000012 | PMON57626 | 1/2 | 0/0 |
| 8 | 347 | PHE0000012 | PMON67806 | 1/3 | 0/2 |
| 8 | 347 | PHE0000012 | PMON67808 | 1/6 | 2/4 |
| 12 | 351 | PHE0000016 | PMON67750 | 2/3 | 1/4 |
| 34 | 373 | PHE0000040 | PMON67801 | 1/5 | 0/2 |
| 34 | 373 | PHE0000040 | PMON77889 | 1/2 | 0/0 |
| 40 | 379 | PHE0000244 | PMON68372 | 1/1 | 1/2 |
| 41 | 380 | PHE0000245 | PMON68373 | 2/2 | 1/4 |
| 42 | 381 | PHE0000246 | PMON68374 | 1/2 | 0/2 |
| 43 | 382 | PHE0000247 | PMON68375 | 1/3 | 0/2 |
| 46 | 385 | PHE0000051 | PMON68859 | 1/3 | 0/0 |
| 47 | 386 | PHE0000052 | PMON67813 | 1/4 | 0/0 |
| 54 | 393 | PHE0000058 | PMON68351 | 1/3 | 0/3 |
| 56 | 395 | PHE0000060 | PMON68356 | 1/3 | 1/3 |
| 68 | 407 | PHE0000073 | PMON68357 | 2/6 | 0/4 |
| 71 | 410 | PHE0000076 | PMON68851 | 1/2 | 0/0 |
| 72 | 411 | PHE0000077 | PMON67827 | 1/5 | 1/2 |
| 101 | 440 | PHE0000116 | PMON68367 | 1/7 | 0/3 |
| 102 | 441 | PHE0000117 | PMON68368 | 1/2 | 0/2 |
| 103 | 442 | PHE0000118 | PMON67811 | 6/6 | 4/15 |
| 105 | 444 | PHE0000120 | PMON68853 | 1/2 | 1/2 |
| 108 | 447 | PHE0000123 | PMON68855 | 1/3 | 0/2 |
| 110 | 449 | PHE0000125 | PMON68369 | 1/3 | 0/0 |
| 111 | 450 | PHE0000126 | PMON69458 | 1/3 | 0/0 |
| 129 | 468 | PHE0000168 | PMON68857 | 1/4 | 0/0 |
| 169 | 508 | PHE0000235 | PMON73161 | 1/2 | 0/0 |
| 182 | 521 | PHE0000254 | PMON73172 | 1/2 | 0/0 |
| 193 | 532 | PHE0000267 | PMON68867 | 1/4 | 1/2 |
| 214 | 553 | PHE0000291 | PMON72455 | 1/3 | 0/0 |
| 216 | 555 | PHE0000294 | PMON68897 | 1/1 | 0/0 |
| 217 | 556 | PHE0000295 | PMON68894 | 1/2 | 0/2 |
| 219 | 558 | PHE0000297 | PMON68899 | 1/4 | 0/0 |
| 221 | 560 | PHE0000299 | PMON68875 | 1/1 | 0/0 |
| 222 | 561 | PHE0000300 | PMON68876 | 1/1 | 0/0 |
| 223 | 562 | PHE0000301 | PMON68877 | 1/6 | 0/0 |
| 238 | 577 | PHE0000316 | PMON68382 | 1/1 | 0/0 |
| 249 | 588 | PHE0000330 | PMON73164 | 2/5 | 0/0 |
| 269 | 608 | PHE0000353 | PMON73160 | 1/4 | 1/2 |
| 272 | 611 | PHE0000356 | PMON72464 | 1/4 | 0/0 |
| 296 | 635 | PHE0000403 | PMON67831 | 1/2 | 0/1 |
| 304 | 643 | PHE0000419 | PMON67848 | 1/2 | 0/0 |
| 321 | 660 | PHE0000437 | PMON68630 | 1/2 | 0/0 |
| 326 | 665 | PHE0000451 | PMON72475 | 1/1 | 0/0 |
| 327 | 666 | PHE0000452 | PMON72476 | 1/1 | 0/0 |

### Example 8

### Consensus Sequence

This example illustrates the identification of consensus amino acid sequence for the proteins and homologs encoded by DNA that is used to prepare the transgenic seed and plants of this invention having enhanced agronomic traits.

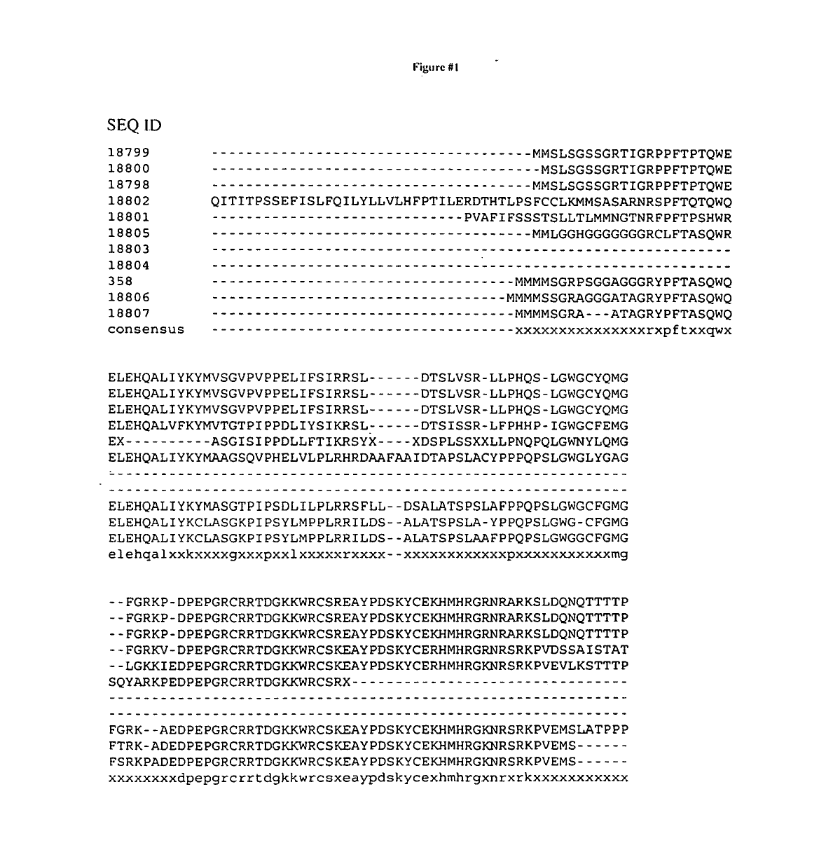

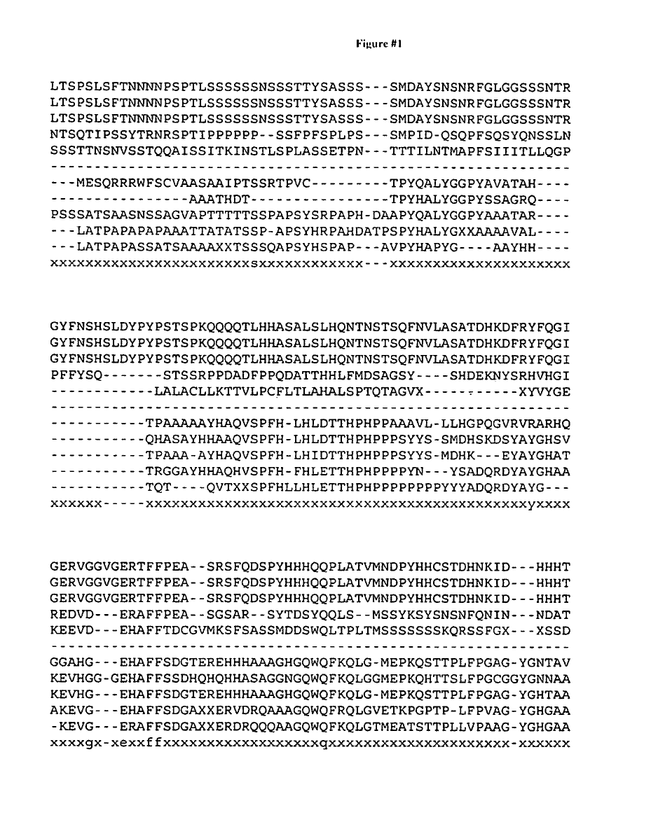

ClustalW program was selected for multiple sequence alignments of the amino acid sequence of SEQ ID NO: 357, 358, 369, 397, 468, 497, 508, 512, 514, 516, 518, 541, 551, 570, 578, 608, 645, 653, 658, 660, 668, 669 and their homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment. FIG. 1 shows the consensus sequence of SEQ ID NO: 358 and its homologs. The symbols for consensus sequence are (1) uppercase letters for 100% identity in all positions of multiple sequence alignment output; (2) lowercase letters for >=70% identity; symbol; (3)“X” indicated <70% identity; (4) dashes “-” meaning that gaps were in >=70% sequences.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA encoding a protein with amino acid sequence identical to the consensus amino acid sequence.

### Example 9

### Pfam Domain Module Annotation

This example illustrates the identification of domain and domain module by Pfam analysis.

The amino acid sequence of the expressed proteins that were shown to be associated with an enhanced trait were analyzed for Pfam protein family against the current Pfam collection of multiple sequence alignments and hidden Markov models using the HMMER software in the appended computer listing. The Pfam domain modules and individual protein domain for the proteins of SEQ ID NO: 340 through 678 are shown in Table 21 and Table 22 respectively. The Hidden Markov model databases for the identified protein families are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains. For instance, the protein with amino acids of SEQ ID NO: 401 is characterized by two Pfam domains, i.e KOW and eIF-5a. See also the protein with amino acids of SEQ ID NO: 346 which is characterized by two copies of the Pfam domain “AP2”. In Table 22 “score” is the gathering score for the Hidden Markov Model of the domain which exceeds the gathering cutoff reported in Table 23.

TABLE 21

| PEP | | |
|---|---|---|
| SEQ | | |
| ID NO | Pfam module annoation | pfam coordinates |
| 340 | Cellulose_synt | 167-977 |
| 341 | AP2::B3 | 67-129::192-300 |
| 342 | AP2::B3 | 66-128::181-294 |
| 343 | AP2::B3 | 64-126::177-286 |
| 344 | AP2 | 5-69 |
| 345 | AP2 | 13-77 |
| 346 | AP2::AP2 | 111-174::203-267 |
| 347 | MIP | 11-231 |
| 348 | Cyclin_N::Cyclin_C | 63-195::197-317 |
| 349 | Glyco_hydro_32N::Glyco_ | 118-438::479-601 |
| | hydro_32C | |
| 350 | Dicty_CAR | 12-328 |
| 351 | KNOX1::KNOX2::ELK:: | 102-146::153-204::242- |
| | Homeobox | 263::273-324 |
| 352 | CDC48_N::AAA::AAA | 30-116::247-431::520-707 |
| 353 | AOX | 55-330 |
| 354 | AOX | 26-333 |
| 355 | Aa_trans | 32-471 |
| 356 | PI3_PI4_kinase | 169-432 |
| 359 | FA_desaturase | 156-400 |
| 360 | FA_desaturase | 147-391 |
| 361 | FA_desaturase | 140-384 |
| 362 | PAS_2::GAF:: | 70-186::219-404::415-595::622- |
| | Phytochrome::PAS: | 737::752-877::897 |
| | :PAS::HisKA::HATPase_c | 956::1011-1123 |
| 363 | PAS_2::GAF:: | 70-186::219-404::415-595::622- |
| | Phylochrome::PAS: | 737::752-877::897- |
| | :PAS::HisKA::HATPase_c | 956::1011-1123 |
| 364 | PAS_2::GAF:: | 105-226::259-442::453-632::663- |
| | Phytochrome::PAS: | 779::794-916::936- |
| | :PAS::HisKA::HATPase_c | 1000::1048-1160 |
| 365 | PAS_2::GAF:: | 114-234::267-449::460-639::670- |
| | Phytochrome::PAS: | 786::801-923::943- |
| | :PAS::HisKA::HATPase_c | 1007:1055-1167 |
| 366 | PAS_2::GAF:: | 68-184::217-400::411-591::622- |
| | Phytochrome::PAS: | 737::752-877::898- |
| | :PAS::HisKA::HATPase_c | 961:1009-1121 |
| 367 | PAS_2::GAF:: | 67-183::216-399::410-590::620- |
| | Phytochrome::PAS: | 735::750-875::896- |
| | :PAS::HisKA::HATPase_c | 959::1007-1121 |
| 368 | Linker_histone::AT_ | 21-97::98-110::129- |
| | hook::AT_hoo | 141::154-166::192-204 |
| | k::AT_hook::AT_hook | |
| 370 | GFO_IDH_MocA::GFO_ | 11-129::130-236 |
| | IDH_MocA_C | |
| 371 | Cyclin_N::Cyclin_C | 54-186::188-314 |
| 372 | PAS_3::PAS_3::Pkinase | 141-233::415-507::582-870 |
| 373 | Globin | 17-157 |
| 374 | Cyclin_N::Cyclin_C | 165-291::293-413 |
| 375 | Cyclin_N | 4-144 |
| 376 | Cyclin_N::Cyclin_C | 157-283::285-405 |
| 377 | Cyclin_N::Cyclin_C | 243-370::372-499 |
| 378 | Cyclin_N::Cyclin_C | 166-292::294-415 |
| 379 | SRF-TF::K-box | 9-59::69-172 |
| 380 | SRF-TF::K-box | 13-63::73-178 |
| 381 | SRF-TF::K-box | 9-59::72-171 |
| 382 | SRF-TF::K-box | 9-59::73-171 |
| 383 | Cyclin_N::Cyclin_C | 244-371::373-500 |
| 384 | Cyclin_N::Cyclin_C | 104-233::235-363 |
| 385 | Cyclin_N::Cyclin_C | 163-289::291-411 |
| 386 | Cyclin_N::Cyclin_C | 228-354::356-477 |
| 387 | Cyclin_N::Cyclin_C | 173-299::301-421 |
| 388 | Cyclin_N::Cyclin_C | 187-312::314-441 |
| 389 | Cyclin_N | 47-190 |
| 390 | Cyclin_N::Cyclin_C | 43-176::178-298 |
| 391 | Cyclin_N | 55-184 |
| 392 | NDK | 75-209 |
| 393 | NDK | 89-223 |
| 394 | NDK | 2-134 |
| 395 | NDK | 2-135 |
| 396 | SNF2_N::Helicase_C | 560-842::891-970 |
| 398 | NDK | 33-170 |
| 399 | HEAT::HEAT::HEAT:: | 248-284::746-782::787- |
| | FAT::PI3_PI4_ | 824::1461-1847::2118- |
| | kinase::FATC | 2368::2438-2470 |
| 400 | eIF-5a | 86-155 |
| 401 | KOW::eIF-5a | 26-60::84-151 |
| 402 | DS | 45-377 |
| 403 | Ribosomal_L18p | 26-173 |
| 404 | Orn Arg_deC_N::Orn_ | 91-326::329-460 |
| | DAP_Arg_deC | |
| 405 | IBN_N | 29-93 |
| 406 | SAM_decarbox | 23-396 |
| 407 | SAM_decarbox | 12-319 |
| 408 | SAM_decarbox | 12-346 |
| 409 | RB_A::RB_B | 274-475::594-721 |
| 410 | Gemini_AL1::Gemini_ | 9-127::129-233 |
| | AL1_M | |
| 411 | Globin::FAD_binding_ | 6-133::151-263::276-373 |
| | 6::NAD_binding_1 | |
| 412 | AP2 | 4-68 |
| 413 | FAE1_CUT1_RppA::ACP_ | 79-367::381-465 |
| | syn_III_C | |
| 414 | Cyclin_N::Cyclin_C | 189-315::317-441 |
| 415 | ABC_tran::ABC2_membrane:: | 186-386::503-715::724-887:: |
| | PDR_CDR::ABC_tran:: | 898-1087:1186-1404 |
| | ABC2_membrane | |
| 416 | Cyclin_N | 66-173 |
| 417 | Pkinase | 19-299 |
| 418 | Pkinase | 20-346 |
| 419 | PTR2 | 99-507 |
| 420 | PTR2 | 113-517 |
| 421 | RRM_1::RRM_1 | 98-165::216-286 |
| 422 | SET | 110-239 |
| 423 | HSF_DNA-bind | 173-416 |
| 424 | Clp_N::Clp_N::AAA::AAA_2 | 17-69::98-148::204-398::598-763 |
| 425 | Clp_N::Clp_N::AAA::AAA_2 | 17-69::94-145::201-395::596-760 |
| 426 | Clp_N::Clp_N::AAA::AAA_2 | 20-71::96-147::203-397::602-767 |
| 427 | Clp_N::Clp_N::AAA::AAA_2 | 17-69::94-145::201-395::596-763 |
| 428 | Cyclin_N | 47-183 |
| 429 | polyprenyl_synt | 37-308 |
| 430 | polyprenyl_synt | 45-316 |
| 431 | polyprenyl_synt | 47-318 |
| 432 | Cyclin_N | 56-202 |
| 433 | Cyclin_N::Cyclin_C | 79-193::195-327 |
| 434 | MtN3_slv::MtN3_slv | 6-95::128-214 |
| 435 | MtN3_slv::MIN3_slv | 7-96::129-215 |
| 436 | MtN3_slv::MIN3_slv | 8-77::125-211 |
| 437 | PAS::Pkinase | 111-222::480-732 |
| 438 | SET | 86-232 |
| 439 | Response_reg | 13-149 |
| 440 | Response_reg::Myb_ | 15-128::203-253 |
| | DNA-binding | |
| 441 | Response_reg::CCT | 26-142::660-698 |
| 442 | Response_reg::CCT | 44-160::588-626 |
| 443 | Response_reg::Myb_ | 26-139::213-263 |
| | DNA-binding | |
| 444 | Response_reg::Myb_ | 13-126::197-247 |
| | DNA-binding | |
| 445 | Response_reg | 10-139 |
| 446 | Response_reg | 12-135 |
| 447 | Response_reg | 42-177 |
| 448 | Response_reg | 37-157 |
| 449 | Response_reg::CCT | 28-153::457-495 |
| 450 | bZIP_1 | 64-128 |
| 451 | GRAS | 149-455 |
| 452 | GRAS | 162-497 |
| 453 | WD40::WD40::WD40:: | 56-94::98-136::147-186::194- |
| | WD40::WD40::WD40 | 234::239-277::334-372 |
| 454 | 14-3-3 | 7-242 |
| 455 | 14-3-3 | 7-242 |
| 456 | 14-3-3 | 9-246 |
| 457 | zf-NF-X1::zf-NF-X1:: | 209-227::262-281::315- |
| | zf-NF-X1::zf- | 334::369-389::423-442 |
| | NF-X1::zf-NF-X1 | |
| 458 | TAP42 | 30-367 |
| 459 | 14-3-3 | 5-241 |
| 460 | FBPase | 71-406 |
| 461 | FBPase | 2-329 |
| 462 | FBPase_glpX | 2-334 |
| 463 | FBPase | 18-341 |
| 464 | AAA | 217-404 |
| 465 | S1::S1::S1 | 603-676::1173-1245::1261-1336 |
| 466 | DUF902::DUF906 | 407-464::533-800 |
| 469 | CS | 5-79 |
| 470 | FKBP_C::FKBP_C::FKBP_ | 53-147::169-264::286- |
| | C::TPR_1::T-PR_1 | 383::452-485::486-519 |
| 471 | TPR_1::TPR_1:: | 5-38::40-73::74-107::262-295:: |
| | TPR_1::TPR_1::TPR_1:: | 336-369::396-429::430- |
| | TPR_1::TPR_1::TPR_1 | 463::464-497 |
| 472 | TPR_1::TPR_1 | 83-116::121-154 |
| 473 | Ribonuclease_T2 | 28-217 |
| 474 | GDA1_CD39 | 91-547 |
| 475 | Acid_phosphat_A | 65-399 |
| 476 | Sugar_tr | 22-517 |
| 477 | Sugar_tr | 26-520 |
| 478 | Citrate_synt | 47-413 |
| 479 | Citrate_synt | 46-409 |
| 480 | Citrate_synt | 78-455 |
| 481 | Citrate_synt | 90-458 |
| 482 | Citrate_synt | 100-468 |
| 483 | Ferritin | 88-233 |
| 484 | Ferritin | 91-236 |
| 485 | Ferritin | 7-144 |
| 486 | LEA_4::LEA_4 | 10-79::90-163 |
| 487 | HSF_DNA-bind | 15-189 |
| 488 | HSF_DNA-bind | 22-224 |
| 489 | DS | 44-361 |
| 490 | Carb_anhydrase | 75-310 |
| 491 | Carb_anhydrase | 38-264 |
| 492 | Mito_carr::Mito_ | 24-123::129-236::247-338 |
| | carr::Mito_carr | |
| 493 | Wzy_C | 311-377 |
| 494 | RNase_PH | 15-135 |
| 495 | DEAD::Helicase_C::DSHCT | 331-484::686-767::1094-1286 |
| 496 | TPR_1::TPR_1::TPR_ | 508-541::702-735::736- |
| | 1::TPR_1 | 769::1226-1259 |
| 498 | RNase_PH::RNase_PH_C | 21-153::156-220 |
| 499 | GTP_EFTU | 265-516 |
| 500 | GTP_EFTU::GTP_ | 391-619::641-708::713-821 |
| | EFTU_D2::GTP_EFTU_D3 | |
| 501 | TP_methylase | 4-211 |
| 502 | TP_methylase | 221-432 |
| 503 | TP_methylase | 120-333 |
| 504 | Asp | 85-441 |
| 505 | Asp | 148-505 |
| 506 | Asp | 139-476 |
| 509 | Dehydrin | 14-167 |
| 510 | Dehydrin | 25-286 |
| 515 | HSP9_HSP12 | 1-59 |
| 519 | F-box::LRR_2 | 17-64::299-323 |
| 520 | LRR_2::LRR_1:: | 389-414::415-437::465- |
| | LRR_1::LRR_1 | 489::568-591 |
| 521 | F-box::FBA_1 | 3-47::202-359 |
| 522 | F-box::LRR_2 | 62-108::414-438 |
| 523 | 2OG-Fell_Oxy | 158-258 |
| 524 | Aminotran_1_2 | 50-438 |
| 525 | FA_desaturase | 73-313 |
| 526 | Pyridoxal_deC | 63-412 |
| 527 | p450 | 40-480 |
| 528 | p450 | 44-477 |
| 529 | p450 | 60-515 |
| 530 | p450 | 42-496 |
| 531 | p450 | 73-511 |
| 532 | p450 | 41-466 |
| 533 | LRRNT_2::LRR_ | 127-167::194-216::218-240::266- |
| | 1::LRR_1::LRR_ | 288::290-312::314- |
| | 1::LRR_1::LRR_ | 336::338-360::362-384::458- |
| | 1::LRR_1::LRR_ | 480::551-573::575-597::598- |
| | 1::LRR_1::LRR_1::LRR_ | 620::646-668::670-692::694- |
| | 1::LRR_1::LRR_1::LRR_ | 716::718-741::754-776::778- |
| | 1::LRR_1::LRR_1::LRR_ | 800::826-848::851- |
| | 1::LRR_1::LRR_1::LRR_ | 870::875-894::927- |
| | 1::LRR_1::LRR_ | 949::951-973::1114-1396 |
| | 1::LRR_1::Pkinase | |
| 534 | E2F_TDP::E2F_TDP | 12-77::148-224 |
| 536 | E2F_TDP | 111-176 |
| 537 | Dicty_CAR | 14-321 |
| 538 | Mlo | 6-494 |
| 539 | Mlo | 32-520 |
| 540 | G-alpha | 12-376 |
| 542 | AP2 | 128-193 |
| 543 | Aa_trans | 32-427 |
| 544 | Aa_trans | 34-465 |
| 545 | AT_hook::AT_ | 151-163::214-226::294-306::324- |
| | hook::AT_hook::AT_ | 336::397-548::575- |
| | hook::YDG_SRA::Pre- | 675::677-830 |
| | SET::SET | |
| 546 | GRAS | 146-452 |
| 547 | MAT1 | 14-193 |
| 548 | Cystatin | 48-135 |
| 549 | Cystatin::Cystatin | 49-137::156-247 |
| 550 | Cystatin | 14-104 |
| 552 | PI3_PI4_kinase | 172-437 |
| 553 | DS | 47-363 |
| 554 | GRAS | 217-521 |
| 555 | GRAS | 165-471 |
| 556 | UQ_con | 20-159 |
| 557 | UPF0016::UPF0016 | 9-84::145-220 |
| 558 | AAA | 212-399 |
| 559 | CS | 5-81 |
| 560 | CS | 19-95 |
| 561 | CS | 5-81 |
| 562 | CS | 5-80 |
| 563 | Metallophos | 44-255 |
| 564 | Metallophos | 50-259 |
| 565 | Ribonuclease_T2 | 23-245 |
| 566 | Ribonuclease_T2 | 39-247 |
| 567 | Ribonuclease_T2 | 30-215 |
| 568 | Ribonuclease_T2 | 28-217 |
| 569 | HLH | 19-68 |
| 571 | RNase_PH::RNase_PH_C | 29-169::199-265 |
| 572 | 14-3-3 | 3-240 |
| 573 | 14-3-3 | 8-245 |
| 574 | IF4E | 5-206 |
| 575 | IF4E | 6-227 |
| 576 | IF4E | 7-210 |
| 577 | IF4E | 1-220 |
| 579 | GRAS | 154-464 |
| 580 | Catalase | 18-401 |
| 581 | Catalase | 18-402 |
| 582 | peroxidase | 17-224 |
| 583 | GDI | 1-438 |
| 584 | GDI | 1-452 |
| 585 | Rho_GDI | 35-245 |
| 586 | Cu_bind_like | 47-125 |
| 587 | Cu_bind_like | 42-120 |
| 588 | Cu_bind_like | 42-120 |
| 589 | Cu_bind_like | 45-105 |
| 590 | Cu_bind_like | 39-121 |
| 591 | ADH_zinc_N | 160-307 |
| 592 | ADH_zinc_N | 152-299 |
| 593 | ADH_zinc_N | 165-314 |
| 594 | ADH_N::ADH_zinc_N | 33-115::146-290 |
| 595 | Abhydrolase_1 | 175-412 |
| 596 | Hexapep::Hexapep:: | 65-82::91-108:117-134::135-152 |
| | Hexapep::Hexapep | |
| 597 | AhpC-TSA | 7-185 |
| 598 | AhpC-TSA | 5-182 |
| 599 | AhpC-TSA | 51-233 |
| 600 | Redoxin | 4-176 |
| 601 | AhpC-TSA | 69-248 |
| 602 | Redoxin | 68-211 |
| 603 | HSP20 | 134-240 |
| 604 | HSP20 | 77-181 |
| 605 | HSP20 | 85-182 |
| 606 | HSP20 | 60-163 |
| 607 | HSP20 | 50-153 |
| 609 | OPT | 104-758 |
| 610 | Xan_ur_permease | 35-432 |
| 611 | Xan_ur_permease | 38-445 |
| 612 | F-box::Tub | 57-112::123-480 |
| 613 | Tub | 1-251 |
| 614 | HMG_CoA_synt_N:: | 5-178::179-453 |
| | HMG_CoA_synt_C | |
| 615 | HMG_CoA_synt_N:: | 45-216::217-490 |
| | HMG_CoA_synt_C | |
| 616 | GRAS | 176-480 |
| 617 | Pkinase | 23-304 |
| 618 | E1-E2_ATPase::Hydrolase | 34-255::259-545 |
| 619 | E1-E2_ATPase | 225-473 |
| 621 | Hydrolase | 512-930 |
| 622 | Hydrolase | 457-898 |
| 623 | FBPase | 66-379 |
| 624 | FBPase | 13-337 |
| 625 | FBPase | 68-380 |
| 626 | FBPase | 63-374 |
| 627 | Myb_DNA-binding::Myb_ | 4-53::59-104 |
| | DNA-binding | |
| 628 | Myb_DNA-binding::Myb_ | 4-53::59-104 |
| | DNA-binding | |
| 629 | KNOX1::KNOX2:: | 88-132::135-186::232- |
| | ELK::Homeobox | 253::255-314 |
| 630 | KNOX1::KNOX2:: | 65-109::117-168::205- |
| | ELK::Homeobox | 226::228-287 |
| 631 | KNOX1::KNOX2:: | 57-101::104-155::202- |
| | ELK::Homeobox | 223::225-284 |
| 632 | bZ1P_1 | 227-289 |
| 633 | Myb_DNA-binding | 59-104 |
| 634 | Aa_trans | 27-433 |
| 635 | Aa_trans | 31-433 |
| 636 | Aa_trans | 59-459 |
| 637 | Sugar_tr | 26-487 |
| 638 | Sugar_tr | 26-489 |
| 639 | Sugar_tr | 29-489 |
| 640 | Sugar_tr | 29-552 |
| 641 | Sugar_tr | 101-535 |
| 642 | Sugar_tr | 53-503 |
| 643 | Sugar_tr | 47-479 |
| 644 | MFS_1 | 40-463 |
| 646 | Sugar_tr | 27-490 |
| 647 | Sugar_tr | 26-488 |
| 648 | p450 | 35-499 |
| 649 | WD40::WD40 | 160-197::249-288 |
| 650 | WD40::WD40 | 740-779::826-863 |
| 651 | HLH | 14-63 |
| 652 | HD-Z1P_N:: | 1-96::123-177::178-222 |
| | Homeobox::HALZ | |
| 654 | GH3 | 15-570 |
| 655 | Oxidored_FMN | 10-345 |
| 656 | Oxidored_FMN | 1-330 |
| 657 | Oxidored_FMN | 11-342 |
| 659 | TPR_1::TPR_2 | 78-111::112-145 |
| 661 | TPR_2::TPR_1::TPR_ | 2-35::36-69::70-103::253- |
| | 1::TPR_2::TPR_1::TPR_ | 286::287-320::328-365::392- |
| | 1::TPR_1::TPR_1:: | 425::426-459::460-493 |
| | TPR_1 | |
| 662 | TPR_1::TPR_1::TPR_2 | 124-157::158-191::192-225 |
| 663 | TPR_1::TPR_1::TPR_ | 14-47::48-81::82-115::195-269 |
| | 2::U-box | |
| 664 | TPR_1::TPR_1::TPR_ | 16-49::50-83::84-117::197-271 |
| | 1::U-box | |
| 665 | SRF-TF | 9-59 |
| 666 | SRF-TF::K-box | 9-59::69-173 |
| 667 | SRF-TF::K-box | 9-59::75-174 |
| 670 | CRAL_TRIO_ | 20-87::110-296 |
| | N::CRAL_TRIO | |
| 671 | CRAL_TRIO_ | 1-71:90-275 |
| | N::CRAL_TRIO | |
| 672 | CRAL_TRIO | 87-251 |
| 673 | CRAL_TRIO | 91-264 |
| 674 | CRAL_TRIO_ | 19-86::101-255 |
| | N::CRAL_TRIO | |
| 675 | Melhyltransf_7 | 36-369 |
| 676 | Methyltransf_7 | 36-382 |
| 677 | Methyltransf_7 | 38-378 |
| 678 | FtsH_ext:AAA:: | 77-223::249-436::4-43-653 |
| | Peplidase_M41 | |

TABLE 22

| PEP | | | | | |
|---|---|---|---|---|---|
| SEQ | Pfam domain | | | | |
| ID NO | name | begin | stop | score | E-value |
| 340 | Cellulose_synt | 167 | 977 | 2072.7 | 0 |
| 341 | AP2 | 67 | 129 | 130.5 | 4.20E−36 |
| 341 | B3 | 192 | 300 | 134 | 3.80E−37 |
| 342 | AP2 | 66 | 128 | 113 | 8.10E−31 |
| 342 | B3 | 181 | 294 | 124.3 | 3.30E−34 |
| 343 | AP2 | 64 | 126 | 104.4 | 3.00E−28 |
| 343 | B3 | 177 | 286 | 116.1 | 9.30E−32 |
| 344 | AP2 | 5 | 69 | 130.5 | 4.30E−36 |
| 345 | AP2 | 13 | 77 | 131 | 3.10E−36 |
| 346 | AP2 | 111 | 174 | 102.2 | 1.40E−27 |
| 346 | AP2 | 203 | 267 | 87.7 | 3.30E−23 |
| 347 | MIP | 11 | 231 | 379.7 | 4.00E−111 |
| 348 | Cyclin_N | 63 | 195 | 120.1 | 5.80E−33 |
| 348 | Cyclin_C | 197 | 317 | 19.9 | 0.00099 |
| 349 | Glyco_hydro_32N | 118 | 438 | 651.3 | 7.20E−193 |
| 349 | Glyco_hydro_32C | 479 | 601 | 147.9 | 2.40E−41 |
| 350 | Dicty_CAR | 12 | 328 | −10.2 | 5.20E−06 |
| 351 | KNOX1 | 102 | 146 | 90.4 | 5.10E−24 |
| 351 | KNOX2 | 153 | 204 | 101.2 | 2.90E−27 |
| 351 | ELK | 242 | 263 | 37 | 6.00E−08 |
| 351 | Homeobox | 273 | 324 | −1.9 | 0.0072 |
| 352 | CDC48_N | 30 | 116 | 134.7 | 2.30E−37 |
| 352 | AAA | 247 | 431 | 328 | 1.50E−95 |
| 352 | AAA_5 | 247 | 379 | 8.9 | 0.00035 |
| 352 | AAA | 520 | 707 | 344.1 | 2.10E−100 |
| 353 | AOX | 55 | 330 | 700.5 | 1.10E−207 |
| 354 | AOX | 26 | 333 | 421.3 | 1.30E−123 |
| 355 | Aa_trans | 32 | 471 | 375.7 | 6.60E−110 |
| 356 | PI3_PI4_kinase | 169 | 432 | 249.7 | 5.80E−72 |
| 359 | FA_desaturase | 156 | 400 | 352.8 | 5.40E−103 |
| 360 | FA_desaturase | 147 | 391 | 347.8 | 1.70E−101 |
| 361 | FA_desaturase | 140 | 384 | 347.7 | 1.80E−101 |
| 362 | PAS_2 | 70 | 186 | 222 | 1.20E−63 |
| 362 | GAF | 219 | 404 | 108.4 | 1.90E−29 |
| 362 | Phytochrome | 415 | 595 | 409.1 | 5.90E−120 |
| 362 | PAS | 622 | 737 | 96.6 | 6.70E−26 |
| 362 | PAS | 752 | 877 | 107.4 | 4.00E−29 |
| 362 | HisKA | 897 | 956 | 26.9 | 6.50E−05 |
| 362 | HATPase_c | 1011 | 1123 | 64.4 | 3.40E−16 |
| 363 | PAS_2 | 70 | 186 | 231.6 | 1.60E−66 |
| 363 | GAF | 219 | 404 | 108.7 | 1.60E−29 |
| 363 | Phytochrome | 415 | 595 | 406.5 | 3.50E−119 |
| 363 | PAS | 622 | 737 | 90.4 | 5.10E−24 |
| 363 | PAS_4 | 628 | 742 | 18.4 | 0.0029 |
| 363 | PAS | 752 | 877 | 97.5 | 3.60E−26 |
| 363 | HisKA | 897 | 956 | 31.6 | 2.50E−06 |
| 363 | HATPase_c | 1011 | 1123 | 61.2 | 3.10E−15 |
| 364 | PAS_2 | 105 | 226 | 209.2 | 8.50E−60 |
| 364 | GAF | 259 | 442 | 111.8 | 1.90E−30 |
| 364 | Phytochrome | 453 | 632 | 405.1 | 9.30E−119 |
| 364 | PAS | 663 | 779 | 117.2 | 4.40E−32 |
| 364 | PAS_4 | 669 | 784 | 19 | 0.0025 |
| 364 | PAS | 794 | 916 | 106.7 | 6.40E−29 |
| 364 | HisKA | 936 | 1000 | 45.6 | 1.50E−10 |
| 364 | HATPase_c | 1048 | 1160 | 60.9 | 3.90E−15 |
| 365 | PAS_2 | 114 | 234 | 214.8 | 1.80E−61 |
| 365 | GAF | 267 | 449 | 114.3 | 3.20E−31 |
| 365 | Phytochrome | 460 | 639 | 417 | 2.50E−122 |
| 365 | PAS | 670 | 786 | 118.1 | 2.40E−32 |
| 365 | PAS_4 | 676 | 791 | 22.5 | 0.0011 |
| 365 | PAS | 801 | 923 | 87.6 | 3.60E−23 |
| 365 | HisKA | 943 | 1007 | 54.8 | 2.60E−13 |
| 365 | HATPase_c | 1055 | 1167 | 56.9 | 6.20E−14 |
| 366 | PAS_2 | 68 | 184 | 237.8 | 2.10E−68 |
| 366 | GAF | 217 | 400 | 119.9 | 6.80E−33 |
| 366 | Phytochrome | 411 | 591 | 408.6 | 8.00E−120 |
| 366 | PAS | 622 | 737 | 88.5 | 1.90E−23 |
| 366 | PAS_4 | 628 | 742 | 18.5 | 0.0028 |
| 366 | PAS | 752 | 877 | 71.9 | 1.90E−18 |
| 366 | HisKA | 898 | 961 | 37.4 | 4.70E−08 |
| 366 | HATPase_c | 1009 | 1121 | 52.1 | 1.70E−12 |
| 367 | PAS_2 | 67 | 183 | 229.3 | 7.60E−66 |
| 367 | GAF | 216 | 399 | 119.3 | 1.00E−32 |
| 367 | Phytochrome | 410 | 590 | 383.7 | 2.50E−112 |
| 367 | PAS | 620 | 735 | 82.8 | 9.70E−22 |
| 367 | PAS | 750 | 875 | 78.3 | 2.20E−20 |
| 367 | HisKA | 896 | 959 | 38.9 | 1.60E−08 |
| 367 | HATPase_c | 1007 | 1121 | 61.9 | 1.90E−15 |
| 368 | Linker_histone | 21 | 97 | 27.1 | 1.80E−05 |
| 368 | AT_hook | 98 | 110 | 11.4 | 0.22 |
| 368 | AT_hook | 129 | 141 | 7.4 | 1.1 |
| 368 | AT_hook | 154 | 166 | 8.8 | 0.65 |
| 368 | AT_hook | 192 | 204 | 13.6 | 0.096 |
| 370 | GFO_IDH_MocA | 11 | 129 | 167.6 | 2.90E−47 |
| 370 | NAD_binding_3 | 17 | 128 | 7.5 | 0.00084 |
| 370 | GFO_IDH_MocA_C | 130 | 236 | 44.9 | 2.50E−10 |
| 371 | Cyclin_N | 54 | 186 | 115.8 | 1.20E−31 |
| 371 | Cyclin_C | 188 | 314 | 23.7 | 0.00051 |
| 372 | PAS | 116 | 230 | 22.8 | 0.0011 |
| 372 | PAS_3 | 141 | 233 | 22.8 | 0.00057 |
| 372 | PAS | 390 | 504 | 10.5 | 0.038 |
| 372 | PAS_3 | 415 | 507 | 20.3 | 0.00099 |
| 372 | Pkinase | 582 | 870 | 291.4 | 1.60E−84 |
| 373 | Globin | 17 | 157 | 113.2 | 6.90E−31 |
| 374 | Cyclin_N | 165 | 291 | 230.4 | 3.80E−66 |
| 374 | Cyclin_C | 293 | 413 | 191.2 | 2.30E−54 |
| 375 | Cyclin_N | 4 | 144 | 52.4 | 1.40E−12 |
| 376 | Cyclin_N | 157 | 283 | 241.8 | 1.40E−69 |
| 376 | Cyclin_C | 285 | 405 | 178.3 | 1.80E−50 |
| 377 | Cyclin_N | 243 | 370 | 235 | 1.50E−67 |
| 377 | Cyclin_C | 372 | 499 | 182.3 | 1.10E−51 |
| 378 | Cyclin_N | 166 | 292 | 221.3 | 2.00E−63 |
| 378 | Cyclin_C | 294 | 415 | 160.2 | 5.00E−45 |
| 379 | SRF-TF | 9 | 59 | 103 | 8.00E−28 |
| 379 | K-box | 69 | 172 | 38.7 | 1.80E−08 |
| 380 | SRF-TF | 13 | 63 | 94.5 | 3.00E−25 |
| 380 | K-box | 73 | 178 | 30.7 | 1.10E−06 |
| 381 | SRF-TF | 9 | 59 | 99.2 | 1.10E−26 |
| 381 | K-box | 72 | 171 | 30.3 | 1.10E−06 |
| 382 | SRF-TF | 9 | 59 | 99.2 | 1.10E−26 |
| 382 | K-box | 73 | 171 | 38.5 | 2.20E−08 |
| 383 | Cyclin_N | 244 | 371 | 237.7 | 2.20E−68 |
| 383 | Cyclin_C | 373 | 500 | 188.6 | 1.40E−53 |
| 384 | Cyclin_N | 104 | 233 | 228.8 | 1.10E−65 |
| 384 | Cyclin_C | 235 | 363 | 142 | 1.50E−39 |
| 385 | Cyclin_N | 163 | 289 | 221.7 | 1.50E−63 |
| 385 | Cyclin_C | 291 | 411 | 165.9 | 9.20E−47 |
| 386 | Cyclin_N | 228 | 354 | 221.6 | 1.70E−63 |
| 386 | Cyclin_C | 356 | 477 | 173.9 | 3.70E−49 |
| 387 | Cyclin_N | 173 | 299 | 229.1 | 8.80E−66 |
| 387 | Cyclin_C | 301 | 421 | 173.6 | 4.50E−49 |
| 388 | Cyclin_N | 187 | 312 | 228.5 | 1.30E−65 |
| 388 | Cyclin_C | 314 | 441 | 164.7 | 2.10E−46 |
| 389 | Cyclin_N | 47 | 190 | 39.2 | 1.30E−08 |
| 390 | Cyclin_N | 43 | 176 | 131.2 | 2.60E−36 |
| 390 | Cyclin_C | 178 | 298 | 18.6 | 0.0013 |
| 391 | Cyclin_N | 55 | 184 | 74.6 | 2.90E−19 |
| 392 | NDK | 75 | 209 | 338.6 | 9.90E−99 |
| 393 | NDK | 89 | 223 | 317.2 | 2.70E−92 |
| 394 | NDK | 2 | 134 | 312.4 | 7.30E−91 |
| 395 | NDK | 2 | 135 | 357.4 | 2.10E−104 |
| 396 | SNF2_N | 560 | 842 | 279.9 | 4.50E−81 |
| 396 | Helicase_C | 891 | 970 | 88.9 | 1.40E−23 |
| 398 | NDK | 33 | 170 | 137.8 | 2.80E−38 |
| 399 | HEAT | 248 | 284 | 14.6 | 0.33 |
| 399 | HEAT | 746 | 782 | 18.8 | 0.019 |
| 399 | HEAT | 787 | 824 | 27.7 | 3.90E−05 |
| 399 | FAT | 1461 | 1847 | 532.7 | 3.70E−157 |
| 399 | PI3_PI4_kinase | 2118 | 2368 | 376.4 | 4.00E−110 |
| 399 | FATC | 2438 | 2470 | 72.4 | 1.30E−18 |
| 400 | eIF-5a | 86 | 155 | 133.7 | 4.80E−37 |
| 401 | KOW | 26 | 60 | 30.5 | 5.40E−06 |
| 401 | eIF-5a | 84 | 151 | 151.5 | 2.10E−42 |
| 402 | DS | 45 | 377 | 776.6 | 1.40E−230 |
| 403 | Ribosomal_L18p | 26 | 173 | 282.5 | 7.40E−82 |
| 404 | Orn_Arg_deC_N | 91 | 326 | 431.3 | 1.30E−126 |
| 404 | Orn_DAP_Arg_deC | 329 | 460 | 140.4 | 4.60E−39 |
| 405 | IBN_N | 29 | 93 | 27.9 | 3.30E−05 |
| 406 | SAM_decarbox | 23 | 396 | 657.2 | 1.20E−194 |
| 407 | SAM_deearbox | 12 | 319 | 557.6 | 1.10E−164 |
| 408 | SAM_decarbox | 12 | 346 | 668.3 | 5.40E−198 |
| 409 | RB_A | 274 | 475 | 423.5 | 2.80E−124 |
| 409 | RB_B | 594 | 721 | 245.3 | 1.20E−70 |
| 410 | Gemini_AL1 | 9 | 127 | 269.6 | 5.70E−78 |
| 410 | Gemini_AL1_M | 129 | 233 | 190.4 | 3.90E−54 |
| 411 | Globin | 6 | 133 | 69.8 | 8.00E−18 |
| 411 | FAD_binding_6 | 151 | 263 | 30.4 | 3.50E−07 |
| 411 | NAD_binding_1 | 276 | 373 | 19.6 | 2.50E−05 |
| 412 | AP2 | 4 | 68 | 133.3 | 6.30E−37 |
| 413 | FAE1_CUT1_RppA | 79 | 367 | 749.5 | 2.00E−222 |
| 413 | Chal_sti_synt_C | 324 | 467 | 8.3 | 0.00033 |
| 413 | ACP_syn_III_C | 381 | 465 | 21.3 | 8.20E−08 |
| 414 | Cyclin_N | 189 | 315 | 212.9 | 6.80E−61 |
| 414 | Cyclin_C | 317 | 441 | 138.9 | 1.30E−38 |
| 415 | ABC_tran | 186 | 386 | 140.7 | 3.60E−39 |
| 415 | ABC2_membrane | 503 | 715 | 206.4 | 6.00E−59 |
| 415 | PDR_CDR | 724 | 887 | 213.4 | 4.70E−61 |
| 415 | ABC_tran | 898 | 1087 | 78 | 2.70E−20 |
| 415 | ABC2_membrane | 1186 | 1404 | 179.2 | 9.60E−51 |
| 416 | Cyclin_N | 66 | 173 | −1.1 | 0.00017 |
| 417 | Pkinase | 19 | 299 | 324 | 2.40E−94 |
| 418 | Pkinase | 20 | 346 | 243.6 | 3.90E−70 |
| 419 | PTR2 | 99 | 507 | 587.7 | 9.90E−174 |
| 420 | PTR2 | 113 | 517 | 353.1 | 4.20E−103 |
| 421 | RRM_1 | 98 | 165 | 22.9 | 0.001 |
| 421 | RRM_1 | 216 | 286 | 33 | 9.90E−07 |
| 422 | SET | 110 | 239 | 181.9 | 1.40E−51 |
| 423 | HSF_DNA-bind | 173 | 416 | 227.7 | 2.30E−65 |
| 424 | Clp_N | 17 | 69 | 33 | 9.70E−07 |
| 424 | Clp_N | 98 | 148 | 54.7 | 2.80E−13 |
| 424 | AAA | 204 | 398 | 53.6 | 6.00E−13 |
| 424 | AAA_2 | 598 | 763 | 366.2 | 4.70E−107 |
| 424 | AAA_5 | 602 | 768 | 21.2 | 3.90E−05 |
| 425 | Clp_N | 17 | 69 | 63.3 | 7.10E−16 |
| 425 | Clp_N | 94 | 145 | 55.2 | 2.00E−13 |
| 425 | AAA | 201 | 395 | 47.8 | 3.30E−11 |
| 425 | AAA_2 | 596 | 760 | 383.5 | 2.90E−112 |
| 425 | AAA_5 | 600 | 765 | 32.9 | 1.00E−06 |
| 426 | Clp_N | 20 | 71 | 60.3 | 5.90E−15 |
| 426 | Clp_N | 96 | 147 | 45.3 | 1.90E−10 |
| 426 | AAA | 203 | 397 | 50.6 | 4.80E−12 |
| 426 | AAA_2 | 602 | 767 | 377.8 | 1.50E−110 |
| 426 | AAA_5 | 606 | 768 | 26.5 | 1.60E−05 |
| 427 | Clp_N | 17 | 69 | 57 | 5.80E−14 |
| 427 | Clp_N | 94 | 145 | 52 | 1.80E−12 |
| 427 | AAA | 201 | 395 | 54.3 | 3.70E−13 |
| 427 | AAA_2 | 596 | 763 | 373.5 | 3.10E−109 |
| 427 | AAA_5 | 600 | 748 | 31.4 | 2.90E−06 |
| 428 | Cyclin_N | 47 | 183 | 48.7 | 1.80E−11 |
| 429 | polyprenyl_synt | 37 | 308 | 318.9 | 8.30E−93 |
| 430 | polyprenyl_synt | 45 | 316 | 353.8 | 2.60E−103 |
| 431 | polyprenyl_synt | 47 | 318 | 365 | 1.10E−106 |
| 432 | Cyclin_N | 56 | 202 | 70.9 | 3.70E−18 |
| 433 | Cyclin_N | 79 | 193 | 57 | 5.60E−14 |
| 433 | Cyclin_C | 195 | 327 | −2.1 | 0.052 |
| 434 | MtN3_slv | 6 | 95 | 79.7 | 8.40E−21 |
| 434 | MtN3_slv | 128 | 214 | 120.6 | 4.00E−33 |
| 435 | MtN3_slv | 7 | 96 | 94.5 | 2.90E−25 |
| 435 | MtN3_slv | 129 | 215 | 127.4 | 3.70E−35 |
| 436 | MtN3_slv | 8 | 77 | 20.5 | 9.60E−05 |
| 436 | MtN3_slv | 125 | 211 | 108.7 | 1.50E−29 |
| 437 | PAS | 111 | 222 | 63.2 | 7.80E−16 |
| 437 | PAS_4 | 117 | 227 | 34 | 4.70E−07 |
| 437 | PAS_3 | 133 | 225 | 18.8 | 0.0014 |
| 437 | Pkinase | 480 | 732 | 264.7 | 1.70E−76 |
| 437 | Pkinasc_Tyr | 480 | 732 | 257.2 | 3.20E−74 |
| 438 | SET | 86 | 232 | 142.5 | 1.00E−39 |
| 439 | Response_reg | 13 | 149 | 77.9 | 2.90E−20 |
| 440 | Response_reg | 15 | 128 | 95.3 | 1.70E−25 |
| 440 | Myb_DNA-binding | 203 | 253 | 48.6 | 1.90E−11 |
| 441 | Response_reg | 26 | 142 | 86.1 | 9.80E−23 |
| 441 | CCT | 660 | 698 | 74.9 | 2.40E−19 |
| 442 | Response_reg | 44 | 160 | 101.5 | 2.40E−27 |
| 442 | CCT | 588 | 626 | 79.5 | 9.70E−21 |
| 443 | Response_reg | 26 | 139 | 106.4 | 7.70E−29 |
| 443 | Myb_DNA-binding | 213 | 263 | 51.1 | 3.50E−12 |
| 444 | Response_reg | 13 | 126 | 104.9 | 2.20E−28 |
| 444 | Myb_DNA-binding | 197 | 247 | 46.3 | 9.50E−11 |
| 445 | Response_reg | 10 | 139 | 77.2 | 4.80E−20 |
| 446 | Response_reg | 12 | 135 | 82 | 1.70E−21 |
| 447 | Response_reg | 42 | 177 | 69.4 | 1.10E−17 |
| 448 | Response_reg | 37 | 157 | 88.2 | 2.30E−23 |
| 449 | Response_reg | 28 | 153 | 25.4 | 3.50E−05 |
| 449 | CCT | 457 | 495 | 70.6 | 4.80E−18 |
| 450 | bZIP_1 | 64 | 128 | 36.2 | 1.10E−07 |
| 450 | bZIP_2 | 64 | 118 | 35.5 | 1.80E−07 |
| 451 | GRAS | 149 | 455 | 424.5 | 1.30E−124 |
| 452 | GRAS | 162 | 497 | 270.9 | 2.30E−78 |
| 453 | WD40 | 56 | 94 | 42 | 1.90E−09 |
| 453 | WD40 | 98 | 136 | 23.6 | 0.00065 |
| 453 | WD40 | 147 | 186 | 35.3 | 1.90E−07 |
| 453 | WD40 | 194 | 234 | 34 | 4.90E−07 |
| 453 | WD40 | 239 | 277 | 45.9 | 1.20E−10 |
| 453 | WD40 | 334 | 372 | 24.1 | 0.00046 |
| 454 | 14-3-3 | 7 | 242 | 490.2 | 2.30E−144 |
| 455 | 14-3-3 | 7 | 242 | 509.9 | 2.70E−150 |
| 456 | 14-3-3 | 9 | 246 | 514.9 | 8.30E−152 |
| 457 | zf-NF-X1 | 209 | 227 | 19.9 | 0.0087 |
| 457 | zf-NF-X1 | 262 | 281 | 27.5 | 4.30E−05 |
| 457 | zf-NF-X1 | 315 | 334 | 20.6 | 0.005 |
| 457 | zf-NF-X1 | 369 | 389 | 25.2 | 0.00022 |
| 457 | zf-NF-X1 | 423 | 442 | 23.4 | 0.00076 |
| 458 | TAP42 | 30 | 367 | 617.5 | 1.10E−182 |
| 459 | 14-3-3 | 5 | 241 | 509.3 | 4.10E−150 |
| 460 | FBPase | 71 | 406 | 486.1 | 3.90E−143 |
| 461 | FBPase | 2 | 329 | 748.8 | 3.30E−222 |
| 462 | FBPase_glpX | 2 | 334 | 864.1 | 6.50E−257 |
| 463 | FBPase | 18 | 341 | 448.6 | 7.30E−132 |
| 464 | AAA | 217 | 404 | 296.6 | 4.40E−86 |
| 465 | S1 | 603 | 676 | 56.9 | 6.20E−14 |
| 465 | S1 | 1173 | 1245 | 45.3 | 1.90E−10 |
| 465 | S1 | 1261 | 1336 | 74.5 | 3.00E−19 |
| 466 | DUF902 | 407 | 464 | 117.4 | 3.70E−32 |
| 466 | DUF906 | 533 | 800 | 650.4 | 1.40E−192 |
| 469 | CS | 5 | 79 | 62.3 | 1.50E−15 |
| 470 | FKBP_C | 53 | 147 | 201.7 | 1.60E−57 |
| 470 | FKBP_C | 169 | 264 | 87.7 | 3.30E−23 |
| 470 | FKBP_C | 286 | 383 | 119.2 | 1.10E−32 |
| 470 | TPR_1 | 452 | 485 | 21.5 | 0.0027 |
| 470 | TPR_1 | 486 | 519 | 29.8 | 9.10E−06 |
| 470 | TPR_2 | 486 | 519 | 23.8 | 0.00057 |
| 471 | TPR_2 | 5 | 38 | 28.2 | 2.70E−05 |
| 471 | TPR_1 | 5 | 38 | 33.1 | 8.80E−07 |
| 471 | TPR_1 | 40 | 73 | 14.1 | 0.1 |
| 471 | TPR_2 | 74 | 107 | 33.7 | 6.00E−07 |
| 471 | TPR_1 | 74 | 107 | 39.8 | 8.50E−09 |
| 471 | TPR_1 | 262 | 295 | 16.5 | 0.053 |
| 471 | TPR_1 | 336 | 369 | 27.8 | 3.60E−05 |
| 471 | TPR_1 | 396 | 429 | 12.1 | 0.18 |
| 471 | TPR_1 | 430 | 463 | 39.8 | 8.70E−09 |
| 471 | TPR_2 | 430 | 463 | 24.4 | 0.00037 |
| 471 | TPR_1 | 464 | 497 | 9.4 | 0.37 |
| 472 | TPR_1 | 83 | 116 | 10.1 | 0.31 |
| 472 | TPR_1 | 121 | 154 | 34.2 | 4.10E−07 |
| 472 | TPR_2 | 121 | 154 | 23.3 | 0.00081 |
| 473 | Ribonuclease_T2 | 28 | 217 | 341.9 | 1.00E−99 |
| 474 | GDAI_CD39 | 91 | 547 | 87.7 | 3.30E−23 |
| 475 | Acid_phosphat_A | 65 | 399 | 324.4 | 1.80E−94 |
| 476 | Sugar_tr | 22 | 517 | 87.8 | 3.20E−23 |
| 476 | MFS_1 | 27 | 464 | 78.2 | 2.40E−20 |
| 477 | Sugar_tr | 26 | 520 | 84.3 | 3.40E−22 |
| 477 | MFS_1 | 30 | 467 | 75.2 | 1.80E−19 |
| 478 | Citrate_synt | 47 | 413 | 675 | 5.40E−200 |
| 479 | Citrate_synt | 46 | 409 | 799.2 | 2.10E−237 |
| 480 | Citrate_synt | 78 | 455 | 704.7 | 6.00E−209 |
| 481 | Citrate_synt | 90 | 458 | 508.2 | 8.60E−150 |
| 482 | Citrate_synt | 100 | 468 | 512.6 | 4.00E−151 |
| 483 | Ferritin | 88 | 233 | 224.9 | 1.60E−64 |
| 484 | Ferritin | 91 | 236 | 230.8 | 2.70E−66 |
| 485 | Ferritin | 7 | 144 | 163.6 | 4.60E−46 |
| 486 | LEA_4 | 10 | 79 | 33.1 | 9.00E−07 |
| 486 | LEA_4 | 90 | 163 | 76.1 | 1.00E−19 |
| 487 | HSF_DNA-bind | 15 | 189 | 226.5 | 5.40E−65 |
| 488 | HSF_DNA-bind | 22 | 224 | 161.9 | 1.50E−45 |
| 489 | DS | 44 | 361 | 611.1 | 9.30E−181 |
| 490 | Carb_anhydrase | 75 | 310 | 108.7 | 1.50E−29 |
| 491 | Carb_anhydrase | 38 | 264 | 150.2 | 5.20E−42 |
| 492 | Mito_carr | 24 | 123 | 82.9 | 9.50E−22 |
| 492 | Mito_carr | 129 | 236 | 101.7 | 2.00E−27 |
| 492 | Mito_carr | 247 | 338 | 96.1 | 9.70E−26 |
| 493 | Wzy_C | 311 | 377 | 72.1 | 1.60E−18 |
| 494 | RNase_PH | 15 | 135 | 60.2 | 6.40E−15 |
| 495 | DEAD | 331 | 484 | 123.9 | 4.20E−34 |
| 495 | Helicase_C | 686 | 767 | 25.2 | 8.20E−05 |
| 495 | DSHCT | 1094 | 1286 | 378.3 | 1.10E−110 |
| 496 | TPR_1 | 508 | 541 | 12.2 | 0.17 |
| 496 | TPR_1 | 702 | 735 | 8.4 | 0.49 |
| 496 | TPR_1 | 736 | 769 | 34.4 | 3.60E−07 |
| 496 | TPR_2 | 736 | 769 | 29.5 | 1.10E−05 |
| 496 | TPR_1 | 1226 | 1259 | 7.9 | 0.56 |
| 498 | RNase_PH | 21 | 153 | 152.2 | 1.30E−42 |
| 498 | RNase_PH_C | 156 | 220 | 53.7 | 5.50E−13 |
| 499 | GTP_EFTU | 265 | 516 | 52.8 | 1.10E−12 |
| 500 | GTP_EFTU | 391 | 619 | 253.2 | 5.10E−73 |
| 500 | GTP_EFTU_D2 | 641 | 708 | 43.2 | 8.10E−10 |
| 500 | GTP_EFTU_D3 | 713 | 821 | 45.5 | 1.70E−10 |
| 501 | TP_methylase | 4 | 211 | 321.1 | 1.80E−93 |
| 502 | TP_methylase | 221 | 432 | 292.6 | 6.90E−85 |
| 503 | TP_methylase | 120 | 333 | 257.4 | 2.70E−74 |
| 504 | Asp | 85 | 441 | −78.8 | 5.40E−09 |
| 505 | Asp | 148 | 505 | −71.2 | 1.90E−09 |
| 506 | Asp | 139 | 476 | −126.6 | 3.60E−06 |
| 509 | Dehydrin | 14 | 167 | 241.4 | 1.70E−69 |
| 510 | Dehydrin | 25 | 286 | 88.7 | 1.70E−23 |
| 515 | HSP9_HSPI2 | 1 | 59 | 150.8 | 3.40E−42 |
| 519 | F-box | 17 | 64 | 16.7 | 0.079 |
| 519 | LRR_2 | 299 | 323 | 12.3 | 0.31 |
| 520 | LRR_2 | 389 | 414 | 6.4 | 2 |
| 520 | LRR_1 | 415 | 437 | 7.9 | 8.9 |
| 520 | LRR_1 | 465 | 489 | 8.1 | 8 |
| 520 | LRR_1 | 568 | 591 | 7.8 | 9.4 |
| 521 | F-box | 3 | 47 | 40.7 | 4.70E−09 |
| 521 | FBA_1 | 202 | 359 | −34.4 | 0.0019 |
| 522 | F-box | 62 | 108 | 40.1 | 7.00E−09 |
| 522 | LRR_2 | 414 | 438 | 9.9 | 0.66 |
| 523 | 2OG-Fell_Oxy | 158 | 258 | 150.3 | 4.70E−42 |
| 524 | Aminotran_1_2 | 50 | 438 | 510.1 | 2.30E−150 |
| 525 | FA_desaturase | 73 | 313 | 316.4 | 4.60E−92 |
| 526 | Pyridoxal_deC | 63 | 412 | 151.7 | 1.70E−42 |
| 527 | p450 | 40 | 480 | 110.7 | 4.10E−30 |
| 528 | p450 | 44 | 477 | 184.2 | 2.90E−52 |
| 529 | p450 | 60 | 515 | 80.9 | 3.70E−21 |
| 530 | p450 | 42 | 496 | 111.6 | 2.20E−30 |
| 531 | p450 | 73 | 511 | 131.3 | 2.50E−36 |
| 532 | p450 | 41 | 466 | 200.1 | 4.70E−57 |
| 533 | LRRNT_2 | 127 | 167 | 27.1 | 5.70E−05 |
| 533 | LRR_1 | 194 | 216 | 11.3 | 2 |
| 533 | LRR_1 | 218 | 240 | 17.2 | 0.055 |
| 533 | LRR_1 | 266 | 288 | 13.4 | 0.78 |
| 533 | LRR_1 | 290 | 312 | 17.2 | 0.055 |
| 533 | LRR_1 | 314 | 336 | 11.9 | 1.6 |
| 533 | LRR_1 | 338 | 360 | 16.4 | 0.098 |
| 533 | LRR_1 | 362 | 384 | 19.9 | 0.0087 |
| 533 | LRR_1 | 458 | 480 | 18.8 | 0.018 |
| 533 | LRR_1 | 551 | 573 | 14.4 | 0.39 |
| 533 | LRR_1 | 575 | 597 | 10.4 | 3 |
| 533 | LRR_1 | 598 | 620 | 12.4 | 1.3 |
| 533 | LRR_1 | 646 | 668 | 13.6 | 0.65 |
| 533 | LRR_1 | 670 | 692 | 13.8 | 0.6 |
| 533 | LRR_1 | 694 | 716 | 20.3 | 0.0065 |
| 533 | LRR_1 | 718 | 741 | 12.6 | 1.1 |
| 533 | LRR_1 | 754 | 776 | 9 | 5.5 |
| 533 | LRR_1 | 778 | 800 | 8.2 | 7.6 |
| 533 | LRR_1 | 826 | 848 | 14.1 | 0.46 |
| 533 | LRR_1 | 851 | 870 | 12.1 | 1.5 |
| 533 | LRR_1 | 875 | 894 | 12.6 | 1.1 |
| 533 | LRR_1 | 927 | 949 | 15.1 | 0.24 |
| 533 | LRR_1 | 951 | 973 | 13.7 | 0.61 |
| 533 | Pkinase_Tyr | 1114 | 1396 | 115.4 | 1.50E−31 |
| 533 | Pkinase | 1114 | 1396 | 136.4 | 7.20E−38 |
| 534 | E2F_TDP | 12 | 77 | 115.1 | 1.90E−31 |
| 534 | E2F_TDP | 148 | 224 | 119 | 1.20E−32 |
| 536 | E2F_TDP | 111 | 176 | 137.7 | 2.80E−38 |
| 537 | Dicty_CAR | 14 | 321 | −22.2 | 3.10E−05 |
| 538 | Mlo | 6 | 494 | 1012 | 1.90E−301 |
| 539 | Mlo | 32 | 520 | 1031.3 | 0 |
| 540 | G-alpha | 12 | 376 | 553.4 | 2.20E−163 |
| 542 | AP2 | 128 | 193 | 140 | 5.90E−39 |
| 543 | Aa_trans | 32 | 427 | 170.2 | 5.00E−48 |
| 544 | Aa_trans | 34 | 465 | 480.5 | 1.90E−141 |
| 545 | AT_hook | 151 | 163 | 11.6 | 0.21 |
| 545 | AT_hook | 214 | 226 | 9.7 | 0.45 |
| 545 | AT_hook | 294 | 306 | 10.8 | 0.29 |
| 545 | AT_hook | 324 | 336 | 11.9 | 0.19 |
| 545 | YDG_SRA | 397 | 548 | 198.3 | 1.70E−56 |
| 545 | Pre-SET | 575 | 675 | 146 | 9.00E−41 |
| 545 | SET | 677 | 830 | 196.5 | 6.00E−56 |
| 546 | GRAS | 146 | 452 | 451.5 | 9.80E−133 |
| 547 | MAT1 | 14 | 193 | 1.1 | 1.10E−07 |
| 548 | Cystatin | 48 | 135 | 100.3 | 5.50E−27 |
| 549 | Cystatin | 49 | 137 | 68 | 2.80E−17 |
| 549 | Cystatin | 156 | 247 | 18.9 | 0.0033 |
| 550 | Cystatin | 14 | 104 | 62.1 | 1.60E−15 |
| 552 | PI3_PI4_kinase | 172 | 437 | 231.7 | 1.50E−66 |
| 553 | DS | 47 | 363 | 592.4 | 4.00E−175 |
| 554 | GRAS | 217 | 521 | 491 | 1.30E−144 |
| 555 | GRAS | 165 | 471 | 427.7 | 1.50E−125 |
| 556 | UQ_con | 20 | 159 | 187.8 | 2.50E−53 |
| 557 | UPF0016 | 9 | 84 | 102.1 | 1.60E−27 |
| 557 | UPF0016 | 145 | 220 | 111.7 | 2.00E−30 |
| 558 | AAA | 212 | 399 | 308.6 | 1.10E−89 |
| 558 | AAA_5 | 212 | 347 | 8 | 0.0004 |
| 559 | CS | 5 | 81 | 59.8 | 8.00E−15 |
| 560 | CS | 19 | 95 | 38.2 | 2.60E−08 |
| 561 | CS | 5 | 81 | 67.3 | 4.60E−17 |
| 562 | CS | 5 | 80 | 63.8 | 5.00E−16 |
| 563 | Metallophos | 44 | 255 | 74.5 | 3.20E−19 |
| 564 | Metallophos | 50 | 259 | 81.1 | 3.20E−21 |
| 565 | Ribonuclease_T2 | 23 | 245 | 252.4 | 8.60E−73 |
| 566 | Ribonuclease_T2 | 39 | 247 | 210 | 5.20E−60 |
| 567 | Ribonucicase_T2 | 30 | 215 | 93.2 | 7.00E−25 |
| 568 | Ribonuclease_T2 | 28 | 217 | 341.9 | 1.00E−99 |
| 569 | HLH | 19 | 68 | 62.5 | 1.30E−15 |
| 571 | RNase_PH | 29 | 169 | 100.2 | 5.60E−27 |
| 571 | RNase_PH_C | 199 | 265 | 20.7 | 0.0049 |
| 572 | 14-3-3 | 3 | 240 | 509.7 | 3.00E−150 |
| 573 | 14-3-3 | 8 | 245 | 508.7 | 6.20E−150 |
| 574 | IF4E | 5 | 206 | 413.1 | 3.70E−121 |
| 575 | IF4E | 6 | 227 | 480.9 | 1.40E−141 |
| 576 | IF4E | 7 | 210 | 385 | 1.10E−112 |
| 577 | IF4E | 1 | 220 | 424.8 | 1.10E−124 |
| 579 | GRAS | 154 | 464 | 462.7 | 4.30E−136 |
| 580 | Catalase | 18 | 401 | 955.4 | 2.00E−284 |
| 581 | Catalase | 18 | 402 | 954.1 | 5.00E−284 |
| 582 | peroxidase | 17 | 224 | 241.8 | 1.40E−69 |
| 583 | GD1 | 1 | 438 | 1048.3 | 0 |
| 584 | GD1 | 1 | 452 | 1080.8 | 0 |
| 585 | Rho_GDI | 35 | 245 | 92.5 | 1.20E−24 |
| 586 | Copper-bind | 36 | 132 | 4.5 | 0.00038 |
| 586 | Cu_bind_like | 47 | 125 | 137.2 | 4.10E−38 |
| 587 | Cu_bind_like | 42 | 120 | 113.6 | 5.20E−31 |
| 588 | Cu_bind_like | 42 | 120 | 149.2 | 9.80E−42 |
| 589 | Cu_bind_like | 45 | 105 | 58.1 | 2.60E−14 |
| 590 | Cu_bind_like | 39 | 121 | 55.8 | 1.30E−13 |
| 591 | ADH_zinc_N | 160 | 307 | 113.7 | 4.80E−31 |
| 592 | ADH_zinc_N | 152 | 299 | 101.7 | 2.00E−27 |
| 593 | ADH_zinc_N | 165 | 314 | 109.8 | 7.10E−30 |
| 594 | ADH_N | 33 | 115 | 74.6 | 3.00E−19 |
| 594 | ADH_zinc_N | 146 | 290 | 124.1 | 3.70E−34 |
| 595 | Abhydrolase_1 | 175 | 412 | 61.4 | 2.60E−15 |
| 596 | Hexapep | 65 | 82 | 13.8 | 0.57 |
| 596 | Hexapep | 91 | 108 | 14.1 | 0.48 |
| 596 | Hexapep | 117 | 134 | 8.9 | 11 |
| 596 | Hexapep | 135 | 152 | 14 | 0.49 |
| 597 | Redoxin | 6 | 161 | 57.9 | 3.00E−14 |
| 597 | AhpC-TSA | 7 | 185 | 368.3 | 1.10E−107 |
| 598 | Redoxin | 4 | 160 | 43.7 | 5.90E−10 |
| 598 | AhpC-TSA | 5 | 182 | 347.8 | 1.60E−101 |
| 599 | Redoxin | 50 | 210 | 29.4 | 1.20E−05 |
| 599 | AhpC-TSA | 51 | 233 | 380.8 | 1.90E−111 |
| 600 | Redoxin | 4 | 176 | 172.4 | 1.10E−48 |
| 601 | Redoxin | 68 | 224 | 56.6 | 7.50E−14 |
| 601 | AhpC-TSA | 69 | 248 | 400.8 | 1.90E−117 |
| 602 | Redoxin | 68 | 211 | 97.3 | 4.40E−26 |
| 602 | AhpC-TSA | 70 | 211 | −5.3 | 5.70E−11 |
| 603 | HSP20 | 134 | 240 | 137.9 | 2.50E−38 |
| 604 | HSP20 | 77 | 181 | 153.2 | 6.40E−43 |
| 605 | HSP20 | 85 | 182 | 30.7 | 5.10E−07 |
| 606 | HSP20 | 60 | 163 | 175.8 | 9.70E−50 |
| 607 | HSP20 | 50 | 153 | 185 | 1.70E−52 |
| 609 | OPT | 104 | 758 | 686.8 | 1.50E−203 |
| 610 | Xan_ur_permease | 35 | 432 | 176.9 | 4.60E−50 |
| 611 | Xan_ur_permease | 38 | 445 | 188.8 | 1.20E−53 |
| 612 | F-box | 57 | 112 | 32.1 | 1.90E−06 |
| 612 | Tub | 123 | 480 | 632.1 | 4.50E−187 |
| 613 | Tub | 1 | 251 | 393.2 | 3.50E−115 |
| 614 | HMG_CoA_synt_N | 5 | 178 | 338.3 | 1.20E−98 |
| 614 | HMG_CoA_synt_C | 179 | 453 | 549.9 | 2.40E−162 |
| 615 | HMG_CoA_synt_N | 45 | 216 | 426 | 4.80E−125 |
| 615 | HMG_CoA_synt_C | 217 | 490 | 622.4 | 3.50E−184 |
| 616 | GRAS | 176 | 480 | 418.1 | 1.10E−122 |
| 617 | Pkinase | 23 | 304 | 338 | 1.50E−98 |
| 618 | E1-E2_ATPase | 34 | 255 | 306.8 | 3.50E−89 |
| 618 | Hydrolase | 259 | 545 | 68 | 2.80E−17 |
| 619 | E1-E2_ATPase | 225 | 473 | −52.8 | 1.50E−06 |
| 621 | Hydrolase | 512 | 930 | 19.1 | 0.0013 |
| 622 | Hydrolase | 457 | 898 | 26.9 | 6.40E−05 |
| 623 | FBPase | 66 | 379 | 554.7 | 8.80E−164 |
| 624 | FBPase | 13 | 337 | 691.4 | 6.10E−205 |
| 625 | FBPase | 68 | 380 | 555.9 | 3.70E−164 |
| 626 | FBPase | 63 | 374 | 513.6 | 2.10E−151 |
| 627 | Myb_DNA-binding | 4 | 53 | 39.7 | 9.50E−09 |
| 627 | Myb_DNA-binding | 59 | 104 | 39.3 | 1.30E−08 |
| 628 | Myb_DNA-binding | 4 | 53 | 45 | 2.30E−10 |
| 628 | Myb_DNA-binding | 59 | 104 | 39.6 | 1.00E−08 |
| 629 | KNOX1 | 88 | 132 | 90.3 | 5.40E−24 |
| 629 | KNOX2 | 135 | 186 | 102.8 | 9.20E−28 |
| 629 | ELK | 232 | 253 | 37 | 6.10E−08 |
| 629 | Homeobox | 255 | 314 | −0.2 | 0.0048 |
| 630 | KNOX1 | 65 | 109 | 97 | 5.30E−26 |
| 630 | KNOX2 | 117 | 168 | 118.4 | 1.90E−32 |
| 630 | ELK | 205 | 226 | 29.8 | 8.60E−06 |
| 630 | Homeobox | 228 | 287 | 5.7 | 0.0012 |
| 631 | KNOX1 | 57 | 101 | 81.6 | 2.30E−21 |
| 631 | KNOX2 | 104 | 155 | 94.7 | 2.60E−25 |
| 631 | ELK | 202 | 223 | 30 | 7.60E−06 |
| 631 | Homeobox | 225 | 284 | 1.8 | 0.003 |
| 632 | bZIP_2 | 225 | 279 | 26.5 | 8.50E−05 |
| 632 | bZIP_1 | 227 | 289 | 29.2 | 1.40E−05 |
| 633 | Myb_DNA-binding | 59 | 104 | 58.3 | 2.30E−14 |
| 634 | Aa_trans | 27 | 433 | 475.6 | 5.50E−140 |
| 635 | Aa_trans | 31 | 433 | 508.5 | 6.80E−150 |
| 636 | Aa_trans | 59 | 459 | 295.7 | 7.90E−86 |
| 637 | Sugar_tr | 26 | 487 | 565 | 6.80E−167 |
| 637 | MFS_1 | 30 | 448 | 79.4 | 1.00E−20 |
| 638 | MFS_1 | 21 | 450 | 89.5 | 9.40E−24 |
| 638 | Sugar_tr | 26 | 489 | 611.3 | 7.90E−181 |
| 639 | Sugar_tr | 29 | 489 | 392.1 | 7.60E−115 |
| 639 | MFS_1 | 33 | 449 | 75.6 | 1.40E−19 |
| 640 | Sugar_tr | 29 | 552 | 421.5 | 1.10E−123 |
| 640 | MFS_1 | 33 | 511 | 90.8 | 3.90E−24 |
| 641 | Sugar_tr | 101 | 535 | 347.7 | 1.80E−101 |
| 641 | MFS_1 | 105 | 494 | 80.9 | 3.60E−21 |
| 642 | Sugar_tr | 53 | 503 | 427.7 | 1.50E−125 |
| 642 | MFS_1 | 57 | 462 | 125.4 | 1.50E−34 |
| 643 | Sugar_tr | 47 | 479 | 287.4 | 2.60E−83 |
| 643 | MFS_1 | 52 | 439 | 77 | 5.60E−20 |
| 644 | Sugar_tr | 37 | 468 | -46.3 | 1.90E−05 |
| 644 | MFS_1 | 40 | 463 | 26.4 | 1.80E−05 |
| 646 | Sugar_tr | 27 | 490 | 468.6 | 7.10E−138 |
| 646 | MFS_1 | 33 | 447 | 86.5 | 7.80E−23 |
| 647 | Sugar_tr | 26 | 488 | 522.3 | 4.70E−154 |
| 647 | MFS_1 | 41 | 445 | 61.1 | 3.30E−15 |
| 648 | p450 | 35 | 499 | 310 | 4.00E−90 |
| 649 | WD40 | 160 | 197 | 27.3 | 5.10E−05 |
| 649 | WD40 | 249 | 288 | 33.1 | 8.90E−07 |
| 650 | WD40 | 740 | 779 | 35.7 | 1.50E−07 |
| 650 | WD40 | 826 | 863 | 30.7 | 4.80E−06 |
| 651 | HLH | 14 | 63 | 60.2 | 6.30E−15 |
| 652 | HD-ZIP_N | 1 | 96 | 151.2 | 2.60E−42 |
| 652 | Homeobox | 123 | 177 | 65.2 | 1.90E−16 |
| 652 | HALZ | 178 | 222 | 86.1 | 1.00E−22 |
| 654 | GH3 | 15 | 570 | 1262.5 | 0 |
| 655 | Oxidored_FMN | 10 | 345 | 295.2 | 1.10E−85 |
| 656 | Oxidored_FMN | 1 | 330 | 262.8 | 6.30E−76 |
| 657 | Oxidored_FMN | 11 | 342 | 332 | 9.60E−97 |
| 659 | TPR_1 | 78 | 111 | 22.5 | 0.0014 |
| 659 | TPR_1 | 112 | 145 | 22.3 | 0.0016 |
| 659 | TPR_2 | 112 | 145 | 22.5 | 0.0014 |
| 661 | TPR_2 | 2 | 35 | 30.9 | 4.20E−06 |
| 661 | TPR_1 | 2 | 35 | 29.1 | 1.40E−05 |
| 661 | TPR_1 | 36 | 69 | 9.3 | 0.39 |
| 661 | TPR_2 | 70 | 103 | 34 | 4.70E−07 |
| 661 | TPR_1 | 70 | 103 | 37.3 | 4.80E−08 |
| 661 | TPR_2 | 253 | 286 | 27.8 | 3.50E−05 |
| 661 | TPR_1 | 253 | 286 | 27.1 | 5.70E−05 |
| 661 | TPR_2 | 287 | 320 | 21 | 0.0038 |
| 661 | TPR_1 | 287 | 320 | 28.8 | 1.80E−05 |
| 661 | TPR_1 | 328 | 365 | 11.3 | 0.22 |
| 661 | TPR_2 | 392 | 425 | 27.2 | 5.30E−05 |
| 661 | TPR_1 | 392 | 425 | 33.7 | 5.90E−07 |
| 661 | TPR_2 | 426 | 459 | 23.4 | 0.00074 |
| 661 | TPR_1 | 426 | 459 | 34.2 | 4.20E−07 |
| 661 | TPR_2 | 460 | 493 | 24.8 | 0.00029 |
| 661 | TPR_1 | 460 | 493 | 35.6 | 1.60E−07 |
| 662 | TPR_1 | 124 | 157 | 14.2 | 0.099 |
| 662 | TPR_1 | 158 | 191 | 26.4 | 9.40E−05 |
| 662 | TPR_1 | 192 | 225 | 16.2 | 0.058 |
| 662 | TPR_2 | 192 | 225 | 21.3 | 0.0033 |
| 663 | TPR_1 | 14 | 47 | 22.2 | 0.0017 |
| 663 | TPR_2 | 14 | 47 | 20.6 | 0.0053 |
| 663 | TPR_2 | 48 | 81 | 23.3 | 0.00078 |
| 663 | TPR_1 | 48 | 81 | 33.1 | 8.90E−07 |
| 663 | TPR_1 | 82 | 115 | 12.8 | 0.15 |
| 663 | TPR_2 | 82 | 115 | 21.1 | 0.0036 |
| 663 | U-box | 195 | 269 | 132.5 | 1.10E−36 |
| 664 | TPR_1 | 16 | 49 | 23.2 | 0.00086 |
| 664 | TPR_2 | 16 | 49 | 20.7 | 0.005 |
| 664 | TPR_1 | 50 | 83 | 29.3 | 1.30E−05 |
| 664 | TPR_1 | 84 | 117 | 11.9 | 0.19 |
| 664 | U-box | 197 | 271 | 125.9 | 1.00E−34 |
| 665 | SRF-TF | 9 | 59 | 80.2 | 6.00E−21 |
| 666 | SRF-TF | 9 | 59 | 92.5 | 1.20E−24 |
| 666 | K-box | 69 | 173 | 31.2 | 9.50E−07 |
| 667 | SRF-TF | 9 | 59 | 120.8 | 3.60E−33 |
| 667 | K-box | 75 | 174 | 154.8 | 2.00E−43 |
| 670 | CRAL_TRIO_N | 20 | 87 | 119 | 1.30E−32 |
| 670 | CRAL_TRIO | 110 | 296 | 350.5 | 2.60E−102 |
| 671 | CRAL_TRIO_N | 1 | 71 | 30.9 | 4.20E−06 |
| 671 | CRAL_TRIO | 90 | 275 | 25.8 | 7.70E−08 |
| 672 | CRAL_TRIO | 87 | 251 | 65 | 2.20E−16 |
| 673 | CRAL_TRIO | 91 | 264 | 88.5 | 1.90E−23 |
| 674 | CRAL_TRIO_N | 19 | 86 | 28 | 2.30E−05 |
| 674 | CRAL_TRIO | 101 | 255 | 68.7 | 1.70E−17 |
| 675 | Methyltransf_7 | 36 | 369 | 629.7 | 2.40E−186 |
| 676 | Methyltransf_7 | 36 | 382 | 371.9 | 9.00E−109 |
| 677 | Methyltransf_7 | 38 | 378 | 384 | 2.10E−112 |
| 678 | FtsH_ext | 77 | 223 | 137 | 4.70E−38 |
| 678 | AAA | 249 | 436 | 336.6 | 3.90E−98 |
| 678 | AAA_5 | 249 | 384 | 5.9 | 0.00059 |
| 678 | Pcptidase_M41 | 443 | 653 | 399 | 6.30E−117 |

TABLE 23

| Pfam domain | Accession | Gathering | |
|---|---|---|---|
| name | number | cutoff | Domain description |
| 14-3-3 | PF00244.9 | 25 | 14-3-3 protein |
| 2OG-Fell_Oxy | PF03171.10 | 11.5 | 2OG-Fe(II) oxygenase |
| | | | superfamily |
| AAA | PF00004.19 | 12.3 | ATPase family associated |
| | | | with various cellular |
| | | | activities (AAA) |
| AAA_2 | PF07724.3 | −5 | ATPase family associated |
| | | | with various cellular |
| | | | activities (AAA) |
| AAA_5 | PF07728.4 | 4 | ATPase family_associated |
| | | | with various cellular |
| | | | activities (AAA) |
| ABC2_membrane | PF01061.13 | −17.9 | ABC-2 type transporter |
| ABC_tran | PF00005.16 | 9.5 | ABC transporter |
| ACP_syn_III_C | PF08541.1 | −24.4 | 3-Oxoacyl-[acyl-carrier- |
| | | | protein (ACP)) synthase |
| | | | III C terminal |
| ADH_N | PF08240.2 | −14.5 | Alcohol dehydrogenase |
| | | | GroES-like domain |
| ADH_zinc_N | PF00107.16 | 23.8 | Zinc-binding dehydrogenase |
| AOX | PF01786.8 | 25 | Alternative oxidase |
| AP2 | PF00847.10 | 0 | AP2 domain |
| AT_hook | PF02178.8 | 3.6 | AT hook motif |
| Aa_trans | PF01490.7 | −128.4 | Transmembrane amino |
| | | | acid transporter protein |
| Abhydrolase_1 | PF00561.10 | 10.3 | alpha/beta hydrolase fold |
| Acid_phosphat_A | PF00328.12 | −64.5 | Histidine acid phosphatase |
| AhpC-TSA | PF00578.10 | −92.2 | AhpC/TSA family |
| Aminotran_1_2 | PF00155.11 | −57.5 | Aminotransferase class |
| | | | I and II |
| Asp | PF00026.13 | −153.8 | Eukaryotic aspartyl protease |
| B3 | PF02362.12 | 26.5 | B3 DNA binding domain |
| CCT | PF06203.4 | 25 | CCT motif |
| CDC48_N | PF02359.8 | −2 | Cell division protein 48 |
| | | | (CDC48), N-terminal domain |
| CRAL_TRIO | PF00650.9 | −26 | CRAL/TRIO domain |
| CRAL_TRIO_N | PF03765.4 | 16 | CRAL/TRIO, N-terminus |
| CS | PF04969.6 | 8.6 | CS domain |
| Carb_anhydrase | PF00194.10 | −105 | Eukaryotic-type carbonic |
| | | | anhydrase |
| Catalase | PF00199.9 | −229 | Catalase |
| Cellulose_synt | PF03552.4 | −257.9 | Cellulose synthase |
| Chal_sti_synt_C | PF02797.5 | −6.1 | Chalcone and stilbene |
| | | | synthases, C-terminal domain |
| Citrate_synt | PF00285.11 | −101.5 | Citrate synthase |
| Clp_N | PF02861.10 | 0 | Clp amino terminal domain |
| Copper-bind | PF00127.10 | −7.7 | Copper binding proteins, |
| | | | plastocyanin/azurin family |
| Cu_bind_like | PF02298.7 | −16.4 | Plastocyanin-like domain |
| Cyclin_C | PF02984.9 | −13 | Cyclin, C-terminal domain |
| Cyclin_N | PF00134.13 | −14.7 | Cyclin, N-terminal domain |
| Cystatin | PF00031.11 | 17.5 | Cystatin domain |
| DEAD | PF00270.18 | 7.2 | DEAD/DEAN box helicase |
| DS | PF01916.7 | −95.2 | Deoxyhypusine synthase |
| DSHCT | PF08148.1 | −86.9 | DSHCT (NUC185) domain |
| DUF902 | PF06001.2 | 25 | Domain of Unknown |
| | | | Function (DUF902) |
| DUF906 | PF06010.1 | 25 | Domain of Unknown |
| | | | Function (DUF906) |
| Dehydrin | PF00257.10 | −4.4 | Dehydrin |
| Dicty_CAR | PF05462.2 | −39.7 | Slime mold cyclic AMP |
| | | | receptor |
| E1-E2_ATPase | PF00122.9 | −84 | E1-E2 ATPase |
| E2F_TDP | PF02319.11 | 17 | E2F/DP family winged-helix |
| | | | DNA-binding domain |
| ELK | PF03789.3 | 25 | ELK domain |
| F-box | PF00646.22 | 13.8 | F-box domain |
| FAD_binding_6 | PF00970.13 | −11.4 | Oxidoreductase FAD- |
| | | | binding domain |
| FAE1_CUT1_ | PF08392.2 | −192.7 | FAE1/Type III polyketide |
| RppA | | | synthase-like protein |
| FAT | PF02259.12 | 275 | FAT domain |
| FATC | PF02260.9 | 20 | FATC domain |
| FA_desaturase | PF00487.14 | −46 | Fatty acid desaturase |
| FBA_1 | PF07734.2 | −39.4 | F-box associated |
| FBPase | PF00316.10 | −170.3 | Fructose-1-6-bisphosphatase |
| FBPase_glpX | PF03320.4 | −198.1 | Bacterial fructose-1,6- |
| | | | bisphosphatase, |
| | | | glpX-encoded |
| FKBP_C | PF00254.17 | −7.6 | FKBP-type peptidyl- |
| | | | prolyl cis-trans isomerase |
| Ferritin | PF00210.14 | −10 | Ferritin-like domain |
| FtsH_ext | PF06480.4 | 25 | FtsH Extracellular |
| G-alpha | PF00503.9 | −230 | G-protein alpha subunit |
| GAF | PF01590.15 | 23 | GAF domain |
| GDA1_CD39 | PF01150.7 | −183 | GDA1/CD39 (nucleoside |
| | | | phosphatase) family |
| GDI | PF00996.8 | −285.8 | GDP dissociation inhibitor |
| GFO_IDH-1_ | PF01408.12 | −4.4 | Oxidoreductase family, |
| MocA | | | NAD-binding Rossmann fold |
| GFO_IDH_ | PF02894.7 | 6 | Oxidoreductase family, |
| MocA_C | | | C-terminal alpha/beta domain |
| GH3 | PF03321.3 | −336 | GH3 auxin-responsive |
| | | | promoter |
| GRAS | PF03514.5 | −78 | GRAS family |
| | | | transcription factor |
| GTP_EFTU | PF00009.16 | 8 | Elongation factor Tu |
| | | | GTP binding domain |
| GTP_EFTU_D2 | PF03144.15 | 25 | Elongation factor Tu |
| | | | domain 2 |
| GTP_EFTU_D3 | PF03143.6 | 14.3 | Elongation factor Tu |
| | | | C-terminal domain |
| Gemini_AL1 | PF00799.10 | −38.7 | Geminivirus Rep |
| | | | catalytic domain |
| Gemini_AL1_M | PF08283.1 | −3 | Geminivirus rep protein |
| | | | central domain |
| Globin | PF00042.11 | −8.8 | Globin |
| Glyco_hydro_32C | PF08244.2 | 8.8 | Glycosyl hydrolases |
| | | | family 32 C terminal |
| Glyco_hydro_32N | PF00251.10 | −197 | Glycosyl hydrolases |
| | | | family 32 N terminal |
| HALZ | PF02183.7 | 17 | Homeobox associated |
| | | | leucine zipper |
| HATPase_c | PF02518.15 | 22.4 | Histidine kinase-, DNA |
| | | | gyrase B-, and |
| | | | HSP90-like ATPase |
| HD-ZIP_N | PF04618.2 | 25 | HD-ZIP protein N terminus |
| HEAT | PF02985.11 | 11.5 | HEAT repeat |
| HLH | PF00010.15 | 8.2 | Helix-loop-helix |
| | | | DNA-binding domain |
| HMG_CoA_ | PF08540.1 | −158.1 | Hydroxymethylglutaryl- |
| synt_C | | | coenzyme A synthase |
| | | | C terminal |
| HMG_CoA_ | PF01154.8 | −6.2 | Hydroxymethylglutaryl- |
| synt_N | | | coenzyme A |
| | | | synthase N terminal |
| HSF_DNA-bind | PF00447.7 | −70 | HSF-type DNA-binding |
| HSP20 | PF00011.10 | 13 | Hsp20/alpha crystallin family |
| HSP9_HSP12 | PF04119.2 | 25 | Heat shock protein 9/12 |
| Helicase_C | PF00271.20 | 2.1 | Helicase conserved |
| | | | C-terminal domain |
| Hexapep | PF00132.13 | 0.3 | Bacterial transferase |
| | | | hexapeptide (three repeats) |
| HisKA | PF00512.14 | 10.3 | His Kinase A |
| | | | (phosphoacceptor) domain |
| Homeobox | PF00046.18 | −4.1 | Homeobox domain |
| Hydrolase | PF00702.15 | 13.6 | haloacid dehalogenase- |
| | | | like hydrolase |
| IBN_N | PF03810.9 | 21.9 | Importin-beta |
| | | | N-terminal domain |
| IF4E | PF01652.8 | −35 | Eukaryotic initiation |
| | | | factor 4E |
| K-box | PF01486.7 | 0 | K-box region |
| KNOX1 | PF03790.3 | 25 | KNOX1 domain |
| KNOX2 | PF03791.3 | 25 | KNOX2 domain |
| KOW | PF00467.18 | 29.1 | KOW motif |
| LEA_4 | PF02987.6 | 0 | Late embryogenesis |
| | | | abundant protein |
| LRRNT_2 | PF08263.3 | 18.6 | Leucine rich repeat |
| | | | N-terminal domain |
| LRR_1 | PF00560.22 | 7.7 | Leucine Rich Repeat |
| LRR_2 | PF07723.2 | 6 | Leucine Rich Repeat |
| Linker_histone | PF00538.8 | -8 | linker histone H1 and |
| | | | H5 family |
| MAT1 | PF06391.2 | −55.1 | CDK-activating kinase |
| | | | assembly factor MAT1 |
| MFS_1 | PF07690.6 | 23.5 | Major Facilitator Superfamily |
| MIP | PF00230.10 | −62 | Major intrinsic protein |
| Metallophos | PF00149.18 | 22 | Calcineurin-like |
| | | | phosphoesterase |
| Methyltransf_7 | PF03492.5 | 25 | SAM dependent carboxyl |
| | | | methyltransferase |
| Mito_carr | PF00153.16 | 0 | Mitochondrial carrier protein |
| Mlo | PF03094.5 | −263 | Mlo family |
| MIN3_slv | PF03083.5 | 9.7 | MtN3/saliva family |
| Myb_DNA- | PF00249.20 | 14 | Myb-like DNA-binding |
| binding | | | domain |
| NAD_binding_1 | PF00175.11 | −3.9 | Oxidoreductase NAD- |
| | | | binding domain |
| NAD_binding_3 | PF03447.6 | −1.7 | Homoserine dehydrogenase, |
| | | | NAD binding domain |
| NDK | PF00334.9 | −59.9 | Nucleoside diphosphate kinase |
| OPT | PF03169.6 | −238.6 | OPT oligopeptide |
| | | | transporter protein |
| Orn_Arg_deC_N | PF02784.7 | −76 | Pyridoxal-dependent |
| | | | decarboxylase, |
| | | | pyridoxal binding domain |
| Orn_DAP_ | PF00278.12 | 6.7 | Pyridoxal-dependent |
| Arg_deC | | | decarboxylase, |
| | | | C-terminal sheet domain |
| Oxidored_FMN | PF00724.9 | −147.7 | NADH:flavin oxidoreductase/ |
| | | | NADH oxidase family |
| PAS | PF00989.13 | 0 | PAS fold |
| PAS_2 | PF08446.1 | −2.1 | PAS fold |
| PAS_3 | PF08447.1 | 13.4 | PAS fold |
| PAS_4 | PF08448.1 | 16.4 | PAS fold |
| PDR_CDR | PF06422.2 | −51.8 | CDR ABC transporter |
| PI3_PI4_kinase | PF00454.16 | 14.8 | Phosphatidylinositol |
| | | | 3- and 4-kinase |
| PTR2 | PF00854.12 | −50 | POT family |
| Peptidase_M41 | PF01434.8 | −139.8 | Peptidase family M41 |
| Phytochrome | PF00360.9 | 13 | Phytochrome region |
| Pkinase | PF00069.15 | −70.3 | Protein kinase domain |
| Pkinase_Tyr | PF07714.6 | 65 | Protein tyrosine kinase |
| Pre-SET | PF05033.5 | 3.9 | Pre-SET motif |
| Pyridoxal_deC | PF00282.9 | −158.6 | Pyridoxal-dependent |
| | | | decarboxylase |
| | | | conserved domain |
| RB_A | PF01858.7 | −65.3 | Retinoblastoma-associated |
| | | | protein A domain |
| RB_B | PF01857.9 | −48.7 | Retinoblastoma-associated |
| | | | protein B domain |
| RNase_PH | PF01138.10 | 4 | 3′exoribonuclease |
| | | | family, domain 1 |
| RNase_PH_C | PF03725.4 | 20 | 3′exoribonuclease |
| | | | family, domain 2 |
| RRM_1 | PF00076.12 | 17.7 | RNA recognition motif. |
| | | | (a.k.a. RRM, RBD, |
| | | | or RNP domain) |
| Redoxin | PF08534.1 | −1 | Redoxin |
| Response_reg | PF00072.13 | 4 | Response regulator |
| | | | receiver domain |
| Rho_GDI | PF02115.6 | −55 | RHO protein GDP |
| | | | dissociation inhibitor |
| Ribonuclease_T2 | PF00445.8 | −53 | Ribonuclease T2 family |
| Ribosomal_L18p | PF00861.12 | 25 | Ribosomal L18p/L5e family |
| S1 | PF00575.13 | 16.8 | S1 RNA binding domain |
| SAM_decarbox | PF01536.6 | −154 | Adenosylmethionine |
| | | | decarboxylase |
| SET | PF00856.17 | 23.5 | SET domain |
| SNF2_N | PF00176.13 | −72 | SNF2 family N-terminal |
| | | | domain |
| SRF-TF | PF00319.8 | 11 | SRF-type transcription factor |
| | | | (DNA-binding and |
| | | | dimerisation domain) |
| Sugar_tr | PF00083.14 | −85 | Sugar (and other) transporter |
| TAP42 | PF04177.3 | 25 | TAP42-like family |
| TPR_1 | PF00515.17 | 7.7 | Tetratricopeptide repeat |
| TPR_2 | PF07719.6 | 20.1 | Tetratricopeptide repeat |
| TP_methylase | PF00590.10 | −38 | Tetrapyrrote (Corrin/ |
| | | | Porphyrin) Methylases |
| Tub | PF01167.7 | −98 | Tub family |
| U-box | PF04564.6 | −7.6 | U-box domain |
| UPF0016 | PF01169.8 | 25 | Uncharacterized protein |
| | | | family UPF0016 |
| UQ_con | PF00179.16 | −30 | Ubiquitin-conjugating enzyme |
| WD40 | PF00400.21 | 21.5 | WD domain, G-beta repeat |
| Wzy_C | PF04932.4 | 25 | O-Antigen Polymerase |
| Xan_ur_permease | PF00860.11 | −151.2 | Permease family |
| YDG_SRA | PF02182.7 | 25 | YDG/SRA domain |
| bZIP_1 | PF00170.11 | 24.5 | bZIP transcription factor |
| bZIP_2 | PF07716.5 | 15 | Basic region leucine zipper |
| eIF-5a | PF01287.9 | 9.6 | Eukaryotic initiation factor |
| | | | 5A hypusine, DNA-binding |
| | | | OB fold |
| p450 | PF00067.11 | −105 | Cytochrome P450 |
| peroxidase | PF00141.12 | −10 | Peroxidase |
| polyprenyl_synt | PF00348.8 | −43 | Polyprenyl synthetase |
| zf-NF-X1 | PF01422.7 | 3 | NF-X1 type zinc finger |

### Example 9

### Selection of Transgenic Plants with Enhanced Agronomic Trait(s)

This example illustrates the preparation and identification by selection of transgenic seeds and plants derived from transgenic plant cells of this invention where the plants and seed are identified by screening for a transgenic plant having an enhanced agronomic trait imparted by expression of a protein selected from the group including the homologous proteins identified in Example 6. Transgenic plant cells of corn, soybean, cotton, canola, alfalfa, wheat and rice are transformed with recombinant DNA for expressing each of the homologs identified in Example 6. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. Plants are identified exhibiting enhanced traits imparted by expression of the homologous proteins.

1-12. (canceled)

13. A transgenic plant cell comprising a stably integrated, recombinant DNA transgene, wherein the recombinant DNA transgene comprises a promoter that is functional in said plant cell and that is operably linked to a protein coding DNA sequence encoding a protein comprising an amino acid sequence with at least 80% identity to SEQ ID NO: 411.

14. The transgenic plant cell of claim 13, wherein the amino acid sequence of the protein has at least 90% identity to SEQ ID NO: 411.

15. The transgenic plant cell of claim 13, further comprising a selectable marker transgene that provides tolerance from exposure to an herbicide.

16. The transgenic plant cell of claim 15, wherein said herbicide is glyphosate, dicamba, or glufosinate.

17. A transgenic plant comprising the transgenic plant cell of claim 13.

18. The transgenic plant of claim 17, which is homozygous for said recombinant DNA transgene.

19. The transgenic plant of claim 17, wherein the transgenic plant has an enhanced agronomic trait as compared to a wild type plant, and wherein the enhanced agronomic trait is increased yield, enhanced disease resistance, enhanced water stress resistance, and/or reduced shade avoidance.

20. A transgenic seed comprising the transgenic plant cell of claim 13.

21. The transgenic seed of claim 19, wherein the transgenic seed is from a corn, soybean, cotton, canola, alfalfa, wheat or rice plant.

22. A transgenic pollen grain comprising a haploid gamete from the transgenic plant of claim 17, wherein the gamete comprises the recombinant DNA transgene.

23. A method for producing a transgenic plant comprising:
(a) transforming plant cells with a recombinant DNA transgene, wherein the recombinant DNA transgene comprises a promoter that is functional in said plant cell and that is operably linked to a protein coding DNA sequence encoding a flavohemoglobin protein comprising an amino acid sequence with at least 80% identity to SEQ ID NO: 411;
(b) regenerating plants from said transformed plant cells; and
(c) selecting one or more transgenic plants transformed with the recombinant DNA transgene.

25. The method of claim 23, wherein the plant cells are further transformed with a selectable marker transgene, and wherein the one or more transgenic plants are selected based on the presence of said selectable marker transgene providing tolerance to a selection agent.

26. The method of claim 23, wherein the one or more transgenic plants are selected by detecting the presence of the transgene.

27. The method of claim 23, further comprising:
selecting one or more transgenic plants having an enhanced agronomic trait, wherein the enhanced agronomic trait comprises increased yield, enhanced disease resistance, enhanced water stress resistance, and/or reduced shade avoidance relative to a corresponding untransformed plant.

28. A recombinant DNA construct comprising a protein coding DNA sequence encoding a flavohemoglobin protein comprising an amino acid sequence with at least 70% identity to SEQ ID NO: 411 operably linked to a promoter functional in plant cells.

29. The recombinant DNA construct of claim 28, wherein the amino acid sequence of the flavohemoglobin protein is at least 90% identical to SEQ ID NO: 411.
